(19) United States
(12) Patent Application Publication
(10) Pub. No.: US 2024/0209454 A1
(43) Pub. Date: Jun. 27, 2024

(54) USE OF MUTATIONAL SIGNATURES FOR MULTIPLE CANCER TYPES

(71) Applicants: Deirdre Hill, Albuquerque, NM (US); Eric R. Prossnitz, Albuquerque, NM (US); Yan Guo, Albuquerque, NM (US)

(72) Inventors: Deirdre Hill, Albuquerque, NM (US); Eric R. Prossnitz, Albuquerque, NM (US); Yan Guo, Albuquerque, NM (US)

(21) Appl. No.: 18/556,049

(22) PCT Filed: Apr. 19, 2022
(86) PCT No.: PCT/US2022/025344

§ 371 Date: Oct. 18, 2023

Related U.S. Application Data
(60) Provisional application No. 63/176,562, filed on Apr. 19, 2021.

(51) Int. Cl. C12Q 1/6886; G16B 20/20

(52) U.S. Cl. CPC C12Q 1/6886; G16B 20/20; C12Q 2600/106; C12Q 2600/156

(57) ABSTRACT

A method to determine disease-specific survival, disease-free interval, progression-free interval, progression-free survival or overall survival in a cancer patient based on specific mutational signatures is provided.

### CROSS-REFERENCE TO RELATED APPLICATIONS

This application claims the benefit of the filing date of U.S. application No. 63/176,562, filed on Apr. 19, 2021, the disclosure of which is incorporated by reference herein.

### BACKGROUND

Somatic mutations that arise in solid tumors are currently understood to be predominantly a consequence of genomic instability, evoked by a handful of established driver mutations. In contrast with this random mutagenesis that accompanies tumor progression, investigators have also identified a small proportion that constitute regular patterns of mutation occurrence. Such distinct, reoccurring changes in the tumor genome have been recognized as signatures or “fingerprints” of exposure to specific environmental mutagens, endogenous processes, or defective DNA repair (Alexandrov et al., 2020; Alexandrov et al., 2013; Alexandrov et al., 2014). Mutational signatures have been identified and validated via experimental carcinogenesis in cancer cell and stem cell lines, yeast, and other model systems (Koh et al., 2020). Oncogenic mechanisms as well as exposures implicated in etiology have been highlighted by the specific mutational processes identified.

While mechanistic and etiologic understanding has been enhanced by mutational signature identification, signatures have seldom been evaluated in relation to clinical outcomes. Individuals with DNA repair defects, in particular, have had altered outcomes following chemotherapy in several studies (Gryfe et al., 2000; Ribic et al., 2003). In one recent investigation, women with triple-negative breast tumors, for instance, were more likely to harbor homologous repair defects in tumors, and responded more favorably to chemotherapy (Staaf et al., 2019). Such findings suggest that signatures of DNA repair deficiency and other mutational processes may have unrealized clinical utility.

### SUMMARY

As disclosed herein single base substitution signatures, covering a range of environmental agent and endogenous exposures, as well as defective DNA repair pathways, were identified that were associated with cancer survival. Algorithms for mutational signature assessment were applied to elucidate cancer-specific outcomes.

In particular, sixteen cancers met inclusion criteria for in-depth analysis, although information on 33 total cancers is included. Of the 49 signatures, 36 were associated with DSS in at least one cancer. Most common signatures influencing survival included a clock-like signature associated with age in six cancers, deregulated APOBEC-related cytosine deamination in five, and defective DNA repair also in five. Patients with signatures of tobacco exposure had increased risks of cancer-specific mortality in breast (4-fold), low grade glioma (2.2 to 2.8-fold), and skin cutaneous melanoma (3-fold). Some signatures related to DSS were also implicated in stage III/IV disease (Table 2). Addition of mutational signatures increased the c-index in all cancers. After signature inclusion, tumor mutational burden (TMB) did not add further significant prognostic discrimination. Mutational signatures may influence clinical outcomes. Signatures not previously linked to DSS may shed light on metastasis-related mechanisms, and may allow for improved understanding of poor prognosis tumors.

Other combinations of signatures may be employed to in methods for determining disease-free interval, progression-free interval survival, progression-free survival or overall survival in a patient.

The methods may be employed to select patients who should begin therapy sooner, may benefit from a specific therapy or may delay therapy, e.g., chemotherapy, radiotherapy or other immune therapies such as checkpoint inhibitor therapy.

In one embodiment, the patient has Adrenocortical carcinoma (ACC), Bladder Urothelial Carcinoma (BLCA), Bladder Urothelial Carcinoma (BLCA), Brain Lower Grade Glioma (LGG), Breast invasive carcinoma (BRCA), Cervical squamous cell carcinoma and endocervical adenocarcinoma (CESC), Cholangiocarcinoma (CHOL), Colon adenocarcinoma (COAD), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Liver hepatocellular carcinoma (LIHC), Lung adenocarcinoma (LUAD), Lung squamous cell carcinoma (LUSC), Lymphoid Neoplasm Diffuse Large B-cell b Lymphoma (DLBC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thymoma (THYM), Thyroid carcinoma (THCA), Uterine Carcinosarcoma (UCS), or Uterine Corpus Endometrial Carcinoma (UCEC).

The use of the identified signatures may be employed to direct therapy, e.g., the signatures may identify an individual that will benefit from one therapy over another, identify an individual that is responding well to a therapy or identify an individual where therapy should be discontinued, e.g., replaced with another therapy, in specific cancers as not every signature is associated with all cancers examined. Moreover, the use of the signatures may identify an individual who should be aggressively treated versus an individual where watchful waiting or hospice is indicated.

### BRIEF DESCRIPTION OF THE FIGURES

FIGS. 1a-1g. Relationship between disease-specific survival (DSS) and endogenous signature as represented by: a) Clock-like age (SBS1); b) APOBEC (SBS2); c) Mismatch repair deficiency (SBS6); d) Polymerase epsilon defects (SBS10b); e) APOBEC (SBS13); f) Mismatch repair deficiency (SBS26); g) base excision repair deficiency (SBS30). Endogenous processes with more than one SBS signature are labeled with that signature number.

FIGS. 2a-2d. Survival plots for a) BLCA and SBS2, b) BRCA and SBS15, c) HNSC and SBS85, d) LGG and SBS30] Legend FIG. 2: Time-to-event for endogenous processes related to disease-specific survival (DSS): a) APOBEC in BLCA, b) Mismatch repair in BRCA; c) Activation-induced cytosine deaminase in HNSC; d) Base excision repair in LGG.

FIGS. 3a-3g. Relationship between disease-specific survival (DSS) and exogenous mutagen signature as represented by: a) Ultraviolet (SBS7a); b) Ultraviolet (SBS7b); c) Ultraviolet (SBS7c); d) Aristolochic acid (SBS22); e) Aflatoxin (SBS24); f) Chemotherapy (SBS25); g) Platinum chemotherapy (SB31). Exogenous mutagens with more than one signature are labeled with that signature number.

FIGS. 4a-4d. Survival plots for a) BLCA and SBS22, b) COAD and SBS18, c) LGG and SBS4, d) SKCM and SBS7a] Legend FIG. 4: Time-to-event for exogenous mutagens related to disease-specific survival (DSS): a) Aristolochic acid in BLCA, b) Reactive oxygen species in COAD; c) Tobacco (SBS4) in LGG; d) Ultraviolet in SKCM.

### DETAILED DESCRIPTION

A method to determine disease-specific, disease-free interval, progression-free, progression-free interval, and/or overall survival in a cancer patient is provided. In one embodiment, the method includes obtaining a tumor sample from a patient with, for example, breast cancer (BRCA), bladder cancer (BLCA), colon adenocarcinoma (COAD), brain lower grade glioma (LGG), liver hepatocellular carcinoma (LIHC), ovarian serous cystadenocarcinoma (OV), stomach adenocarcinoma (STAD), uterine corpus endometrial carcinoma (UCEC), skin cutaneous melanoma (SKC), cervical squamous cell carcinoma and endocervical adenocarcinoma (CESC), head and neck squamous cell carcinoma (HNSC), lung squamous cell carcinoma (LUSC) or pancreatic adenocarcinoma (PAAD); and determining if the tumor sample has one or more mutational signatures comprising one or more of the mutational signatures in Table 2, or any combination thereof, wherein the presence of the one or more signatures in Table 2 in the sample relative to a corresponding sample without the one or more signatures in Table 2 is indicative of disease-specific survival in the patient. In one embodiment, the one or more signatures are selected from SBS1, SBS2, SBS6, SBS10a, SBS10b, SBS13, SBS15, SBS20, SBS26, or SBS30, or any combination thereof. In one embodiment, the cancer is COAD, LGG, LIHC, OV, STAD, or UCEC. In one embodiment, the cancer is BRCA, COAD, LIHC, or STAD. In one embodiment, the cancer is BLCA, BRCA, LGG, SKC or STAD. In one embodiment, the cancer is LIHC or HNSC. In one embodiment, the sample is from a stage I cancer. In one embodiment, the sample is from a stage III/IV cancer. In one embodiment, one of the mutational signatures in Table 2 is detected. In one embodiment, two or more of the mutational signatures in Table 2 are detected. In one embodiment, up to thirteen of the mutational signatures in Table 2 are detected. In one embodiment, the presence of SBS1 is detected. In one embodiment, the presence of SBS2 or SBS13, or both, is detected. In one embodiment, the presence of SBS6, 15, 20, or 26, or any combination, is detected. In one embodiment, the presence of SBS30 is detected. In one embodiment, the presence of SBS10a or SBS10b, or both, is detected. In one embodiment, the presence of the one or more mutational signatures is indicative of increased survival. In one embodiment, the presence of the one or more mutational signatures is indicative of decreased survival. In one embodiment, the one or more mutational signatures are detected using a nucleic acid amplification reaction. In one embodiment, the one or more mutational signatures are detected using a probe. In one embodiment, the one or more mutational signatures are detected using sequencing. In one embodiment, the presence of the one or more mutational signatures is indicative of response to therapy. In one embodiment, the presence of the one or more mutational signatures is indicative of a need for therapy, e.g., due to increased risk of disease-specific mortality. In one embodiment, the therapy is radiotherapy. In one embodiment, the therapy is chemotherapy. In one embodiment, the therapy is immunotherapy. In one embodiment, the therapy is antibody therapy.

An algorithm that detects mutational signatures correlated with disease-specific survival is disclosed in Blokzijl et al. (2018), which is incorporated by reference herein. The method includes detecting in a tumor sample from a cancer patient whole exome, whole genome, or targeted sequence(s), the presence of one or more mutational signatures comprising one or more of the mutational signatures in Table 2, or any combination thereof, wherein the presence of the one or more signatures in Table 2 in the sample relative to a corresponding sample without the one or more signatures in Table 2 is indicative of altered disease-specific or disease-free survival in the patient.

The method includes detecting in a tumor sample whole exome, whole genome, or targeted sequence from a cancer patient the presence of one or more mutational signatures comprising one or more of the mutational signatures in Table 2, wherein the presence of the one or more signatures in Table 2 in the sample relative to a corresponding sample without the one or more signatures in Table 2 is indicative of altered overall survival in the patient.

The method includes detecting in a tumor sample, e.g., via whole exome, whole genome, targeted or other methods of detecting a sequence in a tumor sample, from a cancer patient the presence of one or more mutational signatures comprising one or more of the mutational signatures in Table 2, wherein the presence of the one or more signatures in Table 2 in the sample relative to a corresponding sample without the one or more signatures in Table 2 is indicative of altered progression-free survival in the patient

The method includes detecting in a tumor sample whole exome, whole genome, or targeted sequence from a cancer patient the presence of one or more mutational signatures comprising one or more of the mutational signatures in Table 2, wherein the presence of the one or more signatures in Table 2 in the sample relative to a corresponding sample without the one or more signatures in Table 2 is indicative of altered progression-free interval survival in the patient.

In one embodiment, the one or more signatures are selected from SBS1, SBS2, SBS6, SBS10a, SBS10b, SBS13, SBS15, SBS20, SBS26, or SBS30, or any combination thereof. In one embodiment, the cancer is COAD, LGG, LIHC, OV, STAD, or UCEC. In one embodiment, the cancer is BRCA, COAD, LIHC, or STAD. In one embodiment, the cancer is BLCA, BRCA, LGG, SKC or STAD. In one embodiment, the cancer is LIHC or HNSC. In one embodiment, the sample is from a stage I or stage II cancer. In one embodiment, the sample is from a stage III/IV cancer. In one embodiment, one of the mutational signatures in Table 2 is detected. In one embodiment, two or more of the mutational signatures in Table 2 are detected. In one embodiment, up to thirteen of the mutational signatures in Table 2 are detected. In one embodiment, the presence of SBS1 is detected. In one embodiment, the presence of SBS2 or SBS13, or both, is detected. In one embodiment, the presence of SBS6, 15, 20, or 26, or any combination, is detected. In one embodiment, the presence of SBS30 is detected. In one embodiment, the presence of SBS10a or SBS10b, or both, is detected. In one embodiment, the presence of the one or more mutational signatures is indicative of increased survival. In one embodiment, the presence of the one or more mutational signatures is indicative of decreased survival. In one embodiment, the one or more mutational signatures are detected using a nucleic acid amplification reaction. In one embodiment, the one or more mutational signatures are detected using a probe. In one embodiment, the one or more mutational signatures are detected using sequencing. In one embodiment, the presence of the one or more mutational signatures is indicative of response to therapy. In one embodiment, the presence of the one or more mutational signatures is indicative of a need for therapy, e.g., due to increased risk of disease-specific mortality. In one embodiment, the therapy is radiotherapy. In one embodiment, the therapy is chemotherapy. In one embodiment, the therapy is immunotherapy. In one embodiment, the therapy is antibody therapy. In one embodiment, the therapy is targeted therapy.

A method to determine disease-specific survival in a cancer patient is provided. The method includes obtaining a tumor sample from a patient with any cancer and determining if the tumor sample has one or more mutational signatures comprising one or more of the mutational signatures in Table 2, or any combination thereof, wherein the presence of the one or more signatures in Table 2 in the sample relative to a corresponding sample without the one or more signatures in Table 2 is indicative of altered disease-specific, progression-free, disease-free, or overall survival in the patient. In one embodiment, the one or more signatures are selected from SBS1, SBS2, SBS6, SBS10a, SBS10b, SBS13, SBS15, SBS20, SBS26, or SBS30, or any combination thereof. In one embodiment, the cancer is COAD, LGG, LIHC, OV, STAD, or UCEC. In one embodiment, the cancer is BRCA, COAD, LIHC, or STAD. In one embodiment, the cancer is BLCA, BRCA, LGG, SKC or STAD. In one embodiment, the cancer is LIHC or HNSC. In one embodiment, the sample is from a stage I cancer. In one embodiment, the sample is from a stage III/IV cancer. In one embodiment, one of the mutational signatures in Table 2 is detected. In one embodiment, two or more of the mutational signatures in Table 2 are detected. In one embodiment, up to thirteen of the mutational signatures in Table 2 are detected. In one embodiment, the presence of SBS1 is detected. In one embodiment, the presence of SBS2 or SBS13, or both, is detected. In one embodiment, the presence of SBS6, 15, 20, or 26, or any combination, is detected. In one embodiment, the presence of SBS30 is detected. In one embodiment, the presence of SBS10a or SBS10b, or both, is detected. In one embodiment, the presence of the one or more mutational signatures is indicative of increased survival. In one embodiment, the presence of the one or more mutational signatures is indicative of decreased survival. In one embodiment, the one or more mutational signatures are detected using a nucleic acid amplification reaction. In one embodiment, the one or more mutational signatures are detected using a probe. In one embodiment, the one or more mutational signatures are detected using sequencing. In one embodiment, the presence of the one or more mutational signatures is indicative of response to therapy. In one embodiment, the presence of the one or more mutational signatures is indicative of a need for therapy, e.g., due to increased risk of disease-specific mortality. In one embodiment, the therapy is radiotherapy. In one embodiment, the therapy is chemotherapy. In one embodiment, the therapy is immunotherapy. In one embodiment, the therapy is antibody therapy. The term “anticancer agent” or “additional anticancer agent” (depending on the context of its use) shall mean chemotherapeutic agents such as an agent selected from the group consisting of microtubule-stabilizing agents, microtubule-disruptor agents, alkylating agents, antimetabolites, epidophyllotoxins, antineoplastic enzymes, topoisomerase inhibitors, inhibitors of cell cycle progression, and platinum coordination complexes. These may be selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]-benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258,); 3-[5-(methylsulfonylpiperadinemethyl)-indolyl]-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)x where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, among others. In one embodiment, the therapy includes administration of a checkpoint inhibitor. In one embodiment, the inhibitor comprises pembrolizumab, nivolumab, atezolizumab, avelumab, durvalumab, or ipilimumab.

### EXEMPLARY EMBODIMENTS

A method to detect mutational signatures correlated with disease-specific survival is provided. The method includes detecting in a tumor sample from a cancer patient the presence of one or more mutational signatures comprising one or more of the mutational signatures in Table 2, wherein the presence of the one or more signatures in Table 2 in the sample relative to a corresponding sample without the one or more signatures in Table 2 is indicative of disease-specific survival in the patient. In one embodiment, the one or more signatures are selected from SBS1, SBS2, SBS6, SBS10a, SBS10b, SBS13, SBS15, SBS20, SBS26, or SBS30, or any combination thereof. In one embodiment, the cancer is COAD, LGG, LIHC, OV, STAD, or UCEC. In one embodiment, the cancer is BRCA, COAD, LIHC, or STAD. In one embodiment, the cancer is BLCA, BRCA, LGG, SKC or STAD. In one embodiment, the cancer is LIHC or HNSC. In one embodiment, the sample is from a stage I cancer. In one embodiment, the sample is from a stage II/III/IV cancer. In one embodiment, one of the mutational signatures in Table 2 is detected. In one embodiment, two or more of the mutational signatures in Table 2 are detected. In one embodiment, up to thirteen of the mutational signatures in Table 2 are detected. In one embodiment, the presence of SBS1 is detected. In one embodiment, the presence of SBS2 or SBS13, or both, is detected. In one embodiment, the presence of SBS6, 15, 20, or 26, or any combination, is detected. In one embodiment, the presence of SBS30 is detected. In one embodiment, the presence of SBS10a or SBS10b, or both, is detected. In one embodiment, the presence of the one or more mutational signatures is indicative of increased survival. In one embodiment, the presence of the one or more mutational signatures is indicative of decreased survival. In one embodiment, the one or more mutational signatures are detected using a nucleic acid amplification reaction. In one embodiment, the one or more mutational signatures are detected using a probe. In one embodiment, the one or more mutational signatures are detected using sequencing. In one embodiment, the sequencing is specific for the one or more mutational signatures. In one embodiment, the presence of the one or more mutational signatures is indicative of response to therapy. In one embodiment, the presence of the one or more mutational signatures is indicative of a need for therapy. In one embodiment, the therapy is radiotherapy. In one embodiment, the therapy is chemotherapy. In one embodiment, the therapy is immunotherapy. In one embodiment, the therapy is antibody therapy.

Also provided is a method to determine disease-specific survival in a cancer patient, including obtaining a tumor sample from a patient with breast cancer (BRCA), bladder cancer (BLCA), colon adenocarcinoma (COAD), brain lower grade glioma (LGG), liver hepatocellular carcinoma (LIHC), ovarian serous cystadenocarcinoma (OV), stomach adenocarcinoma (STAD), uterine corpus endometrial carcinoma (UCEC), skin cutaneous melanoma (SKC), cervical squamous cell carcinoma and endocervical adenocarcinoma (CESC), head and neck squamous cell carcinoma (HNSC), lung squamous cell carcinoma (LUSC) or pancreatic adenocarcinoma (PAAD); and determining if the tumor sample has one or more mutational signatures comprising one or more of the mutational signatures in Table 2, wherein the presence of the one or more signatures in Table 2 in the sample relative to a corresponding sample without the one or more signatures in Table 2 is indicative of disease-specific survival in the patient. In one embodiment, the one or more signatures are selected from SBS1, SBS2, SBS6, SBS10a, SBS10b, SBS13, SBS15, SBS20, SBS26, or SBS30, or any combination thereof. In one embodiment, the cancer is COAD, LGG, LIHC, OV, STAD, or UCEC. In one embodiment, the cancer is BRCA, COAD, LIHC, or STAD. In one embodiment, the cancer is BLCA, BRCA, LGG, SKC or STAD. In one embodiment, the cancer is LIHC or HNSC. In one embodiment, the sample is from a stage I cancer. In one embodiment, the sample is from a stage II/III/IV cancer. In one embodiment, one of the mutational signatures in Table 2 is detected. In one embodiment, two or more of the mutational signatures in Table 2 are detected. In one embodiment, up to thirteen of the mutational signatures in Table 2 are detected. In one embodiment, the presence of SBS1 is detected. In one embodiment, the presence of SBS2 or SBS13, or both, is detected. In one embodiment, the presence of SBS6, 15, 20, or 26, or any combination, is detected. In one embodiment, the presence of SBS30 is detected. In one embodiment, the presence of SBS10a or SBS10b, or both, is detected. In one embodiment, the presence of the one or more mutational signatures is indicative of increased survival. In one embodiment, the presence of the one or more mutational signatures is indicative of decreased survival. In one embodiment, the one or more mutational signatures are detected using a nucleic acid amplification reaction. In one embodiment, the one or more mutational signatures are detected using a probe. In one embodiment, the one or more mutational signatures are detected using sequencing. In one embodiment, the sequencing is specific for the one or more mutational signatures. In one embodiment, the presence of the one or more mutational signatures is indicative of response to therapy. In one embodiment, the presence of the one or more mutational signatures is indicative of a need for therapy. In one embodiment, the therapy is radiotherapy. In one embodiment, the therapy is chemotherapy. In one embodiment, the therapy is immunotherapy. In one embodiment, the therapy is antibody therapy.

Also provided is a method to detect mutational signatures correlated with disease-specific survival, disease-free interval, progression-free interval, progression-free survival or overall survival, comprising:
detecting in a tumor sample from a cancer patient the presence of one or more mutational signatures comprising one or more of the mutational signatures in Table 2, or any combination thereof, wherein the presence of the one or more signatures in Table 2 in the sample relative to a corresponding sample without the one or more signatures in Table 2, is indicative of disease-specific survival, disease-free interval, progression-free interval, progression-free survival or overall survival in the patient. In one embodiment, the presence of the one or more mutational signatures is indicative of response to therapy. In one embodiment, the presence of the one or more mutational signatures is indicative of a need for therapy. In one embodiment, the therapy is radiotherapy. In one embodiment, the therapy is chemotherapy. In one embodiment, the therapy is immunotherapy. In one embodiment, the therapy is antibody therapy.

A kit is provided comprising one or more primers or one or more probes specific for detecting the one or more of the mutational signatures in Table 2.

A microarray is provided comprising one or more probes specific for detecting the one or more of the mutational signatures in Table 2.

Further provided is a method to treat cancer in a human, comprising: administering an anti-cancer therapy to a human having a tumor comprising one or more of the mutational signatures in Table 2 that is/are indicative of decreased disease-specific survival, shorter disease-free interval, shorter progression-free interval, shorter progression-free survival or decreased overall survival. In one embodiment, the human has breast cancer (BRCA), bladder cancer (BLCA), colon adenocarcinoma (COAD), brain lower grade glioma (LGG), liver hepatocellular carcinoma (LIHC), ovarian serous cystadenocarcinoma (OV), stomach adenocarcinoma (STAD), uterine corpus endometrial carcinoma (UCEC), skin cutaneous melanoma (SKC), cervical squamous cell carcinoma and endocervical adenocarcinoma (CESC), head and neck squamous cell carcinoma (HNSC), lung squamous cell carcinoma (LUSC) or pancreatic adenocarcinoma (PAAD). In one embodiment, the one or more signatures are selected from SBS1, SBS2, SBS6, SBS10a, SBS10b, SBS13, SBS15, SBS20, SBS26, or SBS30, or any combination thereof. In one embodiment, the therapy is radiotherapy. In one embodiment, the therapy is chemotherapy. In one embodiment, the therapy is immunotherapy. In one embodiment, the therapy is antibody therapy. In one embodiment, the presence of the one or more mutational signatures is detected using a probe, sequencing or nucleic acid amplification, or a combination thereof.

The invention will be described by the following non-limiting examples.

### Example 1

Mutational signatures, which act as fingerprints of exogenous or endogenous DNA damage, have been associated with cancer incidence, but their influence on cancer outcomes is less clear. It was determined whether single base substitution signatures influenced disease-specific survival (DSS). Exome sequencing data from The Cancer Genome Atlas was utilized. Inclusion was restricted to tumors with at least 50 DSS events. Standard algorithms for mutational signature assessment were applied, and tumor mutational burden (TMB) was evaluated. Cox proportional hazard survival models were fit with adjustment for clinical factors, and hazard ratios and 95% confidence intervals (CI) were calculated. Improvement in model fit with addition of signatures and TMB was quantified using the concordance index (c-index).

### Methods

Tumor exome sequencing data were derived from The Cancer Genome Atlas (TCGA) (Weinstein et al., 2013). Description of cancer patient recruitment, follow-up, and ascertainment of disease outcomes has been published previously (Liu et al., 2018). TCGA somatic mutation data of 10,179 patients (reference genome GRCh38) from 33 cancer types were downloaded from the Genomic Data Commons. Eligible cancers for in-depth analysis were those with at least 50 disease-specific survival (DSS) events, and only TCGA participants with DSS outcomes that were deemed appropriately defined (Liu et al., 2018) were retained. FIGS. 7-16 include data from all 33 cancers, all 49 signatures, and the following measures of survival: overall survival, disease-free interval, disease-specific survival, progression-free survival, and progression-free interval. Stage 0 cancers were also omitted, and those missing stage were excluded from stage-specific analyses. The cancer type abbreviations, full name, and detailed sample size are available in Table 1 and below. The probability matrix for 49 established COSMIC reference mutational signatures (v3) was downloaded from Synapse Documentation (https://www.synapse.org/#ISynapse:syn11738319)).

### Signature Identification

The association of mutational signatures with endogenous processes or exogenous mutagens has been described in several publications (Alexandrov et al., 2020; Alexandrov et al., 2013; Alexandrov et al., 2014; Gerstung et al., 2020; Catalogue of Somatic Mutations in Cancer (https://cancer.sanger.ac.uk/cosmic). A catalog of 96 three-nucleotide motifs that surround the mutational focus (one upstream nucleotide+mutation site+one downstream nucleotide site), and derived frequency tables of this motif catalog for each involved patient, was formalized. A computational function from R package MutationalPatterns (Blokzijl et al., 2018) was leveraged to fit the patient mutational motif frequency tables to the reference mutational signatures while requiring the coefficients, i.e., signature-to-patient contribution strengths, be non-negative values. The estimated coefficients came out in the form of a 96-by-10,179 matrix of non-negative values.

### Statistical Analysis

Disease-specific survival (DSS) was one outcome of interest. Thus, disease-specific mortality was counted as an event, and deaths from other causes (competing risks) and loss-to-follow up were censored. Time to event (cancer, other cause of death, or end of follow-up period) was calculated by TCGA as days from cancer diagnosis. To correct for disease-free immortal person-time, which is present in the time from diagnosis to recruitment, disease-specific survival time was recalculated. Time from diagnosis to recruitment was incorporated into statistical models as immortal person-time. Mutational signatures were modeled in relationship to survival as continuous or discrete (excluding zero) measures, and also using a single cutpoint determined by maximally selected rank statistics (Hothorn, 2003), employing a restriction that the cutpoint include cell sizes of 5 or greater. The three clinical factors commonly available for all organ sites in TCGA data, age at diagnosis, sex, and stage, were included in all analyses. Cox proportional hazards models were fit for DSS, estimating hazard ratios (HR) and 95% confidence intervals (CI). The proportional hazards assumption was verified by Schoenfeld residuals. To further establish the nature of the relationship between mutational signatures and DSS, it was also determined whether stage at diagnosis differed according to signature, comparing Stage I to Stage III/IV disease, using the measures and adjustment factors employed in Cox regression, but quantifying relative risks (RR) and 95% CI.

Models were comparted that included only diagnosis age, sex, tumor grade, and disease stage (baseline model) to a model that also included all mutational signatures significantly associated with survival, and also to a third model that incorporated in addition a measure of tumor mutational burden (TMB). A concordance statistic (c-statistic (Harrell et al., 1996), commonly known as c-index) was calculated to compare improvement in model fit with sequential incorporation of these measures. The difference in c-index between the baseline and other models was assessed. Tumor mutational burden was quantified using nonsynonomous somatic mutations and evaluated as both a continuous variable and using cutpoints derived using maximally selected rank statistics (Hothorn 2003). A two-tailed p-value of <0.05, after adjustment for clinical factors and consideration of the false discovery rate, (FDR) (Benjamini et al., 2001) was considered significant.

### Results

Of solid tumors, 14 organ sites, constituting 16 distinct pathological entities or cancer types, and 6790 patients were included in the in-depth analysis, as these had at least 50 DSS events (Table 1). (GBM was later excluded due to less than 50 events after consideration of immortal person time). Among cancer types, the number of included patients ranged from 170 for pancreatic adenocarcinoma (PAAD) to over 950 for breast carcinoma (BRCA; n=965). Mean age of included patients ranged from 42.9 years (Low Grade Glioma (LGG)) to 68.1 (Bladder Carcinosarcoma (BLCA)). Signatures of 49 distinct mutational patterns of single base substitution (SBS) were examined in relationship to DSS (Catalogue of Somatic Mutations in Cancer (https://cancer.sanger.ac.uk/cosmic). The proportional hazards assumption was violated for 17 signature-cancer combinations in total (these combinations are SBS2BRCA SBS6BRCA SBS32BRCA, SBS35CESC SBS36CESC, SBS17bCOAD SBS29COAD, SBS15HNSC, SBS14LIHC SBS36LIHC, SBS24LUAD SBS37LUAD, SBS39LUSC, SBS190V SBS10V SBS300V, SBS17aPAAD SBS24PAAD, SBS7aSARC SBS13SARC SBS85SARC) thus HR are presented separately for survival times in which hazards were proportional.

TABLE 1
Selected characteristics of participants in The Cancer
Genome Atlas (TCGA) followed for cancer survival

| | Cancer | Events | | | Stage |
|---|---|---|---|---|---|
| Cancer | Cases | (DSS) | Age | Female | 1/2/3/4/unknown |
| Abbreviation | (n) | (n) | (mean) | (%) | (n) |
| BLCA | 393 | 123 | 68.0 | 25.8 | 2/130/132/129/2 |
| BRCA | 917 | 72 | 58.0 | 98.8 | 162/537/199/19/19 |
| CESC | 268 | 50 | 47.9 | 100 | 0/0/0/0/268 |
| COAD | 352 | 56 | 66.3 | 48.1 | 60/135/101/56/10 |
| HNSC | 415 | 129 | 60.9 | 26.1 | 25/66/73/251/64 |
| LGG | 487 | 111 | 43.2 | 25.0 | 0/0/0/0/487 |
| LIHC | 323 | 75 | 59.3 | 32.3 | 163/78/79/3/21 |
| LUAD | 446 | 108 | 65.0 | 54.1 | 249/106/70/21/7 |
| LUSC | 415 | 87 | 67.0 | 25.1 | 205/135/68/7/4 |
| OV | 339 | 167 | 59.5 | 100 | 0/0/0/0/339 |
| PAAD | 163 | 77 | 64.6 | 44.6 | 16/138/4/5/3 |
| SARC | 232 | 71 | 60.5 | 54.7 | 0/0/0/0/232 |
| SKCM | 409 | 186 | 58.1 | 38.3 | 85/137/165/22/35 |
| STAD | 366 | 98 | 65.1 | 34.8 | 48/122/164/32/11 |
| UCEC | 521 | 57 | 63.9 | 100 | 0/0/0/0/525 |

*Note:
Case counts reflect only participants who had disease specific survival (DSS) information available, and were restricted to cancers with a minimum of n = 50 DSS events, thus constitute less than the full census of cancer patients enrolled in TCGA. In addition, cases were omitted if a DSS event occurred before or at consent.

### Abbreviation Key

DSS Disease-specific survival
BLCA Bladder Urothelial Carcinoma
BRCA Breast invasive carcinoma
CESC Cervical squamous cell carcinoma and endocervical adenocarcinoma
COAD Colon adenocarcinoma
GBM Glioblastoma multiforme
HNSC Head and Neck squamous cell carcinoma
LGG Brain Lower Grade Glioma
LIHC Liver hepatocellular carcinoma
LUAD Lung adenocarcinoma
LUSC Lung squamous cell carcinoma
OV Ovarian serous cystadenocarcinoma
PAAD Pancreatic adenocarcinoma
SARC Sarcoma
SKCM Skin Cutaneous Melanoma
STAD Stomach adenocarcinoma
UCEC Uterine Corpus Endometrial Carcinoma
   Signatures Associated with DSS

Individuals with specific tumor mutational signatures had altered DSS that exceeded the FDR threshold in all included cancers except Lung squamous carcinoma (LUSC) (L, and Lung Adenocarcinoma (LUAD). Table 2 While results derived using continuous or discrete measures often supported others, only those relationships identified using a single cutpoint and evaluated using hazard ratios are described below.

TABLE 2A
Mutational signatures related to disease-specific survival (DSS), according to each cancer.
Restricted to cancers with DSS events > n = 50, and binary cutpoints with cell sizes > n = 5.

| | | | Continuous Signature Results | | | Discrete Signature Results | | |
|---|---|---|---|---|---|---|---|---|
| Cancer | Sig | Signature Label | HazardRatio | HRLowerCL | HRUpperCL | HazardRatio | HRLowerCL | HRUpperCL |
| BLCA | SBS1 | Endogenous clock-like age | 0.981493 | 0.965658 | 0.997588 | 0.978894 | 0.961871 | 0.996219 |
| BLCA | SBS2 | Endogenous Apobec3a | 0.995605 | 0.992804 | 0.998415 | 0.995588 | 0.992723 | 0.998462 |
| BLCA | SBS4 | Tobacco | 0.994808 | 0.979577 | 1.010277 | 0.987826 | 0.967918 | 1.008143 |
| BLCA | SBS6 | Mismatchrepair | 0.992654 | 0.973406 | 1.012282 | 0.987026 | 0.96299 | 1.011661 |
| BLCA | SBS7a | UV | 0.975196 | 0.962309 | 0.988256 | 0.975966 | 0.960936 | 0.991232 |
| BLCA | SBS7b | UV | 1.004589 | 0.979767 | 1.030041 | 0.957612 | 0.916433 | 1.00064 |
| BLCA | SBS7c | UV | 0.95231 | 0.833246 | 1.088387 | 0.928793 | 0.774834 | 1.113345 |
| BLCA | SBS7d | UV | 0.986684 | 0.84057 | 1.158196 | 0.958651 | 0.741983 | 1.238589 |
| BLCA | SBS10a | Polymerase epsilon hyperm [symbol] | 0.996471 | 0.970314 | 1.023333 | 0.820747 | 0.559875 | 1.203172 |
| BLCA | SBS10b | Polymerase epsilon mutati [symbol] | 0.990042 | 0.982087 | 0.998063 | 0.990108 | 0.981636 | 0.998652 |
| BLCA | SBS12 | Liver cancer | 0.984294 | 0.952718 | 1.016917 | 0.97669 | 0.937629 | 1.017377 |
| BLCA | SBS13 | Endogenous apobec | 0.995799 | 0.993197 | 0.998408 | 0.995732 | 0.993059 | 0.998413 |
| BLCA | SBS14 | Mismatchrepair + PolEpsil | 0.989273 | 0.924448 | 1.058645 | 0.958284 | 0.877646 | 1.046331 |
| BLCA | SBS15 | Mismatchrepair | 0.993146 | 0.974565 | 1.012081 | 0.99451 | 0.977124 | 1.012206 |
| BLCA | SBS16 | Unknown | 0.967939 | 0.927363 | 1.010291 | 0.940811 | 0.889059 | 0.995576 |
| BLCA | SBS17a | Unknown | 0.947236 | 0.855365 | 1.048975 | 0.950378 | 0.831058 | 1.08683 |
| BLCA | SBS17b | Unknown | 0.972663 | 0.906556 | 1.043592 | 0.92679 | 0.837447 | 1.025664 |
| BLCA | SBS18 | ReactiveOxy | 1.058487 | 1.01784 | 1.100757 | 1.081669 | 1.009753 | 1.158707 |
| BLCA | SBS19 | Unknown | 0.986609 | 0.960585 | 1.013337 | 0.994664 | 0.964713 | 1.025546 |
| BLCA | SBS20 | Mismatchrepair + POLD1mut | 0.945328 | 0.864764 | 1.033398 | 0.972045 | 0.882128 | 1.071127 |
| BLCA | SBS21 | Mismatchrepair | 1.033462 | 0.966284 | 1.10531 | 1.079738 | 0.998225 | 1.167908 |
| BLCA | SBS22 | Aristolochic acid | 0.919483 | 0.858426 | 0.984883 | 0.894661 | 0.819573 | 0.976629 |
| BLCA | SBS23 | Unknown | 1.073833 | 1.011831 | 1.139634 | 1.026398 | 0.943321 | 1.116792 |
| BLCA | SBS24 | Aflatoxin | 0.992215 | 0.968691 | 1.016311 | 0.992395 | 0.963268 | 1.022403 |
| BLCA | SBS25 | Chemotherapy | 1.036842 | 1.007281 | 1.06727 | 1.164547 | 1.011244 | 1.341091 |
| BLCA | SBS26 | Mismatchrepair | 0.984583 | 0.96668 | 1.002819 | 0.987106 | 0.967776 | 1.006821 |
| BLCA | SBS28 | Unknown | 0.988981 | 0.937673 | 1.043096 | 0.962429 | 0.846471 | 1.094273 |
| BLCA | SBS29 | Tobacco chewing in Hollst | 0.99986 | 0.978105 | 1.022099 | 0.995 | 0.967392 | 1.023396 |
| BLCA | SBS3 | Homologous repair | 1.000806 | 0.979675 | 1.022394 | 1.002676 | 0.97274 | 1.033532 |
| BLCA | SBS30 | BER repair-NTHL1mut | 0.991043 | 0.975477 | 1.006859 | 0.970452 | 0.949034 | 0.992354 |
| BLCA | SBS31 | PLChemotherapy | 1.019775 | 0.987932 | 1.052645 | 1.000299 | 0.955673 | 1.04701 |
| BLCA | SBS32 | AZA Chemotherapy | 1.019887 | 0.963777 | 1.079265 | 1.015076 | 0.925738 | 1.113036 |
| BLCA | SBS33 | Unknown | 0.98233 | 0.9326 | 1.034712 | 0.979194 | 0.920987 | 1.041079 |
| BLCA | SBS34 | Unknown | 1.347536 | 1.052641 | 1.725045 | 1.982982 | 1.345558 | 2.92237 |
| BLCA | SBS35 | PLChemotherapy | 0.955573 | 0.838447 | 1.089061 | 0.95523 | 0.801349 | 1.138659 |
| BLCA | SBS36 | BERrepair MUTY | 0.916379 | 0.801653 | 1.047525 | 0.932925 | 0.778009 | 1.118688 |
| BLCA | SBS38 | UV | 0.966131 | 0.90372 | 1.032852 | 0.974104 | 0.909813 | 1.042938 |
| BLCA | SBS39 | Unknown | 0.990313 | 0.981808 | 0.998891 | 0.988311 | 0.979036 | 0.997674 |
| BLCA | SBS42 | Haloalkane | 1.009078 | 0.971414 | 1.048202 | 1.011931 | 0.958971 | 1.067815 |
| BLCA | SBS11 | Temozolo Chemotherapy | 1.115308 | 0.939952 | 1.323379 | 0.916931 | 0.58353 | 1.440821 |
| BLCA | SBS37 | Unknown | 0.973116 | 0.910454 | 1.04009 | 0.964407 | 0.883433 | 1.052803 |
| BLCA | SBS40 | Unknown | 0.154996 | 3.6E−92 | 6.67E+89 | | | |
| BLCA | SBS41 | Unknown | 0.857324 | 0.593469 | 1.238489 | 0.00926 | 0 | |
| BLCA | SBS44 | Mismatchrepair | 1.091343 | 1.004711 | 1.185445 | 116.4983 | 0 | |
| BLCA | SBS8 | Unknown | 1.008418 | 0.963024 | 1.055952 | 0.971945 | 0.897014 | 1.053136 |
| BLCA | SBS84 | Actind cytidinedeaminase | 1.046867 | 0.987062 | 1.110296 | 1.019631 | 0.930298 | 1.117542 |
| BLCA | SBS85 | Actind cytidinedeaminase | 0.997559 | 0.818709 | 1.21548 | 1.279738 | 0.882849 | 1.855051 |
| BLCA | SBS9 | PolETA Somatichypermutation | 5.716394 | 1.747194 | 18.70266 | | | |
| BRCA | SBS1 | Endogenous clock-like age | 1.005151 | 0.99511 | 1.015293 | 1.004493 | 0.993762 | 1.015339 |
| BRCA | SBS2 | Endogenous Apobec3a | 1.002796 | 1.001566 | 1.004027 | 1.002653 | 1.001403 | 1.003905 |
| BRCA | SBS4 | Tobacco | 0.99597 | 0.944625 | 1.050105 | 0.948519 | 0.872508 | 1.031152 |
| BRCA | SBS6 | Mismatchrepair | 0.999889 | 0.981811 | 1.018299 | 0.997224 | 0.973373 | 1.02166 |
| BRCA | SBS7a | UV | 1.011896 | 1.003625 | 1.020235 | 1.013435 | 1.005037 | 1.021904 |
| BRCA | SBS7b | UV | 0.997215 | 0.922383 | 1.078118 | 1.013224 | 0.965066 | 1.063785 |
| BRCA | SBS7c | UV | 0.96059 | 0.738479 | 1.249506 | 0.967447 | 0.666592 | 1.404087 |
| BRCA | SBS7d | UV | 1.050139 | 0.877605 | 1.256592 | 1.028746 | 0.784455 | 1.349114 |
| BRCA | SBS8 | Unknown | 0.991645 | 0.939701 | 1.04646 | 0.989926 | 0.92369 | 1.060911 |
| BRCA | SBS10a | Polymerase epsilon hyperm [symbol] | 0.863984 | 0.685736 | 1.088566 | 0.716018 | 0.481665 | 1.064396 |
| BRCA | SBS10b | Polymerase epsilon mutati [symbol] | 1.009437 | 1.000058 | 1.018904 | 1.008503 | 0.998514 | 1.018591 |
| BRCA | SBS11 | Temozolo Chemotherapy | 0.995307 | 0.850184 | 1.165203 | 1.017333 | 0.86021 | 1.203156 |
| BRCA | SBS12 | Liver cancer | 1.043356 | 0.916251 | 1.188092 | 1.079798 | 0.897052 | 1.299773 |
| BRCA | SBS13 | Endogenous apobec | 1.002114 | 1.001172 | 1.003056 | 1.001979 | 1.001029 | 1.002931 |
| BRCA | SBS14 | Mismatchrepair + PolEpsil | 0.820902 | 0.598964 | 1.125076 | 0.889107 | 0.615412 | 1.284524 |
| BRCA | SBS15 | Mismatchrepair | 1.007454 | 0.992186 | 1.022958 | 1.008658 | 0.99341 | 1.024141 |
| BRCA | SBS16 | Unknown | 0.956844 | 0.826918 | 1.107185 | 0.973377 | 0.788042 | 1.2023 |
| BRCA | SBS17a | Unknown | 0.93195 | 0.769594 | 1.128557 | 0.844644 | 0.632671 | 1.127637 |
| BRCA | SBS17b | Unknown | 0.908818 | 0.701891 | 1.176752 | 1.032503 | 0.802341 | 1.32869 |
| BRCA | SBS18 | ReactiveOxy | 0.968731 | 0.859344 | 1.092042 | 0.834336 | 0.675226 | 1.030938 |
| BRCA | SBS19 | Unknown | 0.999075 | 0.951536 | 1.048988 | 0.997492 | 0.947565 | 1.05005 |
| BRCA | SBS21 | Mismatchrepair | 0.841636 | 0.661964 | 1.070074 | 0.852166 | 0.634966 | 1.143661 |
| BRCA | SBS22 | Aristolochic acid | 1.03463 | 0.95187 | 1.124586 | 1.029411 | 0.936566 | 1.131459 |
| BRCA | SBS23 | Unknown | 0.864286 | 0.633178 | 1.179748 | 0.784969 | 0.474318 | 1.299079 |
| BRCA | SBS24 | Aflatoxin | 0.992185 | 0.95253 | 1.033491 | 0.955238 | 0.871652 | 1.046841 |
| BRCA | SBS25 | Chemotherapy | 1.050591 | 0.999666 | 1.104109 | 1.116039 | 1.029008 | 1.21043 |
| BRCA | SBS26 | Mismatchrepair | 0.988276 | 0.927324 | 1.053233 | 0.993173 | 0.952044 | 1.036079 |
| BRCA | SBS28 | Unknown | 0.907025 | 0.612072 | 1.344113 | 0.977119 | 0.556527 | 1.715571 |
| BRCA | SBS29 | Tobacco chewing in Hollst | 1.02975 | 0.993758 | 1.067045 | 1.046227 | 1.009165 | 1.084649 |
| BRCA | SBS20 | Mismatchrepair + POLD1mut | 1.002452 | 0.98298 | 1.022311 | 0.997727 | 0.972327 | 1.02379 |
| BRCA | SBS30 | BER repair-NTHL1mut | 1.0388 | 1.005039 | 1.073694 | 1.038067 | 0.991869 | 1.086415 |
| BRCA | SBS31 | PLChemotherapy | 0.990496 | 0.891841 | 1.100065 | 1.005322 | 0.860674 | 1.17428 |
| BRCA | SBS32 | AZA Chemotherapy | 1.003092 | 0.975355 | 1.031617 | 0.999972 | 0.938482 | 1.065491 |
| BRCA | SBS33 | Unknown | 1.010039 | 0.916964 | 1.112562 | 1.009733 | 0.905516 | 1.125944 |
| BRCA | SBS34 | Unknown | 0.698516 | 0.452543 | 1.078183 | 0.643019 | 0.320215 | 1.291235 |
| BRCA | SBS35 | PLChemotherapy | 0.970123 | 0.854779 | 1.101032 | 0.995447 | 0.851478 | 1.163758 |
| BRCA | SBS36 | BERrepair MUTY | 1.039906 | 0.976171 | 1.107801 | 1.030396 | 0.948717 | 1.119106 |
| BRCA | SBS37 | Unknown | 1.025153 | 0.956303 | 1.098959 | 1.001732 | 0.904579 | 1.109321 |
| BRCA | SBS38 | UV | 0.945386 | 0.758649 | 1.178087 | 0.877788 | 0.624497 | 1.233812 |
| BRCA | SBS39 | Unknown | 1.010101 | 0.999943 | 1.020361 | 1.00834 | 0.997187 | 1.019619 |
| BRCA | SBS42 | Haloalkane | 1.015958 | 0.95234 | 1.083825 | 1.04825 | 0.976674 | 1.125072 |
| BRCA | SBS84 | Actind cytidinedeaminase | 0.829367 | 0.618271 | 1.112536 | 0.89813 | 0.618701 | 1.303761 |
| BRCA | SBS85 | Actind cytidinedeaminase | 1.020952 | 0.84485 | 1.233762 | 0.92979 | 0.689695 | 1.253466 |
| BRCA | SBS3 | Homologous repair | 0.995769 | 0.951626 | 1.041959 | 0.960177 | 0.892286 | 1.033233 |
| BRCA | SBS40 | Unknown | 0.93458 | 0.73552 | 1.187514 | 0.277338 | 0 | |
| BRCA | SBS41 | Unknown | 0.118976 | 0.000695 | 20.38172 | 0.017993 | 0 | |
| BRCA | SBS44 | Mismatchrepair | 1.025657 | 0.966732 | 1.088175 | 1.039621 | 0.948128 | 1.139943 |
| BRCA | SBS9 | PolETA Somatichypermutati [symbol] | 1.321147 | 0.946648 | 1.843802 | 1.812665 | 0.307464 | 10.68665 |
| CESC | SBS1 | Endogenous clock-like age | 0.978954 | 0.955263 | 1.003233 | 0.980983 | 0.95685 | 1.005726 |
| CESC | SBS2 | Endogenous Apobec3a | 0.998277 | 0.995037 | 1.001526 | 0.998384 | 0.995157 | 1.001622 |
| CESC | SBS4 | Tobacco | 0.981276 | 0.923865 | 1.042256 | 0.960829 | 0.787895 | 1.171721 |
| CESC | SBS6 | Mismatchrepair | 0.999093 | 0.99278 | 1.005447 | 0.999029 | 0.991982 | 1.006127 |
| CESC | SBS7a | UV | 0.993287 | 0.980672 | 1.006065 | 0.994859 | 0.981703 | 1.008191 |
| CESC | SBS7b | UV | 0.980414 | 0.915376 | 1.050074 | 0.998493 | 0.982488 | 1.014759 |
| CESC | SBS7c | UV | 1.064032 | 0.892175 | 1.268993 | 1.02342 | 0.787229 | 1.330475 |
| CESC | SBS7d | UV | 1.005493 | 0.841433 | 1.20154 | 1.24957 | 0.931639 | 1.675999 |
| CESC | SBS8 | Unknown | 1.053155 | 1.003737 | 1.105005 | 1.025478 | 0.951272 | 1.105473 |
| CESC | SBS10a | Polymerase epsilon hyperm [symbol] | 0.993174 | 0.942461 | 1.046616 | 0.990191 | 0.912818 | 1.074122 |
| CESC | SBS10b | Polymerase epsilon mutati [symbol] | 0.992011 | 0.97956 | 1.00462 | 0.993024 | 0.980234 | 1.005981 |
| CESC | SBS11 | Temozolo Chemotherapy | 0.842997 | 0.562201 | 1.264038 | 0.002785 | 0 | |
| CESC | SBS12 | Liver cancer | 1.03684 | 0.899382 | 1.195306 | 1.045708 | 0.832922 | 1.312855 |
| CESC | SBS13 | Endogenous apobec | 0.999251 | 0.997057 | 1.00145 | 0.999369 | 0.997231 | 1.001511 |
| CESC | SBS14 | Mismatchrepair + PolEpsil | 0.96964 | 0.877581 | 1.071357 | 0.959805 | 0.839842 | 1.096904 |
| CESC | SBS15 | Mismatchrepair | 0.991113 | 0.970124 | 1.012557 | 0.979463 | 0.93577 | 1.025196 |
| CESC | SBS16 | Unknown | 0.918261 | 0.740065 | 1.139363 | 0.860741 | 0.639592 | 1.158355 |
| CESC | SBS17a | Unknown | 1.068158 | 0.981877 | 1.162022 | 1.077578 | 0.985229 | 1.178582 |
| CESC | SBS17b | Unknown | 1.001765 | 0.959987 | 1.045361 | 0.995787 | 0.94333 | 1.05116 |
| CESC | SBS18 | ReactiveOxy | 1.031284 | 0.966715 | 1.100164 | 1.052225 | 0.937816 | 1.180592 |
| CESC | SBS19 | Unknown | 1.059596 | 0.964393 | 1.164196 | 1.148172 | 0.995342 | 1.324468 |
| CESC | SBS21 | Mismatchrepair | 0.973777 | 0.879401 | 1.078283 | 0.95227 | 0.79585 | 1.139433 |
| CESC | SBS22 | Aristolochic acid | 0.977242 | 0.857979 | 1.113084 | 0.986642 | 0.853398 | 1.140689 |
| CESC | SBS23 | Unknown | 1.077091 | 0.935198 | 1.240512 | 59.57349 | 0 | |
| CESC | SBS24 | Aflatoxin | 1.0268 | 0.985766 | 1.069541 | 1.042163 | 0.990456 | 1.096569 |
| CESC | SBS25 | Chemotherapy | 1.000613 | 0.969628 | 1.032588 | 0.366643 | 0 | |
| CESC | SBS26 | Mismatchrepair | 0.992997 | 0.9593 | 1.027877 | 0.976246 | 0.93362 | 1.020818 |
| CESC | SBS28 | Unknown | 0.904318 | 0.409056 | 1.999216 | 0.290392 | 0.01155 | 7.300808 |
| CESC | SBS29 | Tobacco chewing in Hollst | 0.992533 | 0.926289 | 1.063515 | 1.002401 | 0.916336 | 1.09655 |
| CESC | SBS20 | Mismatchrepair + POLD1mut | 1.008969 | 0.968999 | 1.050589 | 1.000722 | 0.948006 | 1.056369 |
| CESC | SBS30 | BER repair-NTHL1mut | 1.040215 | 0.952702 | 1.135768 | 1.034728 | 0.90886 | 1.178028 |
| CESC | SBS31 | PLChemotherapy | 1.005635 | 0.94218 | 1.073364 | 1.050309 | 0.96385 | 1.144523 |
| CESC | SBS32 | AZA Chemotherapy | 1.00299 | 0.822731 | 1.222744 | 2.504853 | 0.814407 | 7.704118 |
| CESC | SBS33 | Unknown | 1.000529 | 0.981659 | 1.019761 | 1.004855 | 0.986759 | 1.023283 |
| CESC | SBS34 | Unknown | 1.475118 | 0.949296 | 2.292198 | 0.75838 | 0.367714 | 1.564096 |
| CESC | SBS35 | PLChemotherapy | 1.05777 | 0.944929 | 1.184087 | 0.984368 | 0.852715 | 1.136348 |
| CESC | SBS36 | BERrepair MUTY | 0.964674 | 0.854099 | 1.089565 | 0.905482 | 0.730005 | 1.123139 |
| CESC | SBS37 | Unknown | 0.99418 | 0.963591 | 1.02574 | 0.978348 | 0.912799 | 1.048603 |
| CESC | SBS38 | UV | 1.073163 | 0.929041 | 1.239643 | 1.012367 | 0.81805 | 1.252841 |
| CESC | SBS39 | Unknown | 1.001248 | 0.988016 | 1.014657 | 1.005744 | 0.992769 | 1.01889 |
| CESC | SBS39 | Unknown | 1.001248 | 0.988016 | 1.014657 | 1.005744 | 0.992769 | 1.01889 |
| CESC | SBS42 | Haloalkane | 1.204991 | 1.072986 | 1.353236 | 1.195688 | 0.955721 | 1.495907 |
| CESC | SBS84 | Actind cytidinedeaminase | 0.958246 | 0.802558 | 1.144137 | 0.943429 | 0.761538 | 1.168763 |
| CESC | SBS85 | Actind cytidinedeaminase | 1.187077 | 0.748517 | 1.882592 | 16.9875 | 0.76848 | 375.5141 |
| CESC | SBS3 | Homologous repair | 1.020689 | 0.977519 | 1.065767 | 0.122168 | 0 | |
| CESC | SBS40 | Unknown | | | | | | |
| CESC | SBS41 | Unknown | 0.969426 | 0.829908 | 1.132399 | 1656.58 | 0 | |
| CESC | SBS44 | Mismatchrepair | 1.267903 | 0.956554 | 1.680593 | 894.468 | 0 | |
| CESC | SBS9 | PolETA Somatichypermutati [symbol] | 1.072014 | 0.839379 | 1.369125 | 0.774022 | 0.430598 | 1.391346 |
| COAD | SBS1 | Endogenous clock-like age | 1.00023 | 0.998589 | 1.001875 | 1.000277 | 0.99864 | 1.001917 |
| COAD | SBS2 | Endogenous Apobec3a | 0.995976 | 0.964133 | 1.028871 | 1.002065 | 0.977475 | 1.027273 |
| COAD | SBS4 | Tobacco | 0.992313 | 0.966367 | 1.018957 | 1.015139 | 0.984543 | 1.046685 |
| COAD | SBS6 | Mismatchrepair | 1.00053 | 0.999098 | 1.001964 | 1.0005 | 0.998988 | 1.002014 |
| COAD | SBS7a | UV | 0.978869 | 0.917947 | 1.043833 | 0.989746 | 0.916951 | 1.06832 |
| COAD | SBS7b | UV | 0.992435 | 0.968395 | 1.017072 | 0.987307 | 0.955036 | 1.020669 |
| COAD | SBS7c | UV | 0.861032 | 0.722652 | 1.02591 | 0.733211 | 0.558835 | 0.961998 |
| COAD | SBS7d | UV | 0.972274 | 0.88869 | 1.063719 | 0.921281 | 0.759021 | 1.118228 |
| COAD | SBS8 | Unknown | 1.007757 | 0.962574 | 1.055062 | 1.053446 | 0.982435 | 1.129589 |
| COAD | SBS10a | Polymerase epsilon hyperm [symbol] | 1.000455 | 0.999571 | 1.00134 | 1.000322 | 0.999373 | 1.001271 |
| COAD | SBS10b | Polymerase epsilon mutati [symbol] | 1.000443 | 0.999977 | 1.00091 | 1.000394 | 0.999921 | 1.000867 |
| COAD | SBS11 | Temozolo Chemotherapy | 1.012707 | 0.997254 | 1.0284 | 6.451911 | 0.11413 | 364.7335 |
| COAD | SBS12 | Liver cancer | 0.992814 | 0.916086 | 1.075969 | 1.045024 | 0.860883 | 1.268552 |
| COAD | SBS13 | Endogenous apobec | 0.990008 | 0.914754 | 1.071453 | 0.990713 | 0.905006 | 1.084537 |
| COAD | SBS14 | Mismatchrepair + PolEpsil | 1.000637 | 0.993928 | 1.007392 | 0.997327 | 0.985288 | 1.009513 |
| COAD | SBS15 | Mismatchrepair | 1.000411 | 0.999364 | 1.00146 | 1.00025 | 0.99916 | 1.00134 |
| COAD | SBS16 | Unknown | 0.992494 | 0.851271 | 1.157147 | 1.057905 | 0.84366 | 1.326557 |
| COAD | SBS17a | Unknown | 1.029089 | 1.001946 | 1.056967 | 1.031911 | 1.003708 | 1.060907 |
| COAD | SBS17b | Unknown | 1.006838 | 0.99814 | 1.015613 | 1.006412 | 0.997641 | 1.015259 |
| COAD | SBS18 | ReactiveOxy | 1.00927 | 0.987764 | 1.031244 | 1.013097 | 0.987492 | 1.039367 |
| COAD | SBS19 | Unknown | 1.003313 | 0.929518 | 1.082967 | 0.99311 | 0.902581 | 1.092719 |
| COAD | SBS21 | Mismatchrepair | 1.004546 | 0.999862 | 1.009251 | 1.003525 | 0.997984 | 1.009096 |
| COAD | SBS22 | Aristolochic acid | 1.00105 | 0.987504 | 1.014781 | 0.998644 | 0.982396 | 1.01516 |
| COAD | SBS23 | Unknown | 1.003721 | 0.957272 | 1.052424 | 0.988424 | 0.900846 | 1.084515 |
| COAD | SBS24 | Aflatoxin | 1.006731 | 0.983608 | 1.030397 | 1.005823 | 0.978106 | 1.034325 |
| COAD | SBS25 | Chemotherapy | 1.079391 | 1.022887 | 1.139016 | 1.007072 | 0.913725 | 1.109955 |
| COAD | SBS26 | Mismatchrepair | 1.001962 | 0.999211 | 1.00472 | 0.99979 | 0.996276 | 1.003317 |
| COAD | SBS28 | Unknown | 1.001527 | 0.998337 | 1.004728 | 1.00124 | 0.997878 | 1.004613 |
| COAD | SBS29 | Tobacco chewing in Hollst | 0.976737 | 0.945671 | 1.008824 | 0.997083 | 0.960826 | 1.034707 |
| COAD | SBS20 | Mismatchrepair + POLD1mut | 1.001773 | 0.996312 | 1.007265 | 1.001022 | 0.992559 | 1.009557 |
| COAD | SBS30 | BER repair-NTHL1mut | 1.005782 | 0.975678 | 1.036815 | 0.982929 | 0.921481 | 1.048474 |
| COAD | SBS31 | PLChemotherapy | 0.974186 | 0.910914 | 1.041852 | 0.994282 | 0.910341 | 1.085963 |
| COAD | SBS32 | AZA Chemotherapy | 0.955121 | 0.839423 | 1.086765 | 0.979473 | 0.806939 | 1.188896 |
| COAD | SBS33 | Unknown | 1.020055 | 1.004201 | 1.03616 | 1.017277 | 0.999369 | 1.035506 |
| COAD | SBS34 | Unknown | 1.021734 | 0.957263 | 1.090547 | 1.030511 | 0.955226 | 1.11173 |
| COAD | SBS35 | PLChemotherapy | 0.993356 | 0.942994 | 1.046407 | 1.000926 | 0.934312 | 1.072289 |
| COAD | SBS36 | BERrepair MUTY | 1.022147 | 1.006287 | 1.038258 | 1.043678 | 0.991829 | 1.098238 |
| COAD | SBS37 | Unknown | 1.00002 | 0.990424 | 1.009709 | 0.998048 | 0.985468 | 1.010789 |
| COAD | SBS38 | UV | 1.028867 | 0.978915 | 1.081367 | 1.021228 | 0.958884 | 1.087627 |
| COAD | SBS39 | Unknown | 1.003501 | 0.99166 | 1.015483 | 1.004772 | 0.993598 | 1.016071 |
| COAD | SBS42 | Haloalkane | 1.007971 | 1.000321 | 1.01568 | 1.017601 | 1.000163 | 1.035343 |
| COAD | SBS84 | Actind cytidinedeaminase | 0.999 | 0.970908 | 1.027905 | 0.969387 | 0.913587 | 1.028595 |
| COAD | SBS85 | Actind cytidinedeaminase | 0.99257 | 0.930052 | 1.05929 | 1.72825 | 0.70778 | 4.220019 |
| COAD | SBS3 | Homologous repair | 1.032988 | 0.995147 | 1.072268 | 1.751105 | 0 | |
| COAD | SBS40 | Unknown | 2.49E−11 | 0 | | | | |
| COAD | SBS41 | Unknown | 0.994381 | 0.767235 | 1.288774 | 4.76E+28 | 0 | |
| COAD | SBS44 | Mismatchrepair | 1.059697 | 0.927449 | 1.210802 | 0.457852 | 0 | |
| COAD | SBS9 | PolETA Somatichypermutation | 1.035875 | 1.016589 | 1.055526 | 1.05762 | 0.991097 | 1.128609 |
| HNSC | SBS1 | Endogenous clock-like age | 0.991593 | 0.974633 | 1.008849 | 0.993271 | 0.975969 | 1.010881 |
| HNSC | SBS2 | Endogenous Apobec3a | 0.99895 | 0.993735 | 1.004191 | 0.998579 | 0.993064 | 1.004124 |
| HNSC | SBS4 | Tobacco | 0.997272 | 0.993369 | 1.00119 | 0.996046 | 0.991625 | 1.000488 |
| HNSC | SBS6 | Mismatchrepair | 0.99326 | 0.978594 | 1.008145 | 0.993745 | 0.975384 | 1.012453 |
| HNSC | SBS7a | UV | 1.000415 | 0.989516 | 1.011434 | 0.996678 | 0.982526 | 1.011034 |
| HNSC | SBS7a | UV | 1.000415 | 0.989516 | 1.011434 | 0.996678 | 0.982526 | 1.011034 |
| HNSC | SBS7b | UV | 0.982973 | 0.954936 | 1.011833 | | | |
| HNSC | SBS7c | UV | 1.048905 | 0.946442 | 1.162461 | 1.079715 | 0.930373 | 1.253029 |
| HNSC | SBS7d | UV | 0.98962 | 0.901157 | 1.086767 | 1.004581 | 0.9025 | 1.118209 |
| HNSC | SBS8 | Unknown | 1.025664 | 0.993834 | 1.058514 | 1.024545 | 0.971481 | 1.080508 |
| HNSC | SBS10a | Polymerase epsilon hyperm [symbol] | 1.067807 | 0.923518 | 1.234641 | 1.124371 | 0.906569 | 1.394499 |
| HNSC | SBS10b | Polymerase epsilon mutati [symbol] | 0.993443 | 0.977987 | 1.009143 | 0.996495 | 0.980857 | 1.012383 |
| HNSC | SBS11 | Temozolo Chemotherapy | 0.859966 | 0.708049 | 1.044477 | 0.880707 | 0.63532 | 1.220873 |
| HNSC | SBS12 | Liver cancer | 0.974568 | 0.912113 | 1.0413 | 0.960975 | 0.878931 | 1.050677 |
| HNSC | SBS13 | Endogenous apobec | 0.998464 | 0.994191 | 1.002755 | 0.998018 | 0.99357 | 1.002487 |
| HNSC | SBS14 | Mismatchrepair + PolEpsil | 0.971889 | 0.865439 | 1.091432 | 0.906288 | 0.728207 | 1.127918 |
| HNSC | SBS15 | Mismatchrepair | 0.983139 | 0.956136 | 1.010904 | 0.990173 | 0.960399 | 1.02087 |
| HNSC | SBS16 | Unknown | 1.017345 | 0.993229 | 1.042046 | 1.014953 | 0.9848 | 1.046029 |
| HNSC | SBS17a | Unknown | 1.008119 | 0.937284 | 1.084308 | 0.997858 | 0.909645 | 1.094627 |
| HNSC | SBS17b | Unknown | 0.996064 | 0.858611 | 1.155521 | 0.970951 | 0.78535 | 1.200414 |
| HNSC | SBS18 | ReactiveOxy | 0.980634 | 0.942708 | 1.020085 | 0.971843 | 0.919007 | 1.027716 |
| HNSC | SBS19 | Unknown | 0.982463 | 0.949241 | 1.016848 | 0.986323 | 0.946011 | 1.028353 |
| HNSC | SBS21 | Mismatchrepair | 0.997224 | 0.974708 | 1.020261 | 0.966282 | 0.876334 | 1.065462 |
| HNSC | SBS22 | Aristolochic acid | 0.96274 | 0.915546 | 1.012367 | 0.977532 | 0.9216 | 1.036858 |
| HNSC | SBS23 | Unknown | 0.984571 | 0.929603 | 1.04279 | 0.910601 | 0.817975 | 1.013716 |
| HNSC | SBS24 | Aflatoxin | 0.997819 | 0.985898 | 1.009884 | 0.991347 | 0.976619 | 1.006297 |
| HNSC | SBS25 | Chemotherapy | 1.00135 | 0.977338 | 1.025951 | 0.986122 | 0.945699 | 1.028273 |
| HNSC | SBS26 | Mismatchrepair | 0.965096 | 0.927984 | 1.003693 | 0.928177 | 0.874629 | 0.985003 |
| HNSC | SBS28 | Unknown | 1.092198 | 0.735927 | 1.620943 | 1.328343 | 0.728783 | 2.421153 |
| HNSC | SBS29 | Tobacco chewing in Hollst | 1.031952 | 1.003785 | 1.060909 | 1.02063 | 0.985194 | 1.057341 |
| HNSC | SBS20 | Mismatchrepair + POLD1mut | 0.949256 | 0.869078 | 1.036832 | 0.93422 | 0.8196 | 1.064869 |
| HNSC | SBS30 | BER repair-NTHL1mut | 0.989794 | 0.969022 | 1.011012 | 0.9819 | 0.956164 | 1.008328 |
| HNSC | SBS31 | PLChemotherapy | 1.023428 | 0.998071 | 1.049429 | 1.011066 | 0.976981 | 1.04634 |
| HNSC | SBS32 | AZA Chemotherapy | 1.037123 | 0.984435 | 1.09263 | 1.047686 | 0.971545 | 1.129795 |
| HNSC | SBS33 | Unknown | 0.994522 | 0.941048 | 1.051035 | 0.993138 | 0.934449 | 1.055513 |
| HNSC | SBS34 | Unknown | 1.138389 | 0.958674 | 1.351795 | 1.237414 | 0.932415 | 1.642179 |
| HNSC | SBS35 | PLChemotherapy | 1.006011 | 0.981712 | 1.030911 | 1.002378 | 0.956703 | 1.050233 |
| HNSC | SBS36 | BERrepair MUTY | 1.039448 | 0.98893 | 1.092546 | 1.015131 | 0.938474 | 1.098048 |
| HNSC | SBS38 | UV | 1.008821 | 0.963929 | 1.055803 | 1.024244 | 0.973755 | 1.07735 |
| HNSC | SBS39 | Unknown | 1.000981 | 0.995482 | 1.006509 | 1.001314 | 0.995702 | 1.006958 |
| HNSC | SBS42 | Haloalkane | 1.005441 | 0.948642 | 1.065641 | 0.991758 | 0.912178 | 1.07828 |
| HNSC | SBS84 | Actind cytidinedeaminase | 1.080561 | 0.991173 | 1.17801 | 1.0654 | 0.900856 | 1.259998 |
| HNSC | SBS3 | Homologous repair | 1.071467 | 1.007193 | 1.139843 | 0.980429 | 0.862822 | 1.114067 |
| HNSC | SBS37 | Unknown | 1.004859 | 0.901498 | 1.120072 | 0.856978 | 0.701356 | 1.04713 |
| HNSC | SBS40 | Unknown | 1.276271 | 1.0443 | 1.559771 | 0.165969 | 0 | |
| HNSC | SBS41 | Unknown | 1.010676 | 0.85206 | 1.198818 | 3.15E−09 | 0 | |
| HNSC | SBS44 | Mismatchrepair | 1.091642 | 0.831629 | 1.432948 | 1.79505 | 0.728752 | 4.421543 |
| HNSC | SBS85 | Actind cytidinedeaminase | 1.107256 | 1.006628 | 1.217943 | 1.440022 | 1.181002 | 1.755852 |
| HNSC | SBS9 | PolETA Somatichypermutati [symbol] | 1.694576 | 1.261187 | 2.276893 | 6940.55 | 0 | |
| LGG | SBS1 | Endogenous clock-like age | 1.003849 | 0.998432 | 1.009296 | 1.0037 | 0.998162 | 1.009269 |
| LGG | SBS2 | Endogenous Apobec3a | 0.915614 | 0.703898 | 1.191009 | 0.938527 | 0.63325 | 1.390971 |
| LGG | SBS4 | Tobacco | 0.995277 | 0.895766 | 1.105844 | 0.854104 | 0.679058 | 1.074273 |
| LGG | SBS6 | Mismatchrepair | 1.010371 | 0.965148 | 1.057713 | 1.006497 | 0.936812 | 1.081365 |
| LGG | SBS7a | UV | 1.020366 | 0.887536 | 1.173074 | 1.0945 | 0.900758 | 1.329913 |
| LGG | SBS7b | UV | 0.980006 | 0.889345 | 1.079909 | 1.063554 | 0.930402 | 1.215763 |
| LGG | SBS7c | UV | 0.795322 | 0.565473 | 1.118596 | 0.845477 | 0.457405 | 1.562798 |
| LGG | SBS7d | UV | 0.955279 | 0.80808 | 1.129293 | 1.158278 | 0.94104 | 1.425665 |
| LGG | SBS8 | Unknown | 1.146936 | 1.016015 | 1.294728 | 0.942425 | 0.775298 | 1.145579 |
| LGG | SBS10a | Polymerase epsilon hyperm [symbol] | 1.025229 | 0.751977 | 1.397774 | 1.188348 | 0.714198 | 1.977282 |
| LGG | SBS10b | Polymerase epsilon mutati [symbol] | 1.09562 | 1.004562 | 1.194933 | 1.104198 | 0.992366 | 1.228632 |
| LGG | SBS11 | Temozolo Chemotherapy | 1.08679 | 0.892234 | 1.323771 | 1.064778 | 0.792431 | 1.430727 |
| LGG | SBS12 | Liver cancer | 0.838783 | 0.743406 | 0.946397 | 0.731309 | 0.596599 | 0.896435 |
| LGG | SBS13 | Endogenous apobec | 1.066058 | 0.992696 | 1.144841 | 1.061794 | 0.977836 | 1.152961 |
| LGG | SBS14 | Mismatchrepair + PolEpsil | 1.330013 | 0.905882 | 1.952721 | 2.127037 | 0.896995 | 5.043823 |
| LGG | SBS15 | Mismatchrepair | 0.983168 | 0.94874 | 1.018845 | 0.996205 | 0.95165 | 1.042846 |
| LGG | SBS16 | Unknown | 0.938527 | 0.852605 | 1.033108 | 0.934937 | 0.821165 | 1.064472 |
| LGG | SBS17a | Unknown | 1.008278 | 0.841953 | 1.20746 | 0.986221 | 0.765878 | 1.269958 |
| LGG | SBS17b | Unknown | 0.982815 | 0.699456 | 1.380968 | 1.14253 | 0.504078 | 2.58963 |
| LGG | SBS18 | ReactiveOxy | 1.083014 | 0.596079 | 1.967724 | 1.500636 | 0.376674 | 5.978409 |
| LGG | SBS19 | Unknown | 0.965009 | 0.877934 | 1.060719 | 1.048148 | 0.896585 | 1.225333 |
| LGG | SBS21 | Mismatchrepair | 0.878558 | 0.74484 | 1.036281 | 0.979436 | 0.773813 | 1.239698 |
| LGG | SBS22 | Aristolochic acid | 0.98358 | 0.832629 | 1.161897 | 0.986017 | 0.790459 | 1.229957 |
| LGG | SBS23 | Unknown | 0.939589 | 0.829053 | 1.064864 | 1.112411 | 0.906434 | 1.365195 |
| LGG | SBS24 | Aflatoxin | 1.009456 | 0.922938 | 1.104085 | 1.042299 | 0.916618 | 1.185214 |
| LGG | SBS25 | Chemotherapy | 2.128056 | 1.443883 | 3.13642 | 4.105155 | 0.867341 | 19.42985 |
| LGG | SBS26 | Mismatchrepair | 0.960482 | 0.853846 | 1.080434 | 0.946073 | 0.795843 | 1.124662 |
| LGG | SBS28 | Unknown | 1.129441 | 0.806455 | 1.581782 | 1.486521 | 0.746325 | 2.960837 |
| LGG | SBS29 | Tobacco chewing in Hollst | 1.116807 | 0.931281 | 1.339292 | 1.144964 | 0.836561 | 1.56706 |
| LGG | SBS20 | Mismatchrepair + POLD1mut | 1.150864 | 0.855777 | 1.547703 | 1.247044 | 0.757096 | 2.054058 |
| LGG | SBS30 | BER repair-NTHL1mut | 1.10099 | 1.000954 | 1.211023 | 1.098096 | 0.949295 | 1.270223 |
| LGG | SBS31 | PLChemotherapy | 1.049579 | 0.99773 | 1.104121 | 1.069781 | 0.999307 | 1.145224 |
| LGG | SBS32 | AZA Chemotherapy | 1.07512 | 0.944991 | 1.223168 | 1.132795 | 0.931515 | 1.377568 |
| LGG | SBS33 | Unknown | 1.089641 | 0.941863 | 1.260606 | 1.191672 | 1.001509 | 1.417942 |
| LGG | SBS34 | Unknown | 0.817269 | 0.524693 | 1.272989 | 0.496233 | 0.199116 | 1.236702 |
| LGG | SBS35 | PLChemotherapy | 1.080439 | 0.871383 | 1.339651 | 0.996577 | 0.725136 | 1.369625 |
| LGG | SBS36 | BERrepair MUTY | 1.035687 | 0.835756 | 1.283446 | 1.208931 | 0.815667 | 1.791803 |
| LGG | SBS37 | Unknown | 0.987466 | 0.873641 | 1.116121 | 0.967988 | 0.801319 | 1.169322 |
| LGG | SBS38 | UV | 0.949489 | 0.718749 | 1.254303 | 0.947355 | 0.590446 | 1.520008 |
| LGG | SBS39 | Unknown | 1.004678 | 0.976534 | 1.033633 | 1.000072 | 0.965756 | 1.035607 |
| LGG | SBS42 | Haloalkane | 0.973503 | 0.89247 | 1.061892 | 1.048601 | 0.88627 | 1.240666 |
| LGG | SBS84 | Actind cytidinedeaminase | 0.976813 | 0.84591 | 1.127972 | 1.059257 | 0.857185 | 1.308965 |
| LGG | SBS85 | Actind cytidinedeaminase | 0.816204 | 0.549722 | 1.211864 | 0.38743 | 0.139737 | 1.074177 |
| LGG | SBS3 | Homologous repair | 1.177467 | 0.995106 | 1.393247 | 1.042015 | 0.631358 | 1.71978 |
| LGG | SBS40 | Unknown | 0.001136 | 0 | | | | |
| LGG | SBS41 | Unknown | 0.468142 | 0.081875 | 2.676735 | 4.22E+20 | 0 | |
| LGG | SBS44 | Mismatchrepair | 1.106821 | 0.829875 | 1.47619 | 1.342105 | 0.811105 | 2.220731 |
| LGG | SBS9 | PolETA Somatichypermutati [symbol] | 1.267519 | 0.989671 | 1.623372 | 343147.9 | 0 | |
| LIHC | SBS1 | Endogenous clock-like age | 1.016283 | 1.008607 | 1.024017 | 1.015435 | 1.007567 | 1.023366 |
| LIHC | SBS2 | Endogenous Apobec3a | 1.011933 | 0.914903 | 1.119254 | 1.026614 | 0.906947 | 1.16207 |
| LIHC | SBS3 | Homologous repair | 0.977431 | 0.938783 | 1.01767 | 0.929853 | 0.855759 | 1.010363 |
| LIHC | SBS4 | Tobacco | 1.002796 | 0.993868 | 1.011804 | 1.001872 | 0.990756 | 1.013112 |
| LIHC | SBS6 | Mismatchrepair | 0.961855 | 0.924659 | 1.000547 | 0.995889 | 0.943286 | 1.051424 |
| LIHC | SBS7a | UV | 1.044215 | 0.99658 | 1.094127 | 1.037323 | 0.981377 | 1.096457 |
| LIHC | SBS7b | UV | 0.994797 | 0.948588 | 1.043257 | 0.993853 | 0.932099 | 1.059698 |
| LIHC | SBS7d | UV | 0.994814 | 0.902631 | 1.096413 | 1.020624 | 0.897236 | 1.160981 |
| LIHC | SBS8 | Unknown | 1.008543 | 0.98158 | 1.036247 | 1.008488 | 0.975854 | 1.042213 |
| LIHC | SBS10a | Polymerase epsilon hyperm [symbol] | 0.959996 | 0.783997 | 1.175506 | 1.20572 | 0.875365 | 1.66075 |
| LIHC | SBS10b | Polymerase epsilon mutati [symbol] | 1.041346 | 0.917256 | 1.182222 | 1.063558 | 0.87182 | 1.297463 |
| LIHC | SBS11 | Temozolo Chemotherapy | 0.995816 | 0.843009 | 1.176322 | 0.884131 | 0.642348 | 1.216924 |
| LIHC | SBS12 | Liver cancer | 1.004586 | 0.975238 | 1.034816 | 1.010468 | 0.973897 | 1.048412 |
| LIHC | SBS13 | Endogenous apobec | 1.031801 | 0.908913 | 1.171303 | 1.095449 | 0.936185 | 1.281807 |
| LIHC | SBS14 | Mismatchrepair + PolEpsil | 1.222454 | 1.000806 | 1.49319 | 1.606033 | 1.101588 | 2.341476 |
| LIHC | SBS15 | Mismatchrepair | 1.00744 | 1.003591 | 1.011305 | 1.006297 | 1.002306 | 1.010304 |
| LIHC | SBS16 | Unknown | 0.978565 | 0.942288 | 1.016239 | 0.984608 | 0.944422 | 1.026504 |
| LIHC | SBS17a | Unknown | 1.005486 | 0.952283 | 1.061661 | 1.010323 | 0.956122 | 1.067596 |
| LIHC | SBS17b | Unknown | 1.108402 | 0.97977 | 1.253924 | 1.141418 | 0.974291 | 1.337212 |
| LIHC | SBS18 | ReactiveOxy | 1.028773 | 0.96538 | 1.096328 | 1.047272 | 0.951071 | 1.153204 |
| LIHC | SBS19 | Unknown | 1.015768 | 0.993375 | 1.038666 | 1.019594 | 0.996388 | 1.043341 |
| LIHC | SBS21 | Mismatchrepair | 1.023993 | 1.011558 | 1.03658 | 1.025527 | 1.009658 | 1.041646 |
| LIHC | SBS22 | Aristolochic acid | 0.999747 | 0.994342 | 1.005181 | 0.999197 | 0.992677 | 1.005759 |
| LIHC | SBS23 | Unknown | 1.039039 | 0.949307 | 1.137253 | 1.060356 | 0.909066 | 1.236823 |
| LIHC | SBS24 | Aflatoxin | 1.016459 | 1.008335 | 1.024648 | 1.016213 | 1.007366 | 1.025137 |
| LIHC | SBS25 | Chemotherapy | 0.998161 | 0.988939 | 1.00747 | 0.99754 | 0.988956 | 1.006198 |
| LIHC | SBS26 | Mismatchrepair | 1.00457 | 0.996143 | 1.013069 | 1.005281 | 0.995934 | 1.014715 |
| LIHC | SBS28 | Unknown | 0.980223 | 0.802408 | 1.197441 | 1.021428 | 0.821169 | 1.270525 |
| LIHC | SBS29 | Tobacco chewing in Hollst | 0.96731 | 0.900496 | 1.039081 | 0.968794 | 0.881732 | 1.064452 |
| LIHC | SBS20 | Mismatchrepair + POLD1mut | 1.080048 | 0.887146 | 1.314895 | 0.847351 | 0.602369 | 1.191966 |
| LIHC | SBS30 | BER repair-NTHL1mut | 0.984772 | 0.926955 | 1.046194 | 0.977465 | 0.88697 | 1.077193 |
| LIHC | SBS31 | PLChemotherapy | 0.994002 | 0.942432 | 1.048393 | 0.962134 | 0.874155 | 1.058969 |
| LIHC | SBS32 | AZA Chemotherapy | 0.909992 | 0.789362 | 1.049057 | 1.079981 | 0.871324 | 1.338605 |
| LIHC | SBS33 | Unknown | 0.976716 | 0.908711 | 1.04981 | 0.983237 | 0.923436 | 1.04691 |
| LIHC | SBS34 | Unknown | 0.990085 | 0.817129 | 1.19965 | 0.820617 | 0.628429 | 1.07158 |
| LIHC | SBS35 | PLChemotherapy | 1.002977 | 0.970841 | 1.036178 | 0.994215 | 0.953368 | 1.036812 |
| LIHC | SBS36 | BERrepair MUTY | 0.970027 | 0.890529 | 1.056623 | 1.007188 | 0.882742 | 1.149178 |
| LIHC | SBS37 | Unknown | 0.959163 | 0.853197 | 1.07829 | 0.901124 | 0.701812 | 1.15704 |
| LIHC | SBS38 | UV | 1.061189 | 0.965564 | 1.166284 | 1.064031 | 0.928562 | 1.219264 |
| LIHC | SBS39 | Unknown | 1.019986 | 0.998034 | 1.04242 | 1.028696 | 1.003093 | 1.054953 |
| LIHC | SBS42 | Haloalkane | 1.009355 | 0.974057 | 1.045933 | 1.026193 | 0.972051 | 1.083351 |
| LIHC | SBS85 | Actind cytidinedeaminase | 1.084205 | 1.034136 | 1.136697 | 6.574555 | 0 | |
| LIHC | SBS40 | Unknown | 0.921766 | 0.745197 | 1.140172 | 0.025331 | 0 | |
| LIHC | SBS41 | Unknown | 0.527926 | 0.019908 | 13.99937 | 0.002892 | 0 | |
| LIHC | SBS44 | Mismatchrepair | 1.052659 | 0.923152 | 1.200335 | 0.609617 | 0.328528 | 1.131207 |
| LIHC | SBS7c | UV | 0.729627 | 0.5366 | 0.992089 | 0.332377 | 0.174715 | 0.63231 |
| LIHC | SBS84 | Actind cytidinedeaminase | 1.021352 | 0.969342 | 1.076153 | 1.021525 | 0.919905 | 1.134372 |
| LIHC | SBS9 | PolETA Somatichypermutati [symbol] | 0.602594 | 0.210971 | 1.72118 | 0.000265 | 0 | |
| LUAD | SBS1 | Endogenous clock-like age | 0.991606 | 0.971697 | 1.011923 | 0.997153 | 0.97674 | 1.017992 |
| LUAD | SBS2 | Endogenous Apobec3a | 1.000465 | 0.993613 | 1.007364 | 1.000353 | 0.992981 | 1.00778 |
| LUAD | SBS3 | Homologous repair | 1.016873 | 0.979727 | 1.055427 | 1.075249 | 0.96686 | 1.195788 |
| LUAD | SBS4 | Tobacco | 0.999642 | 0.998594 | 1.000691 | 0.999325 | 0.998144 | 1.000509 |
| LUAD | SBS6 | Mismatchrepair | 1.004702 | 0.99913 | 1.010304 | 1.003803 | 0.997766 | 1.009877 |
| LUAD | SBS7a | UV | 1.012503 | 0.996506 | 1.028756 | 1.012011 | 0.993946 | 1.030404 |
| LUAD | SBS7b | UV | 0.999109 | 0.979756 | 1.018845 | 0.998243 | 0.974708 | 1.022347 |
| LUAD | SBS7c | UV | 0.699439 | 0.495535 | 0.987246 | 0.854917 | 0.533956 | 1.368808 |
| LUAD | SBS7d | UV | 0.940897 | 0.840633 | 1.05312 | 0.946453 | 0.784986 | 1.141131 |
| LUAD | SBS8 | Unknown | 0.997129 | 0.887829 | 1.119884 | 1.047879 | 0.833096 | 1.318036 |
| LUAD | SBS10a | Polymerase epsilon hyperm [symbol] | 0.896747 | 0.800497 | 1.00457 | 0.89426 | 0.764899 | 1.045499 |
| LUAD | SBS10b | Polymerase epsilon mutati [symbol] | 1.018248 | 0.993011 | 1.044126 | 1.011561 | 0.98147 | 1.042574 |
| LUAD | SBS11 | Temozolo Chemotherapy | 1.02779 | 0.899817 | 1.173963 | 0.988366 | 0.757475 | 1.289637 |
| LUAD | SBS12 | Liver cancer | 1.051742 | 1.004895 | 1.100774 | 1.040364 | 0.976105 | 1.108854 |
| LUAD | SBS13 | Endogenous apobec | 1.000645 | 0.994791 | 1.006535 | 1.000316 | 0.994252 | 1.006417 |
| LUAD | SBS14 | Mismatchrepair + PolEpsil | 0.989883 | 0.940231 | 1.042156 | 0.956518 | 0.887111 | 1.031356 |
| LUAD | SBS15 | Mismatchrepair | 0.999564 | 0.984402 | 1.014959 | 0.998197 | 0.98172 | 1.014951 |
| LUAD | SBS16 | Unknown | 0.978753 | 0.916835 | 1.044853 | 0.985497 | 0.887003 | 1.094929 |
| LUAD | SBS17a | Unknown | 0.948878 | 0.878925 | 1.024399 | 0.952065 | 0.870169 | 1.041669 |
| LUAD | SBS17b | Unknown | 1.068191 | 1.000555 | 1.140399 | 1.043955 | 0.954231 | 1.142116 |
| LUAD | SBS18 | ReactiveOxy | 0.991654 | 0.948153 | 1.037149 | 0.969642 | 0.904192 | 1.03983 |
| LUAD | SBS19 | Unknown | 1.004418 | 0.987106 | 1.022034 | 1.009423 | 0.991512 | 1.027657 |
| LUAD | SBS21 | Mismatchrepair | 1.008957 | 0.990447 | 1.027813 | 1.016535 | 0.99925 | 1.03412 |
| LUAD | SBS22 | Aristolochic acid | 0.989742 | 0.960805 | 1.019551 | 0.995219 | 0.959505 | 1.032261 |
| LUAD | SBS23 | Unknown | 1.012143 | 0.932725 | 1.098323 | 1.034052 | 0.917819 | 1.165004 |
| LUAD | SBS24 | Aflatoxin | 0.995095 | 0.98871 | 1.001521 | 0.994623 | 0.987479 | 1.00182 |
| LUAD | SBS25 | Chemotherapy | 1.019513 | 0.999485 | 1.039942 | 1.008303 | 0.979865 | 1.037566 |
| LUAD | SBS26 | Mismatchrepair | 0.997977 | 0.982877 | 1.01331 | 0.998994 | 0.983327 | 1.014909 |
| LUAD | SBS28 | Unknown | 1.265665 | 0.998198 | 1.6048 | 1.251199 | 0.845219 | 1.852182 |
| LUAD | SBS29 | Tobacco chewing in Hollst | 1.004529 | 0.992557 | 1.016645 | 0.99847 | 0.981081 | 1.016167 |
| LUAD | SBS20 | Mismatchrepair + POLD1mut | 0.986058 | 0.947811 | 1.025849 | 1.010597 | 0.955846 | 1.068485 |
| LUAD | SBS30 | BER repair-NTHL1mut | 0.999545 | 0.9811 | 1.018336 | 1.013959 | 0.991475 | 1.036953 |
| LUAD | SBS31 | PLChemotherapy | 0.880629 | 0.799015 | 0.970579 | 0.89312 | 0.790183 | 1.009466 |
| LUAD | SBS32 | AZA Chemotherapy | 1.007033 | 0.917883 | 1.104841 | 1.043587 | 0.915357 | 1.189779 |
| LUAD | SBS33 | Unknown | 1.02678 | 0.989877 | 1.06506 | 1.035637 | 0.996943 | 1.075834 |
| LUAD | SBS34 | Unknown | 0.81226 | 0.55389 | 1.191149 | 0.673981 | 0.367135 | 1.237284 |
| LUAD | SBS35 | PLChemotherapy | 0.996923 | 0.949822 | 1.046359 | 0.997416 | 0.935663 | 1.063245 |
| LUAD | SBS36 | BERrepair MUTY | 0.98615 | 0.935578 | 1.039456 | 1.003078 | 0.920809 | 1.092697 |
| LUAD | SBS37 | Unknown | 0.957005 | 0.772093 | 1.186203 | 1.135224 | 0.840288 | 1.533679 |
| LUAD | SBS38 | UV | 1.001168 | 0.976504 | 1.026455 | 1.006324 | 0.976905 | 1.036629 |
| LUAD | SBS39 | Unknown | 1.002066 | 0.995395 | 1.008781 | 1.003181 | 0.996228 | 1.010182 |
| LUAD | SBS42 | Haloalkane | 1.012819 | 0.988571 | 1.037662 | 0.994742 | 0.962463 | 1.028103 |
| LUAD | SBS84 | Actind cytidinedeaminase | 1.052462 | 0.92855 | 1.192911 | 0.864712 | 0.707477 | 1.056892 |
| LUAD | SBS40 | Unknown | 0.957959 | 0.616794 | 1.487833 | 0.980896 | 0 | |
| LUAD | SBS41 | Unknown | 1.683135 | 1.069099 | 2.649842 | 1269092 | 0 | |
| LUAD | SBS44 | Mismatchrepair | 0.975844 | 0.725802 | 1.312027 | 0.000283 | 0 | |
| LUAD | SBS85 | Actind cytidinedeaminase | 0.874298 | 0.530535 | 1.440805 | 0.774711 | 0.306602 | 1.957514 |
| LUAD | SBS9 | PolETA Somatichypermutati [symbol] | 1.101917 | 0.890607 | 1.363363 | 0.064549 | 0 | |
| LUSC | SBS1 | Endogenous clock-like age | 0.991197 | 0.972212 | 1.010553 | 0.984712 | 0.962477 | 1.007461 |
| LUSC | SBS2 | Endogenous Apobec3a | 1.002116 | 0.994994 | 1.009288 | 1.001743 | 0.994346 | 1.009196 |
| LUSC | SBS3 | Homologous repair | 0.981184 | 0.945106 | 1.018639 | 0.994945 | 0.957047 | 1.034343 |
| LUSC | SBS4 | Tobacco | 0.999915 | 0.997553 | 1.002283 | 1.000125 | 0.997639 | 1.002619 |
| LUSC | SBS6 | Mismatchrepair | 0.977191 | 0.953053 | 1.001942 | 0.990625 | 0.957451 | 1.024949 |
| LUSC | SBS7a | UV | 0.999011 | 0.995636 | 1.002397 | 0.999078 | 0.995373 | 1.002796 |
| LUSC | SBS7b | UV | 0.999776 | 0.99871 | 1.000842 | 0.999733 | 0.998687 | 1.000781 |
| LUSC | SBS7c | UV | 1.000722 | 0.967763 | 1.034804 | 0.99788 | 0.962885 | 1.034146 |
| LUSC | SBS7d | UV | 0.955215 | 0.855547 | 1.066494 | 0.956939 | 0.829583 | 1.103846 |
| LUSC | SBS8 | Unknown | 0.966303 | 0.913224 | 1.022468 | 0.967334 | 0.893988 | 1.046697 |
| LUSC | SBS10a | Polymerase epsilon hyperm [symbol] | 0.917169 | 0.815143 | 1.031965 | 0.910375 | 0.761926 | 1.087746 |
| LUSC | SBS10b | Polymerase epsilon mutati [symbol] | 0.996799 | 0.974894 | 1.019195 | 1.003524 | 0.979695 | 1.027932 |
| LUSC | SBS11 | Temozolo Chemotherapy | 0.957652 | 0.853616 | 1.074368 | 0.893643 | 0.737044 | 1.083515 |
| LUSC | SBS12 | Liver cancer | 1.013032 | 0.981705 | 1.045358 | 1.027458 | 0.981098 | 1.076009 |
| LUSC | SBS13 | Endogenous apobec | 1.00244 | 0.997299 | 1.007607 | 1.002924 | 0.997736 | 1.008139 |
| LUSC | SBS14 | Mismatchrepair + PolEpsil | 0.805816 | 0.623046 | 1.042203 | 0.679011 | 0.435177 | 1.059467 |
| LUSC | SBS15 | Mismatchrepair | 1.001299 | 0.99961 | 1.002991 | 1.001171 | 0.999406 | 1.002939 |
| LUSC | SBS16 | Unknown | 0.989237 | 0.935955 | 1.045553 | 1.009222 | 0.935091 | 1.089229 |
| LUSC | SBS17a | Unknown | 0.994434 | 0.929337 | 1.064091 | 0.954336 | 0.874956 | 1.040917 |
| LUSC | SBS17b | Unknown | 0.971889 | 0.816446 | 1.156927 | 1.056452 | 0.852644 | 1.308977 |
| LUSC | SBS18 | ReactiveOxy | 0.996575 | 0.977836 | 1.015673 | 0.985092 | 0.950933 | 1.020479 |
| LUSC | SBS19 | Unknown | 0.992181 | 0.964026 | 1.021158 | 0.979169 | 0.942723 | 1.017024 |
| LUSC | SBS21 | Mismatchrepair | 0.922737 | 0.840794 | 1.012666 | 0.938501 | 0.83647 | 1.052977 |
| LUSC | SBS22 | Aristolochic acid | 0.985895 | 0.953844 | 1.019023 | 0.992384 | 0.952876 | 1.03353 |
| LUSC | SBS23 | Unknown | 0.927624 | 0.860772 | 0.999667 | 0.95831 | 0.871718 | 1.053504 |
| LUSC | SBS24 | Aflatoxin | 0.996118 | 0.98708 | 1.00524 | 1.001943 | 0.991854 | 1.012134 |
| LUSC | SBS25 | Chemotherapy | 0.995339 | 0.973929 | 1.01722 | 0.998174 | 0.968468 | 1.028792 |
| LUSC | SBS26 | Mismatchrepair | 0.991372 | 0.973456 | 1.009618 | 0.99415 | 0.974549 | 1.014146 |
| LUSC | SBS28 | Unknown | 1.05417 | 0.832317 | 1.335157 | 0.963042 | 0.668381 | 1.387608 |
| LUSC | SBS29 | Tobacco chewing in Hollst | 1.009665 | 0.993695 | 1.025892 | 1.003673 | 0.98054 | 1.027353 |
| LUSC | SBS20 | Mismatchrepair + POLD1mut | 0.999913 | 0.963835 | 1.037342 | 0.966365 | 0.911504 | 1.024527 |
| LUSC | SBS30 | BER repair-NTHL1mut | 0.994764 | 0.976116 | 1.013769 | 0.992675 | 0.968157 | 1.017814 |
| LUSC | SBS31 | PLChemotherapy | 1.013935 | 0.985649 | 1.043033 | 1.013578 | 0.97575 | 1.052873 |
| LUSC | SBS32 | AZA Chemotherapy | 0.865466 | 0.748815 | 1.000288 | 0.927943 | 0.759204 | 1.134186 |
| LUSC | SBS33 | Unknown | 0.911796 | 0.846568 | 0.982049 | 0.933154 | 0.857408 | 1.015592 |
| LUSC | SBS34 | Unknown | 0.932865 | 0.728844 | 1.193996 | 0.839489 | 0.550551 | 1.280067 |
| LUSC | SBS35 | PLChemotherapy | 1.002425 | 0.983206 | 1.02202 | 1.003724 | 0.978672 | 1.029416 |
| LUSC | SBS36 | BERrepair MUTY | 0.910768 | 0.831744 | 0.9973 | 0.995861 | 0.86879 | 1.141517 |
| LUSC | SBS37 | Unknown | 1.050847 | 0.890773 | 1.239688 | 0.767181 | 0.463904 | 1.268724 |
| LUSC | SBS38 | UV | 0.999008 | 0.985563 | 1.012636 | 0.993808 | 0.972651 | 1.015426 |
| LUSC | SBS39 | Unknown | 0.994402 | 0.985831 | 1.003048 | 0.996171 | 0.987439 | 1.004981 |
| LUSC | SBS42 | Haloalkane | 0.981875 | 0.92829 | 1.038552 | 0.979814 | 0.89133 | 1.077081 |
| LUSC | SBS84 | Actind cytidinedeaminase | 0.997309 | 0.886353 | 1.122156 | 1.169105 | 0.876302 | 1.559745 |
| LUSC | SBS85 | Actind cytidinedeaminase | 1.096666 | 0.819327 | 1.467883 | 0.616003 | 0.227812 | 1.665669 |
| LUSC | SBS40 | Unknown | 0.786358 | 0.307482 | 2.011039 | | | |
| LUSC | SBS41 | Unknown | 1.285615 | 0.713674 | 2.31591 | 1.43E+43 | 0 | |
| LUSC | SBS44 | Mismatchrepair | 0.986026 | 0.906962 | 1.071982 | 1.082898 | 0.833575 | 1.406793 |
| LUSC | SBS9 | PolETA Somatichypermutati [symbol] | 0.989094 | 0.711043 | 1.375877 | 0.006287 | 0 | |
| OV | SBS1 | Endogenous clock-like age | 1.026547 | 1.002186 | 1.0515 | 1.035567 | 1.007606 | 1.064304 |
| OV | SBS2 | Endogenous Apobec3a | 1.019574 | 0.985585 | 1.054735 | 1.008298 | 0.967571 | 1.050739 |
| OV | SBS3 | Homologous repair | 1.000239 | 0.997522 | 1.002963 | 0.998965 | 0.996079 | 1.001859 |
| OV | SBS4 | Tobacco | 0.996729 | 0.977392 | 1.016448 | 1.00226 | 0.979532 | 1.025515 |
| OV | SBS6 | Mismatchrepair | 1.000951 | 0.980591 | 1.021733 | 1.002916 | 0.975717 | 1.030875 |
| OV | SBS7a | UV | 0.972344 | 0.933967 | 1.012299 | 1.013818 | 0.9717 | 1.057762 |
| OV | SBS7b | UV | 0.976475 | 0.934117 | 1.020753 | 0.97733 | 0.914485 | 1.044493 |
| OV | SBS7c | UV | 1.008435 | 0.954931 | 1.064937 | 1.014038 | 0.95668 | 1.074834 |
| OV | SBS7d | UV | 1.009564 | 0.935011 | 1.090061 | 1.104863 | 0.996822 | 1.224613 |
| OV | SBS8 | Unknown | 0.979682 | 0.962208 | 0.997472 | 0.973229 | 0.950479 | 0.996524 |
| OV | SBS9 | PolETA Somatichypermutati [symbol] | 0.958239 | 0.901071 | 1.019034 | 0.939082 | 0.846986 | 1.041192 |
| OV | SBS10a | Polymerase epsilon hyperm [symbol] | 1.062548 | 0.929745 | 1.21432 | 1.119132 | 0.919587 | 1.361977 |
| OV | SBS10b | Polymerase epsilon mutati [symbol] | 1.046007 | 0.996088 | 1.098427 | 1.042212 | 0.979108 | 1.109383 |
| OV | SBS11 | Temozolo Chemotherapy | 1.004428 | 0.943991 | 1.068734 | 0.94103 | 0.838699 | 1.055847 |
| OV | SBS12 | Liver cancer | 0.954846 | 0.900589 | 1.012372 | 0.943516 | 0.871163 | 1.021879 |
| OV | SBS13 | Endogenous apobec | 0.98256 | 0.945669 | 1.02089 | 1.001315 | 0.965724 | 1.038218 |
| OV | SBS14 | Mismatchrepair + PolEpsil | 0.993476 | 0.938498 | 1.051674 | 0.972651 | 0.890846 | 1.061969 |
| OV | SBS15 | Mismatchrepair | 0.982126 | 0.948768 | 1.016656 | 0.980292 | 0.93109 | 1.032094 |
| OV | SBS16 | Unknown | 1.045553 | 0.950798 | 1.149751 | 0.995799 | 0.865322 | 1.145949 |
| OV | SBS17a | Unknown | 0.995621 | 0.947498 | 1.046189 | 0.988532 | 0.933211 | 1.047133 |
| OV | SBS17b | Unknown | 0.918168 | 0.800724 | 1.052839 | 0.942803 | 0.791266 | 1.123363 |
| OV | SBS18 | ReactiveOxy | 0.987752 | 0.958416 | 1.017987 | 0.989893 | 0.954982 | 1.02608 |
| OV | SBS19 | Unknown | 0.983705 | 0.952404 | 1.016036 | 0.970778 | 0.93068 | 1.012603 |
| OV | SBS21 | Mismatchrepair | 1.055729 | 0.994724 | 1.120476 | 1.033488 | 0.951939 | 1.122023 |
| OV | SBS22 | Aristolochic acid | 0.997297 | 0.979206 | 1.015722 | 0.993585 | 0.9735 | 1.014084 |
| OV | SBS23 | Unknown | 1.063841 | 0.996963 | 1.135205 | 1.138545 | 1.02811 | 1.260842 |
| OV | SBS24 | Aflatoxin | 1.002533 | 0.984043 | 1.02137 | 0.992236 | 0.968158 | 1.016914 |
| OV | SBS25 | Chemotherapy | 1.002999 | 0.990731 | 1.015418 | 1.002951 | 0.986371 | 1.01981 |
| OV | SBS26 | Mismatchrepair | 1.011969 | 0.962424 | 1.064065 | 0.999196 | 0.930109 | 1.073415 |
| OV | SBS28 | Unknown | 0.982293 | 0.90885 | 1.061671 | 1.01709 | 0.933212 | 1.108506 |
| OV | SBS29 | Tobacco chewing in Hollst | 0.989912 | 0.966259 | 1.014144 | 1.007941 | 0.976743 | 1.040136 |
| OV | SBS20 | Mismatchrepair + POLD1mut | 1.052289 | 1.003188 | 1.103793 | 1.022647 | 0.95241 | 1.098064 |
| OV | SBS30 | BER repair-NTHL1mut | 1.002949 | 0.979482 | 1.026978 | 0.976205 | 0.942402 | 1.01122 |
| OV | SBS31 | PLChemotherapy | 0.99082 | 0.951804 | 1.031436 | 0.959847 | 0.898328 | 1.025578 |
| OV | SBS32 | AZA Chemotherapy | 0.970858 | 0.932558 | 1.010732 | 0.981582 | 0.933592 | 1.032039 |
| OV | SBS33 | Unknown | 1.030349 | 0.954861 | 1.111804 | 1.074246 | 0.975028 | 1.183562 |
| OV | SBS34 | Unknown | 0.994585 | 0.861453 | 1.148292 | 0.989862 | 0.819354 | 1.195854 |
| OV | SBS35 | PLChemotherapy | 0.991791 | 0.959978 | 1.024659 | 0.982316 | 0.93678 | 1.030066 |
| OV | SBS36 | BERrepair MUTY | 1.003391 | 0.966368 | 1.041832 | 0.992728 | 0.943637 | 1.044373 |
| OV | SBS37 | Unknown | 1.001864 | 0.987788 | 1.01614 | 0.989068 | 0.970316 | 1.008182 |
| OV | SBS38 | UV | 0.977508 | 0.91017 | 1.049827 | 1.016307 | 0.951359 | 1.085688 |
| OV | SBS39 | Unknown | 0.996994 | 0.990556 | 1.003474 | 0.997518 | 0.990807 | 1.004274 |
| OV | SBS42 | Haloalkane | 1.013042 | 0.981862 | 1.045213 | 1.01362 | 0.973921 | 1.054938 |
| OV | SBS84 | Actind cytidinedeaminase | 0.977411 | 0.934878 | 1.021879 | 0.958082 | 0.896799 | 1.023553 |
| OV | SBS85 | Actind cytidinedeaminase | 1.100179 | 0.984213 | 1.229809 | 1.186825 | 1.015458 | 1.387112 |
| OV | SBS40 | Unknown | 1.0142 | 0.9857 | 1.043525 | 1.048542 | 0.98826 | 1.112502 |
| OV | SBS41 | Unknown | 0.875853 | 0.64972 | 1.180691 | 1.619845 | 0.905561 | 2.897537 |
| OV | SBS44 | Mismatchrepair | 1.035501 | 0.9565 | 1.121026 | 1.049874 | 0.932963 | 1.181435 |
| PAAD | SBS1 | Endogenous clock-like age | 1.000308 | 0.999008 | 1.001609 | 1.00035 | 0.999054 | 1.001649 |
| PAAD | SBS2 | Endogenous Apobec3a | 0.977058 | 0.891669 | 1.070625 | 0.994446 | 0.896192 | 1.103472 |
| PAAD | SBS3 | Homologous repair | 0.949877 | 0.857614 | 1.052065 | 1.35E−17 | 0 | |
| PAAD | SBS4 | Tobacco | 1.047606 | 0.907042 | 1.209953 | 1.068715 | 0.798265 | 1.430792 |
| PAAD | SBS6 | Mismatchrepair | 1.000243 | 0.999135 | 1.001353 | 1.000225 | 0.999091 | 1.00136 |
| PAAD | SBS7a | UV | 1.009858 | 0.972562 | 1.048584 | 1.009902 | 0.965433 | 1.056418 |
| PAAD | SBS7b | UV | 1.002183 | 0.99171 | 1.012766 | 1.003706 | 0.992227 | 1.015317 |
| PAAD | SBS7c | UV | 0.655251 | 0.405417 | 1.059043 | 0.46211 | 0.18727 | 1.140313 |
| PAAD | SBS7d | UV | 1.004675 | 0.984211 | 1.025565 | 1.012034 | 0.989024 | 1.03558 |
| PAAD | SBS8 | Unknown | 0.606637 | 0.443697 | 0.829415 | 0.4212 | 0.19977 | 0.888067 |
| PAAD | SBS9 | PolETA Somatichypermutati [symbol] | 0.995163 | 0.515077 | 1.922719 | 0.025783 | 0 | |
| PAAD | SBS10a | Polymerase epsilon hyperm [symbol] | 1.00185 | 0.993517 | 1.010253 | 1.002762 | 0.993833 | 1.011772 |
| PAAD | SBS10b | Polymerase epsilon mutati [symbol] | 1.000223 | 0.99915 | 1.001297 | | | |
| PAAD | SBS11 | Temozolo Chemotherapy | 1.000275 | 0.998978 | 1.001574 | 1.000991 | 0.999202 | 1.002783 |
| PAAD | SBS12 | Liver cancer | 0.741179 | 0.520938 | 1.054534 | 0.867836 | 0.459897 | 1.637625 |
| PAAD | SBS13 | Endogenous apobec | 1.009346 | 0.944524 | 1.078617 | 1.00254 | 0.931542 | 1.078949 |
| PAAD | SBS14 | Mismatchrepair + PolEpsil | 1.00011 | 0.999614 | 1.000605 | 1.000064 | 0.999505 | 1.000623 |
| PAAD | SBS15 | Mismatchrepair | 1.000075 | 0.999745 | 1.000404 | 1.000093 | 0.999758 | 1.000428 |
| PAAD | SBS16 | Unknown | 1.04916 | 0.912364 | 1.206467 | 1.033182 | 0.83459 | 1.279029 |
| PAAD | SBS17a | Unknown | 1.033874 | 0.967456 | 1.104852 | 1.035804 | 0.962002 | 1.115268 |
| PAAD | SBS17b | Unknown | 1.001553 | 0.99499 | 1.008158 | 1.00086 | 0.993981 | 1.007787 |
| PAAD | SBS18 | ReactiveOxy | 0.974649 | 0.826597 | 1.149218 | 0.722242 | 0.420651 | 1.240064 |
| PAAD | SBS19 | Unknown | 1.000827 | 0.996916 | 1.004752 | 0.99952 | 0.9953 | 1.003758 |
| PAAD | SBS21 | Mismatchrepair | 1.001329 | 0.994569 | 1.008134 | 1.001648 | 0.994721 | 1.008623 |
| PAAD | SBS22 | Aristolochic acid | 0.892572 | 0.717414 | 1.110494 | 0.916449 | 0.684545 | 1.226915 |
| PAAD | SBS23 | Unknown | 1.199069 | 0.841878 | 1.707809 | 1.652299 | 0.622428 | 4.386198 |
| PAAD | SBS24 | Aflatoxin | 1.000522 | 0.998286 | 1.002763 | 1.000487 | 0.998126 | 1.002854 |
| PAAD | SBS25 | Chemotherapy | 1.288326 | 0.892934 | 1.858798 | 2.07779 | 0.715916 | 6.030335 |
| PAAD | SBS26 | Mismatchrepair | 1.000912 | 0.996958 | 1.004881 | 1.000577 | 0.99643 | 1.004742 |
| PAAD | SBS28 | Unknown | 1.003834 | 0.987779 | 1.02015 | 1.011062 | 0.99112 | 1.031404 |
| PAAD | SBS29 | Tobacco chewing in Hollst | 0.998108 | 0.894236 | 1.114046 | 0.977709 | 0.84216 | 1.135077 |
| PAAD | SBS20 | Mismatchrepair + POLD1mut | 1.163218 | 0.909642 | 1.487482 | 1.0696 | 0.490369 | 2.333025 |
| PAAD | SBS30 | BER repair-NTHL1mut | 1.060864 | 0.925835 | 1.215586 | 1.100808 | 0.896146 | 1.35221 |
| PAAD | SBS31 | PLChemotherapy | 0.917579 | 0.813219 | 1.035331 | 0.935442 | 0.799789 | 1.094103 |
| PAAD | SBS32 | AZA Chemotherapy | 0.929344 | 0.804013 | 1.074212 | 0.925429 | 0.728244 | 1.176007 |
| PAAD | SBS33 | Unknown | 1.003129 | 0.991077 | 1.015328 | 1.000665 | 0.987996 | 1.013496 |
| PAAD | SBS34 | Unknown | 1.252467 | 0.869373 | 1.804373 | 1.068736 | 0.535635 | 2.132414 |
| PAAD | SBS35 | PLChemotherapy | 0.930159 | 0.741202 | 1.167288 | 1.07286 | 0.671546 | 1.713999 |
| PAAD | SBS36 | BERrepair MUTY | 0.977949 | 0.890045 | 1.074536 | 1.00396 | 0.904597 | 1.114238 |
| PAAD | SBS37 | Unknown | 0.950492 | 0.76683 | 1.178143 | 0.979141 | 0.708384 | 1.353386 |
| PAAD | SBS38 | UV | 1.151869 | 0.891832 | 1.487726 | 1.421562 | 0.917286 | 2.203062 |
| PAAD | SBS39 | Unknown | 0.976123 | 0.927275 | 1.027544 | 0.983968 | 0.929601 | 1.041515 |
| PAAD | SBS39 | Unknown | 0.976123 | 0.927275 | 1.027544 | 0.983968 | 0.929601 | 1.041515 |
| PAAD | SBS42 | Haloalkane | 1.050143 | 0.933574 | 1.181267 | 0.940761 | 0.723628 | 1.223048 |
| PAAD | SBS84 | Actind cytidinedeaminase | 0.844745 | 0.564594 | 1.263908 | 0.917852 | 0.305691 | 2.755893 |
| PAAD | SBS85 | Actind cytidinedeaminase | 0.624356 | 0.374393 | 1.041205 | 0.443208 | 0.196912 | 0.997568 |
| PAAD | SBS40 | Unknown | | | | | | |
| PAAD | SBS41 | Unknown | 1.119863 | 0.682336 | 1.83794 | | | |
| PAAD | SBS44 | Mismatchrepair | 2.798279 | 1.627951 | 4.809952 | 247.4016 | 0 | |
| SARC | SBS1 | Endogenous clock-like age | 0.973551 | 0.941583 | 1.006604 | 0.979032 | 0.94762 | 1.011484 |
| SARC | SBS2 | Endogenous Apobec3a | 1.006275 | 0.914311 | 1.107488 | 1.036621 | 0.918337 | 1.170139 |
| SARC | SBS3 | Homologous repair | 0.980775 | 0.937341 | 1.026221 | 0.966239 | 0.904874 | 1.031765 |
| SARC | SBS4 | Tobacco | 0.967859 | 0.912189 | 1.026925 | 1.023179 | 0.938633 | 1.115341 |
| SARC | SBS6 | Mismatchrepair | 0.990295 | 0.962486 | 1.018907 | 0.982674 | 0.94391 | 1.023029 |
| SARC | SBS7a | UV | 0.999702 | 0.994377 | 1.005056 | 1.000347 | 0.994822 | 1.005904 |
| SARC | SBS7b | UV | 0.998769 | 0.99349 | 1.004077 | 0.998673 | 0.993191 | 1.004186 |
| SARC | SBS7c | UV | 0.986183 | 0.885542 | 1.098262 | 0.952487 | 0.782476 | 1.159436 |
| SARC | SBS7d | UV | 1.052376 | 0.944387 | 1.172715 | 1.015627 | 0.873974 | 1.18024 |
| SARC | SBS8 | Unknown | 0.987133 | 0.935919 | 1.04115 | 0.995939 | 0.925583 | 1.071642 |
| SARC | SBS9 | PolETA Somatichypermutati [symbol] | 1.23103 | 0.459378 | 3.298879 | 2.21E−22 | 0 | |
| SARC | SBS10a | Polymerase epsilon hyperm [symbol] | 0.829403 | 0.609755 | 1.128175 | 0.730069 | 0.476857 | 1.117739 |
| SARC | SBS10b | Polymerase epsilon mutati [symbol] | 1.003002 | 0.981057 | 1.025437 | 0.999975 | 0.976401 | 1.024118 |
| SARC | SBS11 | Temozolo Chemotherapy | 0.881941 | 0.686403 | 1.133184 | 0.977215 | 0.826929 | 1.154815 |
| SARC | SBS12 | Liver cancer | 0.897838 | 0.766852 | 1.051197 | 0.920545 | 0.744599 | 1.138067 |
| SARC | SBS13 | Endogenous apobec | 0.999318 | 0.917641 | 1.088264 | 0.998889 | 0.902372 | 1.105729 |
| SARC | SBS14 | Mismatchrepair + PolEpsil | 0.996877 | 0.960627 | 1.034495 | 1.015868 | 0.975552 | 1.057851 |
| SARC | SBS15 | Mismatchrepair | 0.983507 | 0.953511 | 1.014446 | 0.975408 | 0.936097 | 1.01637 |
| SARC | SBS16 | Unknown | 1.028424 | 0.928973 | 1.138522 | 0.991221 | 0.862906 | 1.138618 |
| SARC | SBS17a | Unknown | 1.032515 | 0.889679 | 1.198283 | 1.063697 | 0.867025 | 1.30498 |
| SARC | SBS17b | Unknown | 1.039433 | 0.823582 | 1.311855 | 0.928609 | 0.632929 | 1.362419 |
| SARC | SBS18 | ReactiveOxy | 0.938258 | 0.849096 | 1.036782 | 0.854824 | 0.711896 | 1.026448 |
| SARC | SBS19 | Unknown | 0.974241 | 0.901617 | 1.052715 | 0.910728 | 0.816875 | 1.015365 |
| SARC | SBS21 | Mismatchrepair | 1.026451 | 0.922344 | 1.142309 | 1.040077 | 0.908358 | 1.190895 |
| SARC | SBS22 | Aristolochic acid | 1.077994 | 0.967668 | 1.200898 | 1.002737 | 0.868528 | 1.157685 |
| SARC | SBS23 | Unknown | 0.969069 | 0.853962 | 1.099692 | 0.983232 | 0.867658 | 1.114202 |
| SARC | SBS24 | Aflatoxin | 0.999878 | 0.993007 | 1.006796 | 0.998041 | 0.990604 | 1.005534 |
| SARC | SBS25 | Chemotherapy | 0.941342 | 0.791624 | 1.119377 | 0.700656 | 0.487185 | 1.007662 |
| SARC | SBS26 | Mismatchrepair | 0.978155 | 0.908214 | 1.053481 | 0.96666 | 0.870857 | 1.073002 |
| SARC | SBS28 | Unknown | 0.865281 | 0.639532 | 1.170718 | 0.587387 | 0.344842 | 1.000526 |
| SARC | SBS29 | Tobacco chewing in Hollst | 1.000152 | 0.996255 | 1.004064 | 1.000371 | 0.996413 | 1.004344 |
| SARC | SBS20 | Mismatchrepair + POLD1mut | 0.889346 | 0.76424 | 1.034932 | 0.924094 | 0.783693 | 1.089647 |
| SARC | SBS30 | BER repair-NTHL1mut | 0.980348 | 0.921733 | 1.042691 | 0.906729 | 0.818296 | 1.00472 |
| SARC | SBS31 | PLChemotherapy | 0.989043 | 0.931688 | 1.049929 | 0.980488 | 0.893957 | 1.075395 |
| SARC | SBS32 | AZA Chemotherapy | 1.006805 | 0.930258 | 1.089649 | 1.002456 | 0.901251 | 1.115025 |
| SARC | SBS33 | Unknown | 0.957552 | 0.825938 | 1.110139 | 0.908553 | 0.75044 | 1.09998 |
| SARC | SBS34 | Unknown | 1.047167 | 0.851128 | 1.288359 | 1.019586 | 0.772677 | 1.345394 |
| SARC | SBS35 | PLChemotherapy | 0.972011 | 0.898209 | 1.051878 | 0.998324 | 0.882867 | 1.12888 |
| SARC | SBS36 | BERrepair MUTY | 0.964235 | 0.827517 | 1.12354 | 1.079063 | 0.860702 | 1.352822 |
| SARC | SBS37 | Unknown | 1.055725 | 0.926602 | 1.202842 | 1.192635 | 0.990338 | 1.436256 |
| SARC | SBS38 | UV | 1.01599 | 0.908242 | 1.136519 | 0.973787 | 0.840105 | 1.128741 |
| SARC | SBS39 | Unknown | 1.006254 | 0.986264 | 1.02665 | 0.998671 | 0.975625 | 1.022261 |
| SARC | SBS42 | Haloalkane | 1.002868 | 0.936394 | 1.074061 | 0.940316 | 0.821666 | 1.076099 |
| SARC | SBS84 | Actind cytidinedeaminase | 1.0163 | 0.907792 | 1.137777 | 0.8868 | 0.747675 | 1.051812 |
| SARC | SBS85 | Actind cytidinedeaminase | 0.989763 | 0.826954 | 1.184625 | 1.126438 | 0.874323 | 1.45125 |
| SARC | SBS40 | Unknown | 2.07E−12 | 0 | | | | |
| SARC | SBS41 | Unknown | 0.878728 | 0.529646 | 1.457885 | 1.271401 | 0.263818 | 6.127176 |
| SARC | SBS44 | Mismatchrepair | 0.951682 | 0.798427 | 1.134354 | 0.803833 | 0.572206 | 1.129222 |
| SKCM | SBS1 | Endogenous clock-like age | 0.995727 | 0.988513 | 1.002994 | 0.995138 | 0.987531 | 1.002805 |
| SKCM | SBS2 | Endogenous Apobec3a | 1.007317 | 0.951673 | 1.066215 | 0.982693 | 0.90373 | 1.068556 |
| SKCM | SBS3 | Homologous repair | 1.020191 | 0.952978 | 1.092145 | 2.964196 | 0 | |
| SKCM | SBS4 | Tobacco | 1.085466 | 0.993805 | 1.185581 | 0.931244 | 0.763038 | 1.136529 |
| SKCM | SBS6 | Mismatchrepair | 0.996585 | 0.990153 | 1.003058 | 0.998257 | 0.990992 | 1.005575 |
| SKCM | SBS7a | UV | 0.999873 | 0.999532 | 1.000215 | 0.999931 | 0.999606 | 1.000257 |
| SKCM | SBS7b | UV | 0.999909 | 0.999519 | 1.000299 | 1.000004 | 0.999627 | 1.000382 |
| SKCM | SBS7c | UV | 1.002825 | 0.991115 | 1.014672 | 1.011962 | 1.000373 | 1.023684 |
| SKCM | SBS7d | UV | 1.010144 | 0.996953 | 1.023511 | 0.998622 | 0.981604 | 1.015936 |
| SKCM | SBS8 | Unknown | 1.235565 | 1.040072 | 1.467803 | 0.944396 | 0.28424 | 3.137784 |
| SKCM | SBS9 | PolETA Somatichypermutati [symbol] | 0.794081 | 0.419331 | 1.503739 | | | |
| SKCM | SBS10a | Polymerase epsilon hyperm [symbol] | 1.244704 | 0.895085 | 1.730884 | 0.823255 | 0.408301 | 1.659924 |
| SKCM | SBS10b | Polymerase epsilon mutati [symbol] | 0.999652 | 0.998395 | 1.000912 | 0.999783 | 0.998622 | 1.000945 |
| SKCM | SBS11 | Temozolo Chemotherapy | 0.981869 | 0.950241 | 1.01455 | 0.953015 | 0.903699 | 1.005022 |
| SKCM | SBS12 | Liver cancer | 1.078386 | 0.995296 | 1.168412 | 1.174069 | 1.002716 | 1.374703 |
| SKCM | SBS13 | Endogenous apobec | 1.047665 | 0.999467 | 1.098188 | 1.025301 | 0.971211 | 1.082403 |
| SKCM | SBS14 | Mismatchrepair + PolEpsil | 0.803129 | 0.474407 | 1.359626 | 1.03E+53 | 0 | |
| SKCM | SBS15 | Mismatchrepair | 0.988826 | 0.949756 | 1.029503 | 0.960557 | 0.897608 | 1.027922 |
| SKCM | SBS16 | Unknown | 1.133231 | 0.983867 | 1.30527 | 1.00431 | 0.771887 | 1.306719 |
| SKCM | SBS17a | Unknown | 0.966045 | 0.916508 | 1.018259 | 0.995243 | 0.971276 | 1.019801 |
| SKCM | SBS17b | Unknown | 0.988705 | 0.924904 | 1.056907 | 0.924369 | 0.836819 | 1.021077 |
| SKCM | SBS18 | ReactiveOxy | 1.0129 | 0.93634 | 1.09572 | 0.983224 | 0.871123 | 1.10975 |
| SKCM | SBS19 | Unknown | 0.99158 | 0.974876 | 1.008569 | 0.990005 | 0.965821 | 1.014794 |
| SKCM | SBS21 | Mismatchrepair | 0.980838 | 0.955134 | 1.007234 | 0.964277 | 0.927724 | 1.00227 |
| SKCM | SBS22 | Aristolochic acid | 1.003296 | 0.974005 | 1.033468 | 1.007038 | 0.974953 | 1.040179 |
| SKCM | SBS23 | Unknown | 0.999531 | 0.997072 | 1.001997 | 0.999078 | 0.995074 | 1.003098 |
| SKCM | SBS24 | Aflatoxin | 0.979254 | 0.912727 | 1.05063 | 0.980141 | 0.896045 | 1.072128 |
| SKCM | SBS25 | Chemotherapy | 1.666475 | 1.24918 | 2.22317 | 1309566 | 0 | |
| SKCM | SBS26 | Mismatchrepair | 0.99383 | 0.980529 | 1.007312 | 0.993613 | 0.979733 | 1.00769 |
| SKCM | SBS28 | Unknown | 0.974599 | 0.838795 | 1.132391 | 0.915381 | 0.490351 | 1.708819 |
| SKCM | SBS29 | Tobacco chewing in Hollst | 0.942453 | 0.780662 | 1.137774 | 0.753478 | 0.449907 | 1.261882 |
| SKCM | SBS20 | Mismatchrepair + POLD1mut | 0.987338 | 0.958315 | 1.01724 | 0.982958 | 0.945845 | 1.021528 |
| SKCM | SBS30 | BER repair-NTHL1mut | 1.001028 | 0.988601 | 1.013611 | 0.993016 | 0.973703 | 1.012711 |
| SKCM | SBS31 | PLChemotherapy | 0.999852 | 0.998831 | 1.000873 | 0.999616 | 0.998107 | 1.001128 |
| SKCM | SBS32 | AZA Chemotherapy | 0.99437 | 0.878381 | 1.125675 | 0.965483 | 0.766523 | 1.216086 |
| SKCM | SBS33 | Unknown | 0.980622 | 0.917976 | 1.047542 | 0.976696 | 0.898499 | 1.061699 |
| SKCM | SBS34 | Unknown | 1.332761 | 1.063535 | 1.670141 | 1.799314 | 1.073199 | 3.01671 |
| SKCM | SBS35 | PLChemotherapy | 1.004549 | 0.983047 | 1.026522 | 1.000918 | 0.972648 | 1.03001 |
| SKCM | SBS36 | BERrepair MUTY | 1.1778 | 1.002803 | 1.383335 | 1.10942 | 0.388859 | 3.165191 |
| SKCM | SBS37 | Unknown | 1.138753 | 0.965761 | 1.342733 | 1.118885 | 0.773813 | 1.617838 |
| SKCM | SBS38 | UV | 1.011944 | 0.996484 | 1.027643 | 1.010425 | 0.994397 | 1.026712 |
| SKCM | SBS39 | Unknown | 0.986111 | 0.960546 | 1.012356 | 0.986395 | 0.94962 | 1.024593 |
| SKCM | SBS42 | Haloalkane | 1.003413 | 0.987152 | 1.019941 | 1.011465 | 0.986412 | 1.037153 |
| SKCM | SBS84 | Actind cytidinedeaminase | 0.986729 | 0.930257 | 1.046629 | 0.946763 | 0.859676 | 1.042672 |
| SKCM | SBS85 | Actind cytidinedeaminase | 0.959734 | 0.661735 | 1.39193 | 1.49E−24 | 0 | |
| SKCM | SBS40 | Unknown | 2.612563 | 1.348941 | 5.059883 | | | |
| SKCM | SBS40 | Unknown | 2.612563 | 1.348941 | 5.059883 | | | |
| SKCM | SBS41 | Unknown | | | | | | |
| SKCM | SBS44 | Mismatchrepair | 1.029959 | 0.864713 | 1.226784 | 2.84E−06 | 0 | |
| STAD | SBS1 | Endogenous clock-like age | 0.998901 | 0.997146 | 1.000659 | 0.998899 | 0.997142 | 1.000659 |
| STAD | SBS2 | Endogenous Apobec3a | 0.971352 | 0.922956 | 1.022286 | 0.956455 | 0.898556 | 1.018084 |
| STAD | SBS3 | Homologous repair | 0.992333 | 0.960352 | 1.025379 | 0.892345 | 0.778376 | 1.023002 |
| STAD | SBS4 | Tobacco | 0.987137 | 0.961887 | 1.013051 | 0.971933 | 0.938014 | 1.007079 |
| STAD | SBS6 | Mismatchrepair | 0.99911 | 0.997626 | 1.000596 | 0.999331 | 0.99792 | 1.000743 |
| STAD | SBS7a | UV | 0.995297 | 0.971358 | 1.019826 | 0.973097 | 0.927417 | 1.021028 |
| STAD | SBS7b | UV | 0.970953 | 0.940827 | 1.002043 | 0.974094 | 0.937994 | 1.011584 |
| STAD | SBS7c | UV | 0.971589 | 0.88419 | 1.067627 | 0.899012 | 0.727125 | 1.111532 |
| STAD | SBS7d | UV | 1.030698 | 0.942751 | 1.126849 | 1.03295 | 0.911852 | 1.170131 |
| STAD | SBS8 | Unknown | 0.997723 | 0.979371 | 1.01642 | 0.980256 | 0.926685 | 1.036924 |
| STAD | SBS9 | PolETA Somatichypermutati [symbol] | 1.00876 | 0.947686 | 1.073769 | 0.146026 | 0.002307 | 9.241957 |
| STAD | SBS10a | Polymerase epsilon hyperm [symbol] | 0.989573 | 0.956765 | 1.023505 | 0.970762 | 0.917416 | 1.02721 |
| STAD | SBS10b | Polymerase epsilon mutati [symbol] | 0.98786 | 0.970046 | 1.006002 | 0.988042 | 0.96914 | 1.007312 |
| STAD | SBS11 | Temozolo Chemotherapy | 1.001179 | 0.974131 | 1.028978 | 0.989673 | 0.954066 | 1.02661 |
| STAD | SBS12 | Liver cancer | 1.051807 | 0.981704 | 1.126916 | 1.075033 | 0.951365 | 1.214776 |
| STAD | SBS13 | Endogenous apobec | 0.978181 | 0.938827 | 1.019183 | 0.970983 | 0.924447 | 1.019861 |
| STAD | SBS14 | Mismatchrepair + PolEpsil | 0.995019 | 0.981213 | 1.009019 | 0.997018 | 0.986775 | 1.007367 |
| STAD | SBS15 | Mismatchrepair | 0.998327 | 0.996289 | 1.00037 | 0.997682 | 0.995244 | 1.000127 |
| STAD | SBS16 | Unknown | 0.857398 | 0.734335 | 1.001083 | 0.794443 | 0.630406 | 1.001164 |
| STAD | SBS17a | Unknown | 0.998227 | 0.988111 | 1.008446 | 0.99821 | 0.987751 | 1.00878 |
| STAD | SBS17b | Unknown | 0.998204 | 0.992534 | 1.003907 | 0.998707 | 0.9931 | 1.004346 |
| STAD | SBS18 | ReactiveOxy | 0.995935 | 0.964147 | 1.02877 | 0.97967 | 0.931476 | 1.030357 |
| STAD | SBS19 | Unknown | 0.975866 | 0.909581 | 1.046982 | 0.984795 | 0.881151 | 1.10063 |
| STAD | SBS21 | Mismatchrepair | 0.993796 | 0.981541 | 1.006203 | 0.993398 | 0.980064 | 1.006913 |
| STAD | SBS22 | Aristolochic acid | 1.021431 | 0.997016 | 1.046444 | 1.014278 | 0.984263 | 1.045207 |
| STAD | SBS23 | Unknown | 0.92551 | 0.761899 | 1.124256 | 0.45891 | 0.122634 | 1.7173 |
| STAD | SBS24 | Aflatoxin | 0.992398 | 0.955156 | 1.031092 | 0.98445 | 0.934041 | 1.03758 |
| STAD | SBS25 | Chemotherapy | 0.998529 | 0.979856 | 1.017558 | 1.010175 | 0.988049 | 1.032796 |
| STAD | SBS26 | Mismatchrepair | 0.997992 | 0.99493 | 1.001063 | 0.997996 | 0.994409 | 1.001595 |
| STAD | SBS28 | Unknown | 0.994152 | 0.97748 | 1.011108 | 0.989785 | 0.968249 | 1.011799 |
| STAD | SBS29 | Tobacco chewing in Hollst | 0.975695 | 0.949437 | 1.002679 | 0.975706 | 0.943531 | 1.008977 |
| STAD | SBS20 | Mismatchrepair + POLD1mut | 1.000739 | 0.995879 | 1.005623 | 1.0026 | 0.996834 | 1.008398 |
| STAD | SBS30 | BER repair-NTHL1mut | 1.008145 | 0.976994 | 1.04029 | 1.008054 | 0.972069 | 1.045372 |
| STAD | SBS31 | PLChemotherapy | 0.984231 | 0.923245 | 1.049247 | 1.030515 | 0.927195 | 1.145349 |
| STAD | SBS32 | AZA Chemotherapy | 0.986463 | 0.907148 | 1.072713 | 0.965213 | 0.816452 | 1.141079 |
| STAD | SBS33 | Unknown | 0.986455 | 0.967408 | 1.005877 | 0.987885 | 0.966946 | 1.009276 |
| STAD | SBS34 | Unknown | 0.925416 | 0.744717 | 1.14996 | 0.978913 | 0.86828 | 1.103642 |
| STAD | SBS35 | PLChemotherapy | 0.994701 | 0.97418 | 1.015653 | 0.990998 | 0.965227 | 1.017457 |
| STAD | SBS36 | BERrepair MUTY | 1.012284 | 0.967223 | 1.059444 | 0.978849 | 0.878056 | 1.091213 |
| STAD | SBS37 | Unknown | 0.98521 | 0.939327 | 1.033335 | 0.988056 | 0.938906 | 1.03978 |
| STAD | SBS38 | UV | 0.995902 | 0.935194 | 1.060549 | 0.954404 | 0.868693 | 1.048572 |
| STAD | SBS39 | Unknown | 0.99859 | 0.98752 | 1.009783 | 0.996021 | 0.983212 | 1.008996 |
| STAD | SBS42 | Haloalkane | 0.996431 | 0.978298 | 1.014901 | 0.992876 | 0.963576 | 1.023068 |
| STAD | SBS84 | Actind cytidinedeaminase | 0.979075 | 0.846973 | 1.131781 | 0.775513 | 0.530736 | 1.133181 |
| STAD | SBS85 | Actind cytidinedeaminase | 1.087161 | 0.930772 | 1.269826 | 1.175035 | 0.921103 | 1.498971 |
| STAD | SBS40 | Unknown | 0.969256 | 0.745995 | 1.259336 | 2.01977 | 0 | |
| STAD | SBS41 | Unknown | 1.217248 | 0.631382 | 2.346744 | 2100833 | 0 | |
| STAD | SBS44 | Mismatchrepair | 1.074344 | 0.913053 | 1.264128 | 44938.89 | 0 | |
| UCEC | SBS1 | Endogenous clock-like age | 0.995467 | 0.991641 | 0.999307 | 0.995209 | 0.991248 | 0.999186 |
| UCEC | SBS2 | Endogenous Apobec3a | 0.994485 | 0.980144 | 1.009035 | 0.991268 | 0.97544 | 1.007353 |
| UCEC | SBS3 | Homologous repair | 1.014477 | 0.987944 | 1.041722 | 1.045252 | 0.96913 | 1.127353 |
| UCEC | SBS4 | Tobacco | 0.990057 | 0.968272 | 1.012333 | 0.975279 | 0.946449 | 1.004987 |
| UCEC | SBS6 | Mismatchrepair | 0.996574 | 0.993968 | 0.999187 | 0.995398 | 0.992427 | 0.998378 |
| UCEC | SBS7a | UV | 0.99096 | 0.967533 | 1.014955 | 0.985487 | 0.95348 | 1.018569 |
| UCEC | SBS7c | UV | 0.888251 | 0.746444 | 1.056999 | 0.833505 | 0.616844 | 1.126266 |
| UCEC | SBS7d | UV | 0.95721 | 0.870652 | 1.052375 | 0.858818 | 0.686921 | 1.07373 |
| UCEC | SBS8 | Unknown | 0.992802 | 0.959066 | 1.027725 | 0.948798 | 0.86254 | 1.043683 |
| UCEC | SBS9 | PolETA Somatichypermutati [symbol] | 0.55147 | 0.104898 | 2.899178 | 1.126085 | 0 | |
| UCEC | SBS10a | Polymerase epsilon hyperm [symbol] | 0.998561 | 0.996714 | 1.000411 | 0.998567 | 0.996686 | 1.000451 |
| UCEC | SBS10b | Polymerase epsilon mutati [symbol] | 0.999362 | 0.998657 | 1.000067 | 0.999342 | 0.998612 | 1.000072 |
| UCEC | SBS11 | Temozolo Chemotherapy | 0.936609 | 0.812414 | 1.079789 | 0.916964 | 0.731692 | 1.14915 |
| UCEC | SBS12 | Liver cancer | 0.993322 | 0.938705 | 1.051117 | 0.96023 | 0.856154 | 1.076958 |
| UCEC | SBS13 | Endogenous apobec | 0.999398 | 0.992411 | 1.006433 | 0.997728 | 0.98987 | 1.005649 |
| UCEC | SBS14 | Mismatchrepair + PolEpsil | 0.99853 | 0.996638 | 1.000425 | 0.998859 | 0.997174 | 1.000546 |
| UCEC | SBS15 | Mismatchrepair | 0.999791 | 0.99941 | 1.000173 | 0.999889 | 0.999518 | 1.00026 |
| UCEC | SBS16 | Unknown | 1.02735 | 0.930914 | 1.133777 | 0.887607 | 0.761484 | 1.034618 |
| UCEC | SBS17a | Unknown | 0.892296 | 0.805733 | 0.98816 | 0.896752 | 0.80032 | 1.004804 |
| UCEC | SBS17b | Unknown | 0.994908 | 0.988296 | 1.001565 | 0.99653 | 0.98972 | 1.003385 |
| UCEC | SBS18 | ReactiveOxy | 1.019476 | 0.985387 | 1.054745 | 0.994023 | 0.952547 | 1.037304 |
| UCEC | SBS19 | Unknown | 0.989805 | 0.954641 | 1.026264 | 0.988714 | 0.950701 | 1.028246 |
| UCEC | SBS21 | Mismatchrepair | 0.99977 | 0.998522 | 1.001019 | 0.999979 | 0.998994 | 1.000964 |
| UCEC | SBS22 | Aristolochic acid | 0.999413 | 0.974255 | 1.02522 | 0.99367 | 0.958971 | 1.029623 |
| UCEC | SBS23 | Unknown | 0.972301 | 0.903334 | 1.046534 | 0.936475 | 0.813924 | 1.077478 |
| UCEC | SBS24 | Aflatoxin | 0.991306 | 0.960077 | 1.02355 | 0.968558 | 0.912413 | 1.028159 |
| UCEC | SBS25 | Chemotherapy | 0.882337 | 0.634653 | 1.226683 | 1.337783 | 0 | |
| UCEC | SBS26 | Mismatchrepair | 0.998553 | 0.996334 | 1.000776 | 0.999091 | 0.997096 | 1.00109 |
| UCEC | SBS28 | Unknown | 0.992082 | 0.980096 | 1.004214 | 0.993107 | 0.981524 | 1.004827 |
| UCEC | SBS29 | Tobacco chewing in Hollst | 0.99381 | 0.972217 | 1.015883 | 0.973283 | 0.914564 | 1.035772 |
| UCEC | SBS20 | Mismatchrepair + POLD1mut | 0.991606 | 0.983384 | 0.999896 | 0.986656 | 0.975949 | 0.99748 |
| UCEC | SBS30 | BER repair-NTHL1mut | 0.996595 | 0.981031 | 1.012407 | 0.998 | 0.980323 | 1.015995 |
| UCEC | SBS31 | PLChemotherapy | 0.971295 | 0.917214 | 1.028565 | 0.962298 | 0.879176 | 1.05328 |
| UCEC | SBS32 | AZA Chemotherapy | 0.968769 | 0.913472 | 1.027413 | 0.95524 | 0.8574 | 1.064244 |
| UCEC | SBS33 | Unknown | 0.984489 | 0.971748 | 0.997397 | 0.982756 | 0.968183 | 0.997549 |
| UCEC | SBS34 | Unknown | 1.000831 | 0.915302 | 1.094352 | 0.949446 | 0.820885 | 1.098142 |
| UCEC | SBS35 | PLChemotherapy | 0.977475 | 0.886352 | 1.077967 | 0.944967 | 0.822044 | 1.08627 |
| UCEC | SBS36 | BERrepair MUTY | 0.985029 | 0.95606 | 1.014876 | 0.973866 | 0.924938 | 1.025382 |
| UCEC | SBS37 | Unknown | 0.99463 | 0.986653 | 1.002671 | 0.989674 | 0.979168 | 1.000294 |
| UCEC | SBS38 | UV | 0.878331 | 0.770314 | 1.001494 | 0.816338 | 0.667022 | 0.99908 |
| UCEC | SBS39 | Unknown | 1.009658 | 0.994202 | 1.025354 | 1.002954 | 0.984291 | 1.021971 |
| UCEC | SBS42 | Haloalkane | 0.99158 | 0.968823 | 1.014872 | 0.971814 | 0.924512 | 1.021537 |
| UCEC | SBS84 | Actind cytidinedeaminase | 0.9926 | 0.973836 | 1.011725 | 0.984446 | 0.952453 | 1.017513 |
| UCEC | SBS85 | Actind cytidinedeaminase | 0.91697 | 0.710587 | 1.183295 | 0.901636 | 0.649506 | 1.25164 |
| UCEC | SBS40 | Unknown | 0.0003 | 0 | | | | |
| UCEC | SBS41 | Unknown | 1.023469 | 0.727377 | 1.44009 | 3.5E−10 | 0 | |
| UCEC | SBS44 | Mismatchrepair | 0.990952 | 0.972496 | 1.009757 | 0.985118 | 0.957295 | 1.013751 |
| UCEC | SBS7b | UV | 0.997693 | 0.993565 | 1.001838 | 0.997015 | 0.992301 | 1.001751 |

Absence of data indicates that models did not converge, usually due to small cell sizes. [symbol]
indicates data missing or illegible when filed

TABLE 2B
Mutational signatures related to disease-specific survival (DSS), according to each cancer. Restricted to cancers with DSS events > n = 50, and binary cutpoints with cell sizes > n = 5.

| | | | Dichotomous (maxstat) signature results | | | | | | | | | | |
|---|---|---|---|---|---|---|---|---|---|---|---|---|---|
| | | | Below | | | | | | | | | | |
| | | | cut- | Below | | | | | | | | | |
| | | | point | cut- | | | | | | | | | |
| | | | no | point | | Signature | Percent | Percent | | Percent | | | |
| | | | event | event | Signature | no event | no sig | no sig | Percent | Sig no | Hazard | | |
| Cancer | Sig | Signature Label | (n) | (n) | event (n) | (n) | no event | event | sig event | event | Ratio | HRLowerCL | HRUpperCL |
| BLCA | SBS1 | Endogenous clock-like age | 203 | 103 | 20 | 69 | 0.746324 | 0.837398 | 0.162602 | 0.253676 | 0.500134 | 0.308728 | 0.810209 |
| BLCA | SBS2 | Endogenous Apobec3a | 84 | 64 | 59 | 188 | 0.308824 | 0.520325 | 0.479675 | 0.691176 | 0.447284 | 0.31223 | 0.640756 |
| BLCA | SBS4 | Tobacco | 219 | 93 | 30 | 53 | 0.805147 | 0.756098 | 0.243902 | 0.194853 | 1.111636 | 0.733305 | 1.685158 |
| BLCA | SBS6 | Mismatchrepair | 227 | 107 | 16 | 45 | 0.834559 | 0.869919 | 0.130081 | 0.165441 | 0.800584 | 0.471745 | 1.358647 |
| BLCA | SBS7a | UV | 191 | 107 | 16 | 81 | 0.702206 | 0.869919 | 0.130081 | 0.297794 | 0.355418 | 0.208379 | 0.606213 |
| BLCA | SBS7b | UV | 209 | 83 | 40 | 63 | 0.768382 | 0.674797 | 0.325203 | 0.231618 | 1.647018 | 1.122438 | 2.416764 |
| BLCA | SBS7c | UV | 237 | 102 | 21 | 35 | 0.871324 | 0.829268 | 0.170732 | 0.128676 | 1.253245 | 0.780568 | 2.012155 |
| BLCA | SBS7d | UV | 242 | 110 | 13 | 30 | 0.889706 | 0.894309 | 0.105691 | 0.110294 | 1.128211 | 0.633784 | 2.008352 |
| BLCA | SBS10a | Polymerase epsilon hyperm [symbol] | 229 | 96 | 27 | 43 | 0.841912 | 0.780488 | 0.219512 | 0.158088 | 1.682185 | 1.091556 | 2.592398 |
| BLCA | SBS10b | Polymerase epsilon mutati [symbol] | | | | | | | | | | | |
| BLCA | SBS12 | Liver cancer | 240 | 110 | 13 | 32 | 0.882353 | 0.894309 | 0.105691 | 0.117647 | 0.740546 | 0.414625 | 1.322661 |
| BLCA | SBS13 | Endogenous apobec | 115 | 75 | 48 | 157 | 0.422794 | 0.609756 | 0.390244 | 0.577206 | 0.462315 | 0.318989 | 0.67004 |
| BLCA | SBS14 | Mismatchrepair + PolEpsil | 245 | 106 | 17 | 27 | 0.900735 | 0.861789 | 0.138211 | 0.099265 | 1.233865 | 0.738066 | 2.062719 |
| BLCA | SBS15 | Mismatchrepair | 225 | 97 | 26 | 47 | 0.827206 | 0.788618 | 0.211382 | 0.172794 | 1.16743 | 0.754804 | 1.805623 |
| BLCA | SBS16 | Unknown | 235 | 114 | 9 | 37 | 0.863971 | 0.926829 | 0.073171 | 0.136029 | 0.548239 | 0.275888 | 1.089448 |
| BLCA | SBS17a | Unknown | 242 | 112 | 11 | 30 | 0.889706 | 0.910569 | 0.089431 | 0.110294 | 0.765842 | 0.411188 | 1.42639 |
| BLCA | SBS17b | Unknown | 234 | 99 | 24 | 38 | 0.860294 | 0.804878 | 0.195122 | 0.139706 | 1.545177 | 0.982165 | 2.430927 |
| BLCA | SBS18 | ReactiveOxy | 246 | 108 | 15 | 26 | 0.904412 | 0.878049 | 0.121951 | 0.095588 | 1.425972 | 0.828002 | 2.455787 |
| BLCA | SBS19 | Unknown | 237 | 112 | 11 | 35 | 0.871324 | 0.910569 | 0.089431 | 0.128676 | 0.671418 | 0.360137 | 1.251753 |
| BLCA | SBS20 | Mismatchrepair + POLD1mut | 230 | 109 | 14 | 42 | 0.845588 | 0.886179 | 0.113821 | 0.154412 | 0.681349 | 0.389441 | 1.19206 |
| BLCA | SBS21 | Mismatchrepair | 248 | 106 | 17 | 24 | 0.911765 | 0.861789 | 0.138211 | 0.088235 | 1.540263 | 0.920428 | 2.577508 |
| BLCA | SBS22 | Aristolochic acid | 227 | 116 | 7 | 45 | 0.834559 | 0.943089 | 0.056911 | 0.165441 | 0.33395 | 0.15537 | 0.717784 |
| BLCA | SBS23 | Unknown | 248 | 99 | 24 | 24 | 0.911765 | 0.804878 | 0.195122 | 0.088235 | 2.038393 | 1.301161 | 3.193338 |
| BLCA | SBS24 | Aflatoxin | 232 | 113 | 10 | 40 | 0.852941 | 0.918699 | 0.081301 | 0.147059 | 0.459174 | 0.239295 | 0.881093 |
| BLCA | SBS25 | Chemotherapy | | | | | | | | | | | |
| BLCA | SBS26 | Mismatchrepair | 213 | 104 | 19 | 59 | 0.783088 | 0.845528 | 0.154472 | 0.216912 | 0.559117 | 0.34087 | 0.917098 |
| BLCA | SBS28 | Unknown | | | | | | | | | | | |
| BLCA | SBS29 | Tobacco chewing in Hollst | 194 | 82 | 41 | 78 | 0.713235 | 0.666667 | 0.333333 | 0.286765 | 1.172878 | 0.80476 | 1.709383 |
| BLCA | SBS3 | Homologous repair | | | | | | | | | | | |
| BLCA | SBS30 | BER repair-NTHL1mut | 172 | 64 | 59 | 100 | 0.632353 | 0.520325 | 0.479675 | 0.367647 | 1.500981 | 1.051488 | 2.142624 |
| BLCA | SBS31 | PLChemotherapy | 238 | 105 | 18 | 34 | 0.875 | 0.853659 | 0.146341 | 0.125 | 1.57316 | 0.950248 | 2.604407 |
| BLCA | SBS32 | AZA Chemotherapy | 248 | 106 | 17 | 24 | 0.911765 | 0.861789 | 0.138211 | 0.088235 | 1.182944 | 0.706031 | 1.982003 |
| BLCA | SBS33 | Unknown | 166 | 65 | 58 | 106 | 0.610294 | 0.528455 | 0.471545 | 0.389706 | 1.246852 | 0.868093 | 1.790867 |
| BLCA | SBS34 | Unknown | 249 | 104 | 19 | 23 | 0.915441 | 0.845528 | 0.154472 | 0.084559 | 1.638554 | 0.998195 | 2.689713 |
| BLCA | SBS35 | PLChemotherapy | 241 | 110 | 13 | 31 | 0.886029 | 0.894309 | 0.105691 | 0.113971 | 0.812631 | 0.454475 | 1.453039 |
| BLCA | SBS36 | BERrepair MUTY | 240 | 114 | 9 | 32 | 0.882353 | 0.926829 | 0.073171 | 0.117647 | 0.520606 | 0.262945 | 1.030749 |
| BLCA | SBS38 | UV | 165 | 82 | 41 | 107 | 0.606618 | 0.666667 | 0.333333 | 0.393382 | 0.793306 | 0.544025 | 1.156813 |
| BLCA | SBS39 | Unknown | 119 | 66 | 57 | 153 | 0.4375 | 0.536585 | 0.463415 | 0.5625 | 0.607056 | 0.420714 | 0.875935 |
| BLCA | SBS42 | Haloalkane | 246 | 105 | 18 | 26 | 0.904412 | 0.853659 | 0.146341 | 0.095588 | 1.205448 | 0.729594 | 1.99166 |
| BLCA | SBS11 | Temozolo Chemotherapy | | | | | | | | | | | |
| BLCA | SBS37 | Unknown | | | | | | | | | | | |
| BLCA | SBS40 | Unknown | | | | | | | | | | | |
| BLCA | SBS41 | Unknown | | | | | | | | | | | |
| BLCA | SBS44 | Mismatchrepair | | | | | | | | | | | |
| BLCA | SBS8 | Unknown | | | | | | | | | | | |
| BLCA | SBS84 | Actind cytidinedeaminase | | | | | | | | | | | |
| BLCA | SBS85 | Actind cytidinedeaminase | | | | | | | | | | | |
| BLCA | SBS9 | PolETA Somatichypermutation | | | | | | | | | | | |
| BRCA | SBS1 | Endogenous clock-like age | 707 | 57 | 15 | 157 | 0.818287 | 0.791667 | 0.208333 | 0.181713 | 1.671837 | 0.925279 | 3.020752 |
| BRCA | SBS2 | Endogenous Apobec3a | 759 | 59 | 13 | 105 | 0.878472 | 0.819444 | 0.180556 | 0.121528 | 1.670649 | 0.911221 | 3.062995 |
| BRCA | SBS4 | Tobacco | 718 | 54 | 18 | 146 | 0.831019 | 0.75 | 0.25 | 0.168981 | 1.619767 | 0.945909 | 2.773678 |
| BRCA | SBS6 | Mismatchrepair | 697 | 50 | 22 | 167 | 0.806713 | 0.694444 | 0.305556 | 0.193287 | 1.765044 | 1.056703 | 2.948208 |
| BRCA | SBS7a | UV | 455 | 42 | 30 | 409 | 0.52662 | 0.583333 | 0.416667 | 0.47338 | 0.840224 | 0.520727 | 1.355752 |
| BRCA | SBS7b | UV | 590 | 52 | 20 | 274 | 0.68287 | 0.722222 | 0.277778 | 0.31713 | 0.760819 | 0.451156 | 1.283027 |
| BRCA | SBS7c | UV | 713 | 56 | 16 | 151 | 0.825231 | 0.777778 | 0.222222 | 0.174769 | 1.000099 | 0.570492 | 1.753219 |
| BRCA | SBS7d | UV | 632 | 48 | 24 | 232 | 0.731481 | 0.666667 | 0.333333 | 0.268519 | 1.264394 | 0.770186 | 2.07572 |
| BRCA | SBS8 | Unknown | 744 | 59 | 13 | 120 | 0.861111 | 0.819444 | 0.180556 | 0.138889 | 1.265571 | 0.690079 | 2.320995 |
| BRCA | SBS10a | Polymerase epsilon hyperm [symbol] | 652 | 50 | 22 | 212 | 0.75463 | 0.694444 | 0.305556 | 0.24537 | 1.211663 | 0.730778 | 2.008991 |
| BRCA | SBS10b | Polymerase epsilon mutati [symbol] | 768 | 56 | 16 | 96 | 0.888889 | 0.777778 | 0.222222 | 0.111111 | 2.726796 | 1.543688 | 4.816658 |
| BRCA | SBS11 | Temozolo Chemotherapy | 762 | 63 | 9 | 102 | 0.881944 | 0.875 | 0.125 | 0.118056 | 1.305714 | 0.644407 | 2.645671 |
| BRCA | SBS12 | Liver cancer | 678 | 56 | 16 | 186 | 0.784722 | 0.777778 | 0.222222 | 0.215278 | 1.100833 | 0.626255 | 1.935046 |
| BRCA | SBS13 | Endogenous apobec | 411 | 26 | 46 | 453 | 0.475694 | 0.361111 | 0.638889 | 0.524306 | 1.544854 | 0.953315 | 2.503447 |
| BRCA | SBS14 | Mismatchrepair + PolEpsil | 765 | 66 | 6 | 99 | 0.885417 | 0.916667 | 0.083333 | 0.114583 | 0.526563 | 0.225339 | 1.230448 |
| BRCA | SBS15 | Mismatchrepair | 781 | 59 | 13 | 83 | 0.903935 | 0.819444 | 0.180556 | 0.096065 | 1.483161 | 0.807085 | 2.725568 |
| BRCA | SBS16 | Unknown | 703 | 65 | 7 | 161 | 0.813657 | 0.902778 | 0.097222 | 0.186343 | 0.591673 | 0.269963 | 1.296756 |
| BRCA | SBS17a | Unknown | 547 | 42 | 30 | 317 | 0.633102 | 0.583333 | 0.416667 | 0.366898 | 1.175573 | 0.734358 | 1.881878 |
| BRCA | SBS17b | Unknown | 613 | 58 | 14 | 251 | 0.709491 | 0.805556 | 0.194444 | 0.290509 | 0.618313 | 0.343538 | 1.112864 |
| BRCA | SBS18 | ReactiveOxy | 732 | 56 | 16 | 132 | 0.847222 | 0.777778 | 0.222222 | 0.152778 | 1.668587 | 0.940123 | 2.961507 |
| BRCA | SBS19 | Unknown | 596 | 45 | 27 | 268 | 0.689815 | 0.625 | 0.375 | 0.310185 | 1.107766 | 0.685095 | 1.791204 |
| BRCA | SBS21 | Mismatchrepair | 651 | 60 | 12 | 213 | 0.753472 | 0.833333 | 0.166667 | 0.246528 | 0.722583 | 0.383007 | 1.363231 |
| BRCA | SBS22 | Aristolochic acid | 707 | 55 | 17 | 157 | 0.818287 | 0.763889 | 0.236111 | 0.181713 | 1.678556 | 0.948967 | 2.969069 |
| BRCA | SBS23 | Unknown | 772 | 66 | 6 | 92 | 0.893519 | 0.916667 | 0.083333 | 0.106481 | 0.8616 | 0.371711 | 1.997131 |
| BRCA | SBS24 | Aflatoxin | 658 | 49 | 23 | 206 | 0.761574 | 0.680556 | 0.319444 | 0.238426 | 1.745965 | 1.057609 | 2.882343 |
| BRCA | SBS25 | Chemotherapy | 835 | 69 | 3 | 29 | 0.966435 | 0.958333 | 0.041667 | 0.033565 | 1.120325 | 0.350805 | 3.577852 |
| BRCA | SBS26 | Mismatchrepair | 780 | 60 | 12 | 84 | 0.902778 | 0.833333 | 0.166667 | 0.097222 | 1.158786 | 0.598884 | 2.242144 |
| BRCA | SBS28 | Unknown | 717 | 62 | 10 | 147 | 0.829861 | 0.861111 | 0.138889 | 0.170139 | 0.832043 | 0.423866 | 1.633288 |
| BRCA | SBS29 | Tobacco chewing in Hollst | 779 | 61 | 11 | 85 | 0.90162 | 0.847222 | 0.152778 | 0.09838 | 1.435307 | 0.746296 | 2.760443 |
| BRCA | SBS20 | Mismatchrepair + POLD1mut | 779 | 57 | 15 | 85 | 0.90162 | 0.791667 | 0.208333 | 0.09838 | 3.251012 | 1.815949 | 5.82014 |
| BRCA | SBS30 | BER repair-NTHL1mut | 782 | 59 | 13 | 82 | 0.905093 | 0.819444 | 0.180556 | 0.094907 | 2.592347 | 1.404604 | 4.784456 |
| BRCA | SBS31 | PLChemotherapy | 743 | 63 | 9 | 121 | 0.859954 | 0.875 | 0.125 | 0.140046 | 0.726132 | 0.359035 | 1.468569 |
| BRCA | SBS32 | AZA Chemotherapy | 705 | 52 | 20 | 159 | 0.815972 | 0.722222 | 0.277778 | 0.184028 | 1.694012 | 1.007724 | 2.847681 |
| BRCA | SBS33 | Unknown | 697 | 55 | 17 | 167 | 0.806713 | 0.763889 | 0.236111 | 0.193287 | 1.43617 | 0.83083 | 2.482559 |
| BRCA | SBS34 | Unknown | 706 | 64 | 8 | 158 | 0.81713 | 0.888889 | 0.111111 | 0.18287 | 0.577464 | 0.275265 | 1.211434 |
| BRCA | SBS35 | PLChemotherapy | 738 | 65 | 7 | 126 | 0.854167 | 0.902778 | 0.097222 | 0.145833 | 0.750467 | 0.34274 | 1.643228 |
| BRCA | SBS36 | BERrepair MUTY | 736 | 56 | 16 | 128 | 0.851852 | 0.777778 | 0.222222 | 0.148148 | 1.709485 | 0.973319 | 3.002446 |
| BRCA | SBS37 | Unknown | 765 | 57 | 15 | 99 | 0.885417 | 0.791667 | 0.208333 | 0.114583 | 1.696127 | 0.955643 | 3.010377 |
| BRCA | SBS38 | UV | 753 | 67 | 5 | 111 | 0.871528 | 0.930556 | 0.069444 | 0.128472 | 0.577722 | 0.231411 | 1.442293 |
| BRCA | SBS39 | Unknown | 309 | 17 | 55 | 555 | 0.357639 | 0.236111 | 0.763889 | 0.642361 | 1.912283 | 1.107939 | 3.300568 |
| BRCA | SBS42 | Haloalkane | 776 | 64 | 8 | 88 | 0.898148 | 0.888889 | 0.111111 | 0.101852 | 1.297534 | 0.615474 | 2.735443 |
| BRCA | SBS84 | Actind cytidinedeaminase | 763 | 69 | 3 | 101 | 0.883102 | 0.958333 | 0.041667 | 0.116898 | 0.355439 | 0.111301 | 1.135091 |
| BRCA | SBS85 | Actind cytidinedeaminase | 766 | 61 | 11 | 98 | 0.886574 | 0.847222 | 0.152778 | 0.113426 | 1.375165 | 0.716263 | 2.640202 |
| BRCA | SBS3 | Homologous repair | | | | | | | | | | | |
| BRCA | SBS40 | Unknown | | | | | | | | | | | |
| BRCA | SBS41 | Unknown | | | | | | | | | | | |
| BRCA | SBS44 | Mismatchrepair | | | | | | | | | | | |
| BRCA | SBS9 | PolETA Somatichypermutation | | | | | | | | | | | |
| CESC | SBS1 | Endogenous clock-like age | 152 | 43 | 7 | 66 | 0.697248 | 0.86 | 0.14 | 0.302752 | 0.33667 | 0.14965 | 0.75741 |
| CESC | SBS2 | Endogenous Apobec3a | 90 | 26 | 24 | 128 | 0.412844 | 0.52 | 0.48 | 0.587156 | 0.56009 | 0.313478 | 1.000711 |
| CESC | SBS4 | Tobacco | 196 | 43 | 7 | 22 | 0.899083 | 0.86 | 0.14 | 0.100917 | 1.568587 | 0.702505 | 3.502413 |
| CESC | SBS6 | Mismatchrepair | 109 | 27 | 23 | 109 | 0.5 | 0.54 | 0.46 | 0.5 | 0.644357 | 0.359813 | 1.15392 |
| CESC | SBS7a | UV | 86 | 30 | 20 | 132 | 0.394495 | 0.6 | 0.4 | 0.605505 | 0.410394 | 0.228508 | 0.737055 |
| CESC | SBS7b | UV | 159 | 41 | 9 | 59 | 0.729358 | 0.82 | 0.18 | 0.270642 | 0.606398 | 0.293031 | 1.254882 |
| CESC | SBS7c | UV | 181 | 35 | 15 | 37 | 0.830275 | 0.7 | 0.3 | 0.169725 | 1.841501 | 1.003131 | 3.380539 |
| CESC | SBS7d | UV | 174 | 45 | 5 | 44 | 0.798165 | 0.9 | 0.1 | 0.201835 | 0.467003 | 0.183028 | 1.191577 |
| CESC | SBS8 | Unknown | 199 | 41 | 9 | 19 | 0.912844 | 0.82 | 0.18 | 0.087156 | 2.058085 | 0.98344 | 4.30704 |
| CESC | SBS10a | Polymerase epsilon hyperm [symbol] | 194 | 42 | 8 | 24 | 0.889908 | 0.84 | 0.16 | 0.110092 | 1.377072 | 0.634278 | 2.989742 |
| CESC | SBS10b | Polymerase epsilon mutati [symbol] | 51 | 18 | 32 | 167 | 0.233945 | 0.36 | 0.64 | 0.766055 | 0.534829 | 0.295725 | 0.967256 |
| CESC | SBS11 | Temozolo Chemotherapy | 202 | 49 | 1 | 16 | 0.926606 | 0.98 | 0.02 | 0.073394 | 0.324114 | 0.044589 | 2.355964 |
| CESC | SBS12 | Liver cancer | 196 | 44 | 6 | 22 | 0.899083 | 0.88 | 0.12 | 0.100917 | 1.537821 | 0.644753 | 3.667903 |
| CESC | SBS13 | Endogenous apobec | 19 | 7 | 43 | 199 | 0.087156 | 0.14 | 0.86 | 0.912844 | 0.477991 | 0.209678 | 1.089647 |
| CESC | SBS14 | Mismatchrepair + PolEpsil | 194 | 44 | 6 | 24 | 0.889908 | 0.88 | 0.12 | 0.110092 | 1.145838 | 0.485913 | 2.702013 |
| CESC | SBS15 | Mismatchrepair | 176 | 37 | 13 | 42 | 0.807339 | 0.74 | 0.26 | 0.192661 | 1.562742 | 0.810806 | 3.012021 |
| CESC | SBS16 | Unknown | 182 | 41 | 9 | 36 | 0.834862 | 0.82 | 0.18 | 0.165138 | 0.920772 | 0.443641 | 1.911056 |
| CESC | SBS17a | Unknown | 179 | 31 | 19 | 39 | 0.821101 | 0.62 | 0.38 | 0.178899 | 1.983216 | 1.096375 | 3.587411 |
| CESC | SBS17b | Unknown | 191 | 38 | 12 | 27 | 0.876147 | 0.76 | 0.24 | 0.123853 | 2.038495 | 1.057252 | 3.930437 |
| CESC | SBS18 | ReactiveOxy | 191 | 43 | 7 | 27 | 0.876147 | 0.86 | 0.14 | 0.123853 | 1.231324 | 0.549181 | 2.760764 |
| CESC | SBS19 | Unknown | 189 | 42 | 8 | 29 | 0.866972 | 0.84 | 0.16 | 0.133028 | 1.327226 | 0.618554 | 2.847816 |
| CESC | SBS21 | Mismatchrepair | 189 | 44 | 6 | 29 | 0.866972 | 0.88 | 0.12 | 0.133028 | 0.967461 | 0.410005 | 2.28285 |
| CESC | SBS22 | Aristolochic acid | 184 | 46 | 4 | 34 | 0.844037 | 0.92 | 0.08 | 0.155963 | 0.54249 | 0.193988 | 1.51708 |
| CESC | SBS23 | Unknown | 197 | 45 | 5 | 21 | 0.90367 | 0.9 | 0.1 | 0.09633 | 1.153646 | 0.45374 | 2.933176 |
| CESC | SBS24 | Aflatoxin | 196 | 41 | 9 | 22 | 0.899083 | 0.82 | 0.18 | 0.100917 | 1.565932 | 0.756319 | 3.242207 |
| CESC | SBS25 | Chemotherapy | 211 | 48 | 2 | 7 | 0.96789 | 0.96 | 0.04 | 0.03211 | 1.524658 | 0.363735 | 6.390874 |
| CESC | SBS26 | Mismatchrepair | 173 | 36 | 14 | 45 | 0.793578 | 0.72 | 0.28 | 0.206422 | 1.39325 | 0.748285 | 2.594126 |
| CESC | SBS28 | Unknown | 204 | 47 | 3 | 14 | 0.93578 | 0.94 | 0.06 | 0.06422 | 1.418833 | 0.426619 | 4.718695 |
| CESC | SBS29 | Tobacco chewing in Hollst | 192 | 47 | 3 | 26 | 0.880734 | 0.94 | 0.06 | 0.119266 | 0.563979 | 0.173992 | 1.828092 |
| CESC | SBS20 | Mismatchrepair + POLD1mut | 189 | 40 | 10 | 29 | 0.866972 | 0.8 | 0.2 | 0.133028 | 1.426297 | 0.695394 | 2.925423 |
| CESC | SBS30 | BER repair-NTHL1mut | 177 | 37 | 13 | 41 | 0.811927 | 0.74 | 0.26 | 0.188073 | 1.381189 | 0.732719 | 2.603566 |
| CESC | SBS31 | PLChemotherapy | 168 | 41 | 9 | 50 | 0.770642 | 0.82 | 0.18 | 0.229358 | 0.7782 | 0.375931 | 1.610924 |
| CESC | SBS32 | AZA Chemotherapy | 193 | 46 | 4 | 25 | 0.885321 | 0.92 | 0.08 | 0.114679 | 0.715853 | 0.256868 | 1.994974 |
| CESC | SBS33 | Unknown | 124 | 35 | 15 | 94 | 0.568807 | 0.7 | 0.3 | 0.431193 | 0.589348 | 0.320948 | 1.082206 |
| CESC | SBS34 | Unknown | 180 | 34 | 16 | 38 | 0.825688 | 0.68 | 0.32 | 0.174312 | 2.482626 | 1.352687 | 4.556438 |
| CESC | SBS35 | PLChemotherapy | 201 | 42 | 8 | 17 | 0.922018 | 0.84 | 0.16 | 0.077982 | 1.884224 | 0.875035 | 4.05732 |
| CESC | SBS36 | BERrepair MUTY | 194 | 46 | 4 | 24 | 0.889908 | 0.92 | 0.08 | 0.110092 | 0.607591 | 0.217995 | 1.693461 |
| CESC | SBS37 | Unknown | 205 | 44 | 6 | 13 | 0.940367 | 0.88 | 0.12 | 0.059633 | 2.509865 | 1.036654 | 6.076687 |
| CESC | SBS38 | UV | 169 | 32 | 18 | 49 | 0.775229 | 0.64 | 0.36 | 0.224771 | 1.905985 | 1.062727 | 3.418356 |
| CESC | SBS39 | Unknown | 69 | 19 | 31 | 149 | 0.316514 | 0.38 | 0.62 | 0.683486 | 0.590844 | 0.330015 | 1.057821 |
| CESC | SBS39 | Unknown | 69 | 19 | 31 | 149 | 0.316514 | 0.38 | 0.62 | 0.683486 | 0.590844 | 0.330015 | 1.057821 |
| CESC | SBS42 | Haloalkane | 212 | 46 | 4 | 6 | 0.972477 | 0.92 | 0.08 | 0.027523 | 2.066647 | 0.73589 | 5.803897 |
| CESC | SBS84 | Actind cytidinedeaminase | 196 | 45 | 5 | 22 | 0.899083 | 0.9 | 0.1 | 0.100917 | 0.93845 | 0.367073 | 2.399217 |
| CESC | SBS85 | Actind cytidinedeaminase | 200 | 46 | 4 | 18 | 0.917431 | 0.92 | 0.08 | 0.082569 | 1.128717 | 0.401412 | 3.173797 |
| CESC | SBS3 | Homologous repair | | | | | | | | | | | |
| CESC | SBS40 | Unknown | | | | | | | | | | | |
| CESC | SBS41 | Unknown | | | | | | | | | | | |
| CESC | SBS44 | Mismatchrepair | | | | | | | | | | | |
| CESC | SBS9 | PolETA Somatichypermutation | | | | | | | | | | | |
| COAD | SBS1 | Endogenous clock-like age | 158 | 36 | 20 | 148 | 0.51634 | 0.642857 | 0.357143 | 0.48366 | 0.683255 | 0.385238 | 1.211815 |
| COAD | SBS2 | Endogenous Apobec3a | 272 | 46 | 10 | 34 | 0.888889 | 0.821429 | 0.178571 | 0.111111 | 1.53501 | 0.757508 | 3.110536 |
| COAD | SBS4 | Tobacco | 236 | 50 | 6 | 70 | 0.771242 | 0.892857 | 0.107143 | 0.228758 | 0.42192 | 0.179614 | 0.991107 |
| COAD | SBS6 | Mismatchrepair | 210 | 43 | 13 | 96 | 0.686275 | 0.767857 | 0.232143 | 0.313725 | 0.918573 | 0.488485 | 1.727332 |
| COAD | SBS7a | UV | 269 | 52 | 4 | 37 | 0.879085 | 0.928571 | 0.071429 | 0.120915 | 0.521596 | 0.18786 | 1.448217 |
| COAD | SBS7b | UV | 294 | 56 | 0 | 12 | 0.960784 | 1 | 0 | 0.039216 | 8.92E−07 | 0 | |
| COAD | SBS7c | UV | 267 | 55 | 1 | 39 | 0.872549 | 0.982143 | 0.017857 | 0.127451 | 0.13014 | 0.017965 | 0.94273 |
| COAD | SBS7d | UV | 235 | 38 | 18 | 71 | 0.767974 | 0.678571 | 0.321429 | 0.232026 | 1.181115 | 0.668342 | 2.087306 |
| COAD | SBS8 | Unknown | 275 | 49 | 7 | 31 | 0.898693 | 0.875 | 0.125 | 0.101307 | 1.458696 | 0.655088 | 3.248103 |
| COAD | SBS10a | Polymerase epsilon hyperm [symbol] | 269 | 46 | 10 | 37 | 0.879085 | 0.821429 | 0.178571 | 0.120915 | 2.417657 | 1.187557 | 4.921923 |
| COAD | SBS10b | Polymerase epsilon mutati [symbol] | 221 | 34 | 22 | 85 | 0.722222 | 0.607143 | 0.392857 | 0.277778 | 1.772984 | 1.030194 | 3.051339 |
| COAD | SBS11 | Temozolo Chemotherapy | 288 | 52 | 4 | 18 | 0.941176 | 0.928571 | 0.071429 | 0.058824 | 1.588154 | 0.548924 | 4.594866 |
| COAD | SBS12 | Liver cancer | 244 | 49 | 7 | 62 | 0.797386 | 0.875 | 0.125 | 0.202614 | 0.811695 | 0.361912 | 1.820468 |
| COAD | SBS13 | Endogenous apobec | 155 | 33 | 23 | 151 | 0.506536 | 0.589286 | 0.410714 | 0.493464 | 0.7579 | 0.443277 | 1.295829 |
| COAD | SBS14 | Mismatchrepair + PolEpsil | 253 | 49 | 7 | 53 | 0.826797 | 0.875 | 0.125 | 0.173203 | 0.962922 | 0.430681 | 2.152917 |
| COAD | SBS15 | Mismatchrepair | 260 | 49 | 7 | 46 | 0.849673 | 0.875 | 0.125 | 0.150327 | 1.198488 | 0.529964 | 2.710323 |
| COAD | SBS16 | Unknown | 241 | 48 | 8 | 65 | 0.787582 | 0.857143 | 0.142857 | 0.212418 | 0.830431 | 0.389705 | 1.769584 |
| COAD | SBS17a | Unknown | 145 | 19 | 37 | 161 | 0.473856 | 0.339286 | 0.660714 | 0.526144 | 1.537468 | 0.878091 | 2.691984 |
| COAD | SBS17b | Unknown | 277 | 45 | 11 | 29 | 0.905229 | 0.803571 | 0.196429 | 0.094771 | 3.124651 | 1.567689 | 6.227922 |
| COAD | SBS18 | ReactiveOxy | 271 | 45 | 11 | 35 | 0.885621 | 0.803571 | 0.196429 | 0.114379 | 1.838655 | 0.946737 | 3.570846 |
| COAD | SBS19 | Unknown | 275 | 49 | 7 | 31 | 0.898693 | 0.875 | 0.125 | 0.101307 | 1.383912 | 0.622125 | 3.078505 |
| COAD | SBS21 | Mismatchrepair | 274 | 46 | 10 | 32 | 0.895425 | 0.821429 | 0.178571 | 0.104575 | 2.401711 | 1.191516 | 4.841074 |
| COAD | SBS22 | Aristolochic acid | 279 | 45 | 11 | 27 | 0.911765 | 0.803571 | 0.196429 | 0.088235 | 1.815568 | 0.908773 | 3.627184 |
| COAD | SBS23 | Unknown | 283 | 50 | 6 | 23 | 0.924837 | 0.892857 | 0.107143 | 0.075163 | 1.156822 | 0.494046 | 2.708728 |
| COAD | SBS24 | Aflatoxin | 239 | 46 | 10 | 67 | 0.781046 | 0.821429 | 0.178571 | 0.218954 | 0.796819 | 0.396088 | 1.602976 |
| COAD | SBS25 | Chemotherapy | 289 | 44 | 12 | 17 | 0.944444 | 0.785714 | 0.214286 | 0.055556 | 4.379459 | 2.261667 | 8.480319 |
| COAD | SBS26 | Mismatchrepair | 210 | 27 | 29 | 96 | 0.686275 | 0.482143 | 0.517857 | 0.313725 | 2.832078 | 1.663024 | 4.822939 |
| COAD | SBS28 | Unknown | 264 | 47 | 9 | 42 | 0.862745 | 0.839286 | 0.160714 | 0.137255 | 0.913583 | 0.441247 | 1.891536 |
| COAD | SBS29 | Tobacco chewing in Hollst | 259 | 52 | 4 | 47 | 0.846405 | 0.928571 | 0.071429 | 0.153595 | 0.370419 | 0.130157 | 1.054189 |
| COAD | SBS20 | Mismatchrepair + POLD1mut | 235 | 46 | 10 | 71 | 0.767974 | 0.821429 | 0.178571 | 0.232026 | 1.024278 | 0.509805 | 2.057933 |
| COAD | SBS30 | BER repair-NTHL1mut | 270 | 42 | 14 | 36 | 0.882353 | 0.75 | 0.25 | 0.117647 | 2.065213 | 1.118217 | 3.8142 |
| COAD | SBS31 | PLChemotherapy | 250 | 49 | 7 | 56 | 0.816993 | 0.875 | 0.125 | 0.183007 | 0.75238 | 0.338561 | 1.672006 |
| COAD | SBS32 | AZA Chemotherapy | 258 | 50 | 6 | 48 | 0.843137 | 0.892857 | 0.107143 | 0.156863 | 0.638759 | 0.272242 | 1.498716 |
| COAD | SBS33 | Unknown | 218 | 35 | 21 | 88 | 0.712418 | 0.625 | 0.375 | 0.287582 | 2.005136 | 1.157394 | 3.473815 |
| COAD | SBS34 | Unknown | 239 | 46 | 10 | 67 | 0.781046 | 0.821429 | 0.178571 | 0.218954 | 0.882891 | 0.442378 | 1.762057 |
| COAD | SBS35 | PLChemotherapy | 241 | 46 | 10 | 65 | 0.787582 | 0.821429 | 0.178571 | 0.212418 | 0.916887 | 0.459244 | 1.830578 |
| COAD | SBS36 | BERrepair MUTY | 270 | 48 | 8 | 36 | 0.882353 | 0.857143 | 0.142857 | 0.117647 | 1.082904 | 0.502646 | 2.333018 |
| COAD | SBS37 | Unknown | 276 | 48 | 8 | 30 | 0.901961 | 0.857143 | 0.142857 | 0.098039 | 1.572165 | 0.731335 | 3.379712 |
| COAD | SBS38 | UV | 260 | 43 | 13 | 46 | 0.849673 | 0.767857 | 0.232143 | 0.150327 | 1.392243 | 0.745083 | 2.601511 |
| COAD | SBS39 | Unknown | 275 | 44 | 12 | 31 | 0.898693 | 0.785714 | 0.214286 | 0.101307 | 1.888321 | 0.981806 | 3.631835 |
| COAD | SBS42 | Haloalkane | 287 | 49 | 7 | 19 | 0.937908 | 0.875 | 0.125 | 0.062092 | 2.219681 | 0.97549 | 5.05078 |
| COAD | SBS84 | Actind cytidinedeaminase | 278 | 50 | 6 | 28 | 0.908497 | 0.892857 | 0.107143 | 0.091503 | 1.057629 | 0.449534 | 2.488307 |
| COAD | SBS85 | Actind cytidinedeaminase | 282 | 53 | 3 | 24 | 0.921569 | 0.946429 | 0.053571 | 0.078431 | 0.666061 | 0.206213 | 2.151354 |
| COAD | SBS3 | Homologous repair | | | | | | | | | | | |
| COAD | SBS40 | Unknown | | | | | | | | | | | |
| COAD | SBS41 | Unknown | | | | | | | | | | | |
| COAD | SBS44 | Mismatchrepair | | | | | | | | | | | |
| COAD | SBS9 | PolETA Somatichypermutation | | | | | | | | | | | |
| HNSC | SBS1 | Endogenous clock-like age | 69 | 35 | 94 | 281 | 0.197143 | 0.271318 | 0.728682 | 0.802857 | 0.724196 | 0.487196 | 1.076486 |
| HNSC | SBS2 | Endogenous Apobec3a | 89 | 20 | 109 | 261 | 0.254286 | 0.155039 | 0.844961 | 0.745714 | 1.463775 | 0.902592 | 2.373871 |
| HNSC | SBS4 | Tobacco | 304 | 115 | 14 | 46 | 0.868571 | 0.891473 | 0.108527 | 0.131429 | 0.730471 | 0.416722 | 1.280441 |
| HNSC | SBS6 | Mismatchrepair | 234 | 89 | 40 | 116 | 0.668571 | 0.689922 | 0.310078 | 0.331429 | 0.80349 | 0.550644 | 1.172439 |
| HNSC | SBS7a | UV | 187 | 61 | 68 | 163 | 0.534286 | 0.472868 | 0.527132 | 0.465714 | 1.3379 | 0.943421 | 1.897325 |
| HNSC | SBS7a | UV | 187 | 61 | 68 | 163 | 0.534286 | 0.472868 | 0.527132 | 0.465714 | 1.3379 | 0.943421 | 1.897325 |
| HNSC | SBS7b | UV | 227 | 88 | 41 | 123 | 0.648571 | 0.682171 | 0.317829 | 0.351429 | 0.728851 | 0.500165 | 1.062097 |
| HNSC | SBS7c | UV | 300 | 99 | 30 | 50 | 0.857143 | 0.767442 | 0.232558 | 0.142857 | 1.519512 | 1.003616 | 2.300596 |
| HNSC | SBS7d | UV | 269 | 95 | 34 | 81 | 0.768571 | 0.736434 | 0.263566 | 0.231429 | 1.162953 | 0.783371 | 1.726461 |
| HNSC | SBS8 | Unknown | 311 | 109 | 20 | 39 | 0.888571 | 0.844961 | 0.155039 | 0.111429 | 1.411243 | 0.874135 | 2.278374 |
| HNSC | SBS10a | Polymerase epsilon hyperm [symbol] | 316 | 114 | 15 | 34 | 0.902857 | 0.883721 | 0.116279 | 0.097143 | 1.312249 | 0.762574 | 2.258138 |
| HNSC | SBS10b | Polymerase epsilon mutati [symbol] | 75 | 39 | 90 | 275 | 0.214286 | 0.302326 | 0.697674 | 0.785714 | 0.694424 | 0.473708 | 1.01798 |
| HNSC | SBS11 | Temozolo Chemotherapy | 309 | 121 | 8 | 41 | 0.882857 | 0.937984 | 0.062016 | 0.117143 | 0.592884 | 0.289324 | 1.214942 |
| HNSC | SBS12 | Liver cancer | 313 | 117 | 12 | 37 | 0.894286 | 0.906977 | 0.093023 | 0.105714 | 0.823754 | 0.453156 | 1.497434 |
| HNSC | SBS13 | Endogenous apobec | 143 | 37 | 92 | 207 | 0.408571 | 0.286822 | 0.713178 | 0.591429 | 1.233165 | 0.830552 | 1.830947 |
| HNSC | SBS14 | Mismatchrepair + PolEpsil | 317 | 113 | 16 | 33 | 0.905714 | 0.875969 | 0.124031 | 0.094286 | 1.090116 | 0.630925 | 1.883508 |
| HNSC | SBS15 | Mismatchrepair | 239 | 96 | 33 | 111 | 0.682857 | 0.744186 | 0.255814 | 0.317143 | 0.726038 | 0.487862 | 1.080492 |
| HNSC | SBS16 | Unknown | 238 | 74 | 55 | 112 | 0.68 | 0.573643 | 0.426357 | 0.32 | 1.489074 | 1.043252 | 2.125413 |
| HNSC | SBS17a | Unknown | 313 | 110 | 19 | 37 | 0.894286 | 0.852713 | 0.147287 | 0.105714 | 1.232297 | 0.754167 | 2.013552 |
| HNSC | SBS17b | Unknown | 285 | 104 | 25 | 65 | 0.814286 | 0.806202 | 0.193798 | 0.185714 | 0.880317 | 0.563962 | 1.374133 |
| HNSC | SBS18 | ReactiveOxy | 278 | 111 | 18 | 72 | 0.794286 | 0.860465 | 0.139535 | 0.205714 | 0.711059 | 0.427079 | 1.183868 |
| HNSC | SBS19 | Unknown | 242 | 99 | 30 | 108 | 0.691429 | 0.767442 | 0.232558 | 0.308571 | 0.688823 | 0.456755 | 1.0388 |
| HNSC | SBS21 | Mismatchrepair | 291 | 99 | 30 | 59 | 0.831429 | 0.767442 | 0.232558 | 0.168571 | 1.303049 | 0.859598 | 1.975267 |
| HNSC | SBS22 | Aristolochic acid | 194 | 83 | 46 | 156 | 0.554286 | 0.643411 | 0.356589 | 0.445714 | 0.681967 | 0.473663 | 0.981877 |
| HNSC | SBS23 | Unknown | 309 | 109 | 20 | 41 | 0.882857 | 0.844961 | 0.155039 | 0.117143 | 1.354365 | 0.837198 | 2.191006 |
| HNSC | SBS24 | Aflatoxin | 244 | 77 | 52 | 106 | 0.697143 | 0.596899 | 0.403101 | 0.302857 | 1.290572 | 0.904553 | 1.841326 |
| HNSC | SBS25 | Chemotherapy | 316 | 107 | 22 | 34 | 0.902857 | 0.829457 | 0.170543 | 0.097143 | 1.477547 | 0.927779 | 2.353088 |
| HNSC | SBS26 | Mismatchrepair | 240 | 80 | 49 | 110 | 0.685714 | 0.620155 | 0.379845 | 0.314286 | 1.218511 | 0.851351 | 1.744015 |
| HNSC | SBS28 | Unknown | 310 | 119 | 10 | 40 | 0.885714 | 0.922481 | 0.077519 | 0.114286 | 0.877571 | 0.456841 | 1.685776 |
| HNSC | SBS29 | Tobacco chewing in Hollst | 302 | 96 | 33 | 48 | 0.862857 | 0.744186 | 0.255814 | 0.137143 | 1.579499 | 1.058431 | 2.357091 |
| HNSC | SBS20 | Mismatchrepair + POLD1mut | 295 | 117 | 12 | 55 | 0.842857 | 0.906977 | 0.093023 | 0.157143 | 0.630618 | 0.347427 | 1.144638 |
| HNSC | SBS30 | BER repair-NTHL1mut | 261 | 88 | 41 | 89 | 0.745714 | 0.682171 | 0.317829 | 0.254286 | 1.146513 | 0.788941 | 1.666148 |
| HNSC | SBS31 | PLChemotherapy | 318 | 107 | 22 | 32 | 0.908571 | 0.829457 | 0.170543 | 0.091429 | 1.606574 | 1.005138 | 2.567886 |
| HNSC | SBS32 | AZA Chemotherapy | 318 | 112 | 17 | 32 | 0.908571 | 0.868217 | 0.131783 | 0.091429 | 1.276771 | 0.764324 | 2.13279 |
| HNSC | SBS33 | Unknown | 299 | 103 | 26 | 51 | 0.854286 | 0.79845 | 0.20155 | 0.145714 | 1.181745 | 0.763197 | 1.829833 |
| HNSC | SBS34 | Unknown | 312 | 110 | 19 | 38 | 0.891429 | 0.852713 | 0.147287 | 0.108571 | 1.515788 | 0.919947 | 2.497552 |
| HNSC | SBS35 | PLChemotherapy | 310 | 106 | 23 | 40 | 0.885714 | 0.821705 | 0.178295 | 0.114286 | 1.663212 | 1.050589 | 2.63307 |
| HNSC | SBS36 | BERrepair MUTY | 319 | 111 | 18 | 31 | 0.911429 | 0.860465 | 0.139535 | 0.088571 | 1.659203 | 1.000755 | 2.750876 |
| HNSC | SBS38 | UV | 308 | 105 | 24 | 42 | 0.88 | 0.813953 | 0.186047 | 0.12 | 1.34324 | 0.859231 | 2.099894 |
| HNSC | SBS39 | Unknown | 280 | 98 | 31 | 70 | 0.8 | 0.75969 | 0.24031 | 0.2 | 1.120706 | 0.746561 | 1.682359 |
| HNSC | SBS42 | Haloalkane | 311 | 114 | 15 | 39 | 0.888571 | 0.883721 | 0.116279 | 0.111429 | 1.232683 | 0.716849 | 2.119706 |
| HNSC | SBS84 | Actind cytidinedeaminase | 319 | 112 | 17 | 31 | 0.911429 | 0.868217 | 0.131783 | 0.088571 | 1.651075 | 0.973713 | 2.799643 |
| HNSC | SBS3 | Homologous repair | | | | | | | | | | | |
| HNSC | SBS37 | Unknown | | | | | | | | | | | |
| HNSC | SBS40 | Unknown | | | | | | | | | | | |
| HNSC | SBS41 | Unknown | | | | | | | | | | | |
| HNSC | SBS44 | Mismatchrepair | | | | | | | | | | | |
| HNSC | SBS85 | Actind cytidinedeaminase | | | | | | | | | | | |
| HNSC | SBS9 | PolETA Somatichypermutation | | | | | | | | | | | |
| LGG | SBS1 | Endogenous clock-like age | 337 | 76 | 35 | 39 | 0.896277 | 0.684685 | 0.315315 | 0.103723 | 3.654361 | 2.226141 | 5.998882 |
| LGG | SBS2 | Endogenous Apobec3a | 298 | 82 | 29 | 78 | 0.792553 | 0.738739 | 0.261261 | 0.207447 | 0.934106 | 0.599564 | 1.455313 |
| LGG | SBS4 | Tobacco | 343 | 95 | 16 | 33 | 0.912234 | 0.855856 | 0.144144 | 0.087766 | 1.575856 | 0.915882 | 2.711397 |
| LGG | SBS6 | Mismatchrepair | 340 | 93 | 18 | 36 | 0.904255 | 0.837838 | 0.162162 | 0.095745 | 1.180319 | 0.701016 | 1.987333 |
| LGG | SBS7a | UV | 326 | 92 | 19 | 50 | 0.867021 | 0.828829 | 0.171171 | 0.132979 | 1.22653 | 0.737834 | 2.038908 |
| LGG | SBS7b | UV | 259 | 82 | 29 | 117 | 0.68883 | 0.738739 | 0.261261 | 0.31117 | 0.780855 | 0.506413 | 1.204025 |
| LGG | SBS7c | UV | 333 | 100 | 11 | 43 | 0.885638 | 0.900901 | 0.099099 | 0.114362 | 0.699547 | 0.371036 | 1.318918 |
| LGG | SBS7d | UV | 309 | 90 | 21 | 67 | 0.821809 | 0.810811 | 0.189189 | 0.178191 | 0.967204 | 0.596647 | 1.567901 |
| LGG | SBS8 | Unknown | 338 | 89 | 22 | 38 | 0.898936 | 0.801802 | 0.198198 | 0.101064 | 2.25033 | 1.395927 | 3.627687 |
| LGG | SBS10a | Polymerase epsilon hyperm [symbol] | 311 | 87 | 24 | 65 | 0.827128 | 0.783784 | 0.216216 | 0.172872 | 0.910572 | 0.569912 | 1.454857 |
| LGG | SBS10b | Polymerase epsilon mutati [symbol] | 331 | 90 | 21 | 45 | 0.880319 | 0.810811 | 0.189189 | 0.119681 | 1.600953 | 0.978485 | 2.619406 |
| LGG | SBS11 | Temozolo Chemotherapy | 340 | 93 | 18 | 36 | 0.904255 | 0.837838 | 0.162162 | 0.095745 | 1.267021 | 0.757543 | 2.119143 |
| LGG | SBS12 | Liver cancer | 324 | 103 | 8 | 52 | 0.861702 | 0.927928 | 0.072072 | 0.138298 | 0.302621 | 0.144395 | 0.634227 |
| LGG | SBS13 | Endogenous apobec | 210 | 60 | 51 | 166 | 0.558511 | 0.540541 | 0.459459 | 0.441489 | 1.29013 | 0.876994 | 1.897889 |
| LGG | SBS14 | Mismatchrepair + PolEpsil | 333 | 101 | 10 | 43 | 0.885638 | 0.90991 | 0.09009 | 0.114362 | 1.040545 | 0.539013 | 2.008735 |
| LGG | SBS15 | Mismatchrepair | 326 | 93 | 18 | 50 | 0.867021 | 0.837838 | 0.162162 | 0.132979 | 1.089912 | 0.637901 | 1.862214 |
| LGG | SBS16 | Unknown | 261 | 79 | 32 | 115 | 0.694149 | 0.711712 | 0.288288 | 0.305851 | 0.683213 | 0.444678 | 1.049703 |
| LGG | SBS17a | Unknown | 324 | 96 | 15 | 52 | 0.861702 | 0.864865 | 0.135135 | 0.138298 | 1.385515 | 0.7889 | 2.433325 |
| LGG | SBS17b | Unknown | 339 | 93 | 18 | 37 | 0.901596 | 0.837838 | 0.162162 | 0.098404 | 1.143015 | 0.675108 | 1.93522 |
| LGG | SBS18 | ReactiveOxy | 371 | 109 | 2 | 5 | 0.986702 | 0.981982 | 0.018018 | 0.013298 | 2.386319 | 0.574945 | 9.904458 |
| LGG | SBS19 | Unknown | 345 | 92 | 19 | 31 | 0.917553 | 0.828829 | 0.171171 | 0.082447 | 0.935787 | 0.551879 | 1.586755 |
| LGG | SBS21 | Mismatchrepair | 339 | 98 | 13 | 37 | 0.901596 | 0.882883 | 0.117117 | 0.098404 | 0.533238 | 0.291483 | 0.975503 |
| LGG | SBS22 | Aristolochic acid | 342 | 95 | 16 | 34 | 0.909574 | 0.855856 | 0.144144 | 0.090426 | 1.194701 | 0.680095 | 2.098691 |
| LGG | SBS23 | Unknown | 296 | 91 | 20 | 80 | 0.787234 | 0.81982 | 0.18018 | 0.212766 | 0.603005 | 0.364159 | 0.998507 |
| LGG | SBS24 | Aflatoxin | 318 | 90 | 21 | 58 | 0.845745 | 0.810811 | 0.189189 | 0.154255 | 1.136254 | 0.696567 | 1.853482 |
| LGG | SBS25 | Chemotherapy | 370 | 106 | 5 | 6 | 0.984043 | 0.954955 | 0.045045 | 0.015957 | 7.7913 | 3.043813 | 19.94352 |
| LGG | SBS26 | Mismatchrepair | 264 | 87 | 24 | 112 | 0.702128 | 0.783784 | 0.216216 | 0.297872 | 0.792357 | 0.49663 | 1.264179 |
| LGG | SBS28 | Unknown | 338 | 95 | 16 | 38 | 0.898936 | 0.855856 | 0.144144 | 0.101064 | 1.140422 | 0.66374 | 1.959446 |
| LGG | SBS29 | Tobacco chewing in Hollst | 321 | 91 | 20 | 55 | 0.853723 | 0.81982 | 0.18018 | 0.146277 | 1.220996 | 0.746381 | 1.997413 |
| LGG | SBS20 | Mismatchrepair + POLD1mut | 338 | 99 | 12 | 38 | 0.898936 | 0.891892 | 0.108108 | 0.101064 | 1.371327 | 0.739201 | 2.544014 |
| LGG | SBS30 | BER repair-NTHL1mut | 338 | 96 | 15 | 38 | 0.898936 | 0.864865 | 0.135135 | 0.101064 | 1.743756 | 0.98859 | 3.075781 |
| LGG | SBS31 | PLChemotherapy | 320 | 86 | 25 | 56 | 0.851064 | 0.774775 | 0.225225 | 0.148936 | 1.697361 | 1.066546 | 2.701276 |
| LGG | SBS32 | AZA Chemotherapy | 337 | 96 | 15 | 39 | 0.896277 | 0.864865 | 0.135135 | 0.103723 | 1.268465 | 0.723936 | 2.222575 |
| LGG | SBS33 | Unknown | 342 | 94 | 17 | 34 | 0.909574 | 0.846847 | 0.153153 | 0.090426 | 1.690807 | 0.998442 | 2.863289 |
| LGG | SBS34 | Unknown | 329 | 100 | 11 | 47 | 0.875 | 0.900901 | 0.099099 | 0.125 | 1.094364 | 0.57817 | 2.071421 |
| LGG | SBS35 | PLChemotherapy | 323 | 101 | 10 | 53 | 0.859043 | 0.90991 | 0.09009 | 0.140957 | 0.93842 | 0.481539 | 1.828785 |
| LGG | SBS36 | BERrepair MUTY | 368 | 108 | 3 | 8 | 0.978723 | 0.972973 | 0.027027 | 0.021277 | 1.016654 | 0.320422 | 3.225703 |
| LGG | SBS37 | Unknown | 304 | 93 | 18 | 72 | 0.808511 | 0.837838 | 0.162162 | 0.191489 | 0.990987 | 0.589704 | 1.665334 |
| LGG | SBS38 | UV | 336 | 98 | 13 | 40 | 0.893617 | 0.882883 | 0.117117 | 0.106383 | 0.870465 | 0.485337 | 1.561202 |
| LGG | SBS39 | Unknown | 269 | 66 | 45 | 107 | 0.715426 | 0.594595 | 0.405405 | 0.284574 | 1.260789 | 0.852748 | 1.864078 |
| LGG | SBS42 | Haloalkane | 308 | 95 | 16 | 68 | 0.819149 | 0.855856 | 0.144144 | 0.180851 | 0.704425 | 0.403211 | 1.230657 |
| LGG | SBS84 | Actind cytidinedeaminase | 306 | 93 | 18 | 70 | 0.81383 | 0.837838 | 0.162162 | 0.18617 | 0.869971 | 0.516372 | 1.465707 |
| LGG | SBS85 | Actind cytidinedeaminase | 321 | 104 | 7 | 55 | 0.853723 | 0.936937 | 0.063063 | 0.146277 | 0.727195 | 0.333993 | 1.583307 |
| LGG | SBS3 | Homologous repair | | | | | | | | | | | |
| LGG | SBS40 | Unknown | | | | | | | | | | | |
| LGG | SBS41 | Unknown | | | | | | | | | | | |
| LGG | SBS44 | Mismatchrepair | | | | | | | | | | | |
| LGG | SBS9 | PolETA Somatichypermutation | | | | | | | | | | | |
| LIHC | SBS1 | Endogenous clock-like age | 142 | 24 | 51 | 127 | 0.527881 | 0.32 | 0.68 | 0.472119 | 2.307518 | 1.400122 | 3.802983 |
| LIHC | SBS2 | Endogenous Apobec3a | 228 | 60 | 15 | 41 | 0.847584 | 0.8 | 0.2 | 0.152416 | 1.410724 | 0.783835 | 2.538982 |
| LIHC | SBS3 | Homologous repair | 232 | 67 | 8 | 37 | 0.862454 | 0.893333 | 0.106667 | 0.137546 | 0.792611 | 0.367915 | 1.707551 |
| LIHC | SBS4 | Tobacco | 207 | 51 | 24 | 62 | 0.769517 | 0.68 | 0.32 | 0.230483 | 2.039176 | 1.22491 | 3.394732 |
| LIHC | SBS6 | Mismatchrepair | 117 | 46 | 29 | 152 | 0.434944 | 0.613333 | 0.386667 | 0.565056 | 0.516619 | 0.317067 | 0.841764 |
| LIHC | SBS7a | UV | 230 | 59 | 16 | 39 | 0.855019 | 0.786667 | 0.213333 | 0.144981 | 2.177722 | 1.228743 | 3.859613 |
| LIHC | SBS7b | UV | 230 | 66 | 9 | 39 | 0.855019 | 0.88 | 0.12 | 0.144981 | 0.704507 | 0.342765 | 1.448018 |
| LIHC | SBS7d | UV | 216 | 61 | 14 | 53 | 0.802974 | 0.813333 | 0.186667 | 0.197026 | 0.870813 | 0.47848 | 1.584845 |
| LIHC | SBS8 | Unknown | 228 | 65 | 10 | 41 | 0.847584 | 0.866667 | 0.133333 | 0.152416 | 0.75899 | 0.378647 | 1.521379 |
| LIHC | SBS10a | Polymerase epsilon hyperm [symbol] | 164 | 56 | 19 | 105 | 0.609665 | 0.746667 | 0.253333 | 0.390335 | 0.669012 | 0.396031 | 1.130158 |
| LIHC | SBS10b | Polymerase epsilon mutati [symbol] | 169 | 48 | 27 | 100 | 0.628253 | 0.64 | 0.36 | 0.371747 | 0.92262 | 0.570552 | 1.491937 |
| LIHC | SBS11 | Temozolo Chemotherapy | 239 | 67 | 8 | 30 | 0.888476 | 0.893333 | 0.106667 | 0.111524 | 1.413619 | 0.668811 | 2.987868 |
| LIHC | SBS12 | Liver cancer | 243 | 64 | 11 | 26 | 0.903346 | 0.853333 | 0.146667 | 0.096654 | 1.64297 | 0.852176 | 3.167594 |
| LIHC | SBS13 | Endogenous apobec | 244 | 63 | 12 | 25 | 0.907063 | 0.84 | 0.16 | 0.092937 | 1.064942 | 0.5535 | 2.048964 |
| LIHC | SBS14 | Mismatchrepair + PolEpsil | 241 | 65 | 10 | 28 | 0.895911 | 0.866667 | 0.133333 | 0.104089 | 1.338222 | 0.678796 | 2.638257 |
| LIHC | SBS15 | Mismatchrepair | 225 | 60 | 15 | 44 | 0.836431 | 0.8 | 0.2 | 0.163569 | 1.689097 | 0.943703 | 3.023246 |
| LIHC | SBS16 | Unknown | 120 | 45 | 30 | 149 | 0.446097 | 0.6 | 0.4 | 0.553903 | 0.701739 | 0.433954 | 1.13477 |
| LIHC | SBS17a | Unknown | 127 | 29 | 46 | 142 | 0.472119 | 0.386667 | 0.613333 | 0.527881 | 1.371471 | 0.84631 | 2.22251 |
| LIHC | SBS17b | Unknown | 242 | 66 | 9 | 27 | 0.899628 | 0.88 | 0.12 | 0.100372 | 1.755056 | 0.847447 | 3.63471 |
| LIHC | SBS18 | ReactiveOxy | 244 | 63 | 12 | 25 | 0.907063 | 0.84 | 0.16 | 0.092937 | 1.220194 | 0.642692 | 2.316618 |
| LIHC | SBS19 | Unknown | 232 | 55 | 20 | 37 | 0.862454 | 0.733333 | 0.266667 | 0.137546 | 2.019911 | 1.174668 | 3.473357 |
| LIHC | SBS21 | Mismatchrepair | 228 | 55 | 20 | 41 | 0.847584 | 0.733333 | 0.266667 | 0.152416 | 1.896919 | 1.115125 | 3.226814 |
| LIHC | SBS22 | Aristolochic acid | 185 | 63 | 12 | 84 | 0.687732 | 0.84 | 0.16 | 0.312268 | 0.635987 | 0.338277 | 1.195705 |
| LIHC | SBS23 | Unknown | 242 | 65 | 10 | 27 | 0.899628 | 0.866667 | 0.133333 | 0.100372 | 1.547397 | 0.777011 | 3.081602 |
| LIHC | SBS24 | Aflatoxin | 247 | 61 | 14 | 22 | 0.918216 | 0.813333 | 0.186667 | 0.081784 | 3.583024 | 1.907359 | 6.730806 |
| LIHC | SBS25 | Chemotherapy | 222 | 59 | 16 | 47 | 0.825279 | 0.786667 | 0.213333 | 0.174721 | 1.221011 | 0.671867 | 2.21899 |
| LIHC | SBS26 | Mismatchrepair | 162 | 37 | 38 | 107 | 0.60223 | 0.493333 | 0.506667 | 0.39777 | 1.575241 | 0.999346 | 2.483007 |
| LIHC | SBS28 | Unknown | 174 | 53 | 22 | 95 | 0.64684 | 0.706667 | 0.293333 | 0.35316 | 0.691459 | 0.41394 | 1.155039 |
| LIHC | SBS29 | Tobacco chewing in Hollst | 232 | 69 | 6 | 37 | 0.862454 | 0.92 | 0.08 | 0.137546 | 0.690768 | 0.297299 | 1.604983 |
| LIHC | SBS20 | Mismatchrepair + POLD1mut | 225 | 61 | 14 | 44 | 0.836431 | 0.813333 | 0.186667 | 0.163569 | 1.598865 | 0.88096 | 2.901799 |
| LIHC | SBS30 | BER repair-NTHL1mut | 210 | 57 | 18 | 59 | 0.780669 | 0.76 | 0.24 | 0.219331 | 0.995287 | 0.574617 | 1.723924 |
| LIHC | SBS31 | PLChemotherapy | 201 | 59 | 16 | 68 | 0.747212 | 0.786667 | 0.213333 | 0.252788 | 1.020253 | 0.580073 | 1.794457 |
| LIHC | SBS32 | AZA Chemotherapy | 221 | 69 | 6 | 48 | 0.821561 | 0.92 | 0.08 | 0.178439 | 0.446234 | 0.191015 | 1.042456 |
| LIHC | SBS33 | Unknown | 232 | 69 | 6 | 37 | 0.862454 | 0.92 | 0.08 | 0.137546 | 0.605685 | 0.258415 | 1.419635 |
| LIHC | SBS34 | Unknown | 209 | 48 | 27 | 60 | 0.776952 | 0.64 | 0.36 | 0.223048 | 1.424505 | 0.882181 | 2.300227 |
| LIHC | SBS35 | PLChemotherapy | 237 | 66 | 9 | 32 | 0.881041 | 0.88 | 0.12 | 0.118959 | 1.930625 | 0.937713 | 3.974898 |
| LIHC | SBS36 | BERrepair MUTY | 233 | 65 | 10 | 36 | 0.866171 | 0.866667 | 0.133333 | 0.133829 | 0.890112 | 0.438055 | 1.808676 |
| LIHC | SBS37 | Unknown | 231 | 66 | 9 | 38 | 0.858736 | 0.88 | 0.12 | 0.141264 | 0.737196 | 0.361768 | 1.502227 |
| LIHC | SBS38 | UV | 243 | 61 | 14 | 26 | 0.903346 | 0.813333 | 0.186667 | 0.096654 | 1.514717 | 0.831834 | 2.758204 |
| LIHC | SBS39 | Unknown | 118 | 20 | 55 | 151 | 0.438662 | 0.266667 | 0.733333 | 0.561338 | 2.02942 | 1.197005 | 3.440708 |
| LIHC | SBS42 | Haloalkane | 219 | 65 | 10 | 50 | 0.814126 | 0.866667 | 0.133333 | 0.185874 | 0.699331 | 0.353403 | 1.38387 |
| LIHC | SBS85 | Actind cytidinedeaminase | 258 | 70 | 5 | 11 | 0.959108 | 0.933333 | 0.066667 | 0.040892 | 1.653323 | 0.644525 | 4.241073 |
| LIHC | SBS40 | Unknown | | | | | | | | | | | |
| LIHC | SBS41 | Unknown | | | | | | | | | | | |
| LIHC | SBS44 | Mismatchrepair | | | | | | | | | | | |
| LIHC | SBS7c | UV | | | | | | | | | | | |
| LIHC | SBS84 | Actind cytidinedeaminase | | | | | | | | | | | |
| LIHC | SBS9 | PolETA Somatichypermutati [symbol] | | | | | | | | | | | |
| LUAD | SBS1 | Endogenous clock-like age | 31 | 14 | 94 | 314 | 0.089855 | 0.12963 | 0.87037 | 0.910145 | 0.733877 | 0.417822 | 1.289009 |
| LUAD | SBS2 | Endogenous Apobec3a | 209 | 59 | 49 | 136 | 0.605797 | 0.546296 | 0.453704 | 0.394203 | 1.188411 | 0.809585 | 1.744499 |
| LUAD | SBS3 | Homologous repair | 334 | 105 | 3 | 11 | 0.968116 | 0.972222 | 0.027778 | 0.031884 | 1.335772 | 0.419647 | 4.251875 |
| LUAD | SBS4 | Tobacco | 244 | 84 | 24 | 101 | 0.707246 | 0.777778 | 0.222222 | 0.292754 | 0.573131 | 0.356218 | 0.92213 |
| LUAD | SBS6 | Mismatchrepair | 310 | 92 | 16 | 35 | 0.898551 | 0.851852 | 0.148148 | 0.101449 | 1.715056 | 1.004803 | 2.927357 |
| LUAD | SBS7a | UV | 269 | 76 | 32 | 76 | 0.77971 | 0.703704 | 0.296296 | 0.22029 | 1.296047 | 0.853312 | 1.968492 |
| LUAD | SBS7b | UV | 265 | 86 | 22 | 80 | 0.768116 | 0.796296 | 0.203704 | 0.231884 | 0.812684 | 0.504455 | 1.309244 |
| LUAD | SBS7c | UV | 302 | 103 | 5 | 43 | 0.875362 | 0.953704 | 0.046296 | 0.124638 | 0.343792 | 0.138734 | 0.851939 |
| LUAD | SBS7d | UV | 269 | 89 | 19 | 76 | 0.77971 | 0.824074 | 0.175926 | 0.22029 | 0.633052 | 0.384388 | 1.042579 |
| LUAD | SBS8 | Unknown | 327 | 104 | 4 | 18 | 0.947826 | 0.962963 | 0.037037 | 0.052174 | 0.897159 | 0.328945 | 2.446899 |
| LUAD | SBS10a | Polymerase epsilon hyperm [symbol] | 279 | 94 | 14 | 66 | 0.808696 | 0.87037 | 0.12963 | 0.191304 | 0.630735 | 0.358087 | 1.110975 |
| LUAD | SBS10b | Polymerase epsilon mutati [symbol] | 168 | 39 | 69 | 177 | 0.486957 | 0.361111 | 0.638889 | 0.513043 | 1.529222 | 1.026595 | 2.277939 |
| LUAD | SBS11 | Temozolo Chemotherapy | 327 | 101 | 7 | 18 | 0.947826 | 0.935185 | 0.064815 | 0.052174 | 1.185867 | 0.544163 | 2.584298 |
| LUAD | SBS12 | Liver cancer | 314 | 89 | 19 | 31 | 0.910145 | 0.824074 | 0.175926 | 0.089855 | 1.493464 | 0.90645 | 2.460626 |
| LUAD | SBS13 | Endogenous apobec | 254 | 73 | 35 | 91 | 0.736232 | 0.675926 | 0.324074 | 0.263768 | 1.290129 | 0.860019 | 1.935345 |
| LUAD | SBS14 | Mismatchrepair + PolEpsil | 301 | 92 | 16 | 44 | 0.872464 | 0.851852 | 0.148148 | 0.127536 | 0.886569 | 0.517276 | 1.519509 |
| LUAD | SBS15 | Mismatchrepair | 274 | 97 | 11 | 71 | 0.794203 | 0.898148 | 0.101852 | 0.205797 | 0.533988 | 0.285237 | 0.999671 |
| LUAD | SBS16 | Unknown | 305 | 94 | 14 | 40 | 0.884058 | 0.87037 | 0.12963 | 0.115942 | 0.768642 | 0.4343 | 1.360374 |
| LUAD | SBS17a | Unknown | 294 | 94 | 14 | 51 | 0.852174 | 0.87037 | 0.12963 | 0.147826 | 0.765501 | 0.433921 | 1.350457 |
| LUAD | SBS17b | Unknown | 256 | 66 | 42 | 89 | 0.742029 | 0.611111 | 0.388889 | 0.257971 | 1.638175 | 1.10399 | 2.430836 |
| LUAD | SBS18 | ReactiveOxy | 303 | 92 | 16 | 42 | 0.878261 | 0.851852 | 0.148148 | 0.121739 | 0.983549 | 0.572817 | 1.688791 |
| LUAD | SBS19 | Unknown | 293 | 85 | 23 | 52 | 0.849275 | 0.787037 | 0.212963 | 0.150725 | 1.418974 | 0.888507 | 2.266147 |
| LUAD | SBS21 | Mismatchrepair | 205 | 79 | 29 | 140 | 0.594203 | 0.731481 | 0.268519 | 0.405797 | 0.698761 | 0.452481 | 1.079087 |
| LUAD | SBS22 | Aristolochic acid | 296 | 86 | 22 | 49 | 0.857971 | 0.796296 | 0.203704 | 0.142029 | 1.131754 | 0.70334 | 1.821121 |
| LUAD | SBS23 | Unknown | 311 | 94 | 14 | 34 | 0.901449 | 0.87037 | 0.12963 | 0.098551 | 1.390561 | 0.788633 | 2.451914 |
| LUAD | SBS24 | Aflatoxin | 184 | 68 | 40 | 161 | 0.533333 | 0.62963 | 0.37037 | 0.466667 | 0.635345 | 0.422881 | 0.954554 |
| LUAD | SBS25 | Chemotherapy | 308 | 84 | 24 | 37 | 0.892754 | 0.777778 | 0.222222 | 0.107246 | 1.824833 | 1.147991 | 2.900734 |
| LUAD | SBS26 | Mismatchrepair | 272 | 83 | 25 | 73 | 0.788406 | 0.768519 | 0.231481 | 0.211594 | 1.394711 | 0.887527 | 2.191729 |
| LUAD | SBS28 | Unknown | 311 | 92 | 16 | 34 | 0.901449 | 0.851852 | 0.148148 | 0.098551 | 1.495207 | 0.867465 | 2.577213 |
| LUAD | SBS29 | Tobacco chewing in Hollst | 298 | 85 | 23 | 47 | 0.863768 | 0.787037 | 0.212963 | 0.136232 | 1.65353 | 1.02876 | 2.657726 |
| LUAD | SBS20 | Mismatchrepair + POLD1mut | 274 | 92 | 16 | 71 | 0.794203 | 0.851852 | 0.148148 | 0.205797 | 0.785162 | 0.458368 | 1.344943 |
| LUAD | SBS30 | BER repair-NTHL1mut | 312 | 93 | 15 | 33 | 0.904348 | 0.861111 | 0.138889 | 0.095652 | 1.409251 | 0.791041 | 2.510601 |
| LUAD | SBS31 | PLChemotherapy | 297 | 103 | 5 | 48 | 0.86087 | 0.953704 | 0.046296 | 0.13913 | 0.303261 | 0.123098 | 0.747107 |
| LUAD | SBS32 | AZA Chemotherapy | 290 | 92 | 16 | 55 | 0.84058 | 0.851852 | 0.148148 | 0.15942 | 0.878322 | 0.515459 | 1.496625 |
| LUAD | SBS33 | Unknown | 305 | 89 | 19 | 40 | 0.884058 | 0.824074 | 0.175926 | 0.115942 | 1.544252 | 0.934514 | 2.551823 |
| LUAD | SBS34 | Unknown | 302 | 95 | 13 | 43 | 0.875362 | 0.87963 | 0.12037 | 0.124638 | 0.877711 | 0.490283 | 1.571288 |
| LUAD | SBS35 | PLChemotherapy | 302 | 93 | 15 | 43 | 0.875362 | 0.861111 | 0.138889 | 0.124638 | 1.095492 | 0.628278 | 1.910147 |
| LUAD | SBS36 | BERrepair MUTY | 299 | 96 | 12 | 46 | 0.866667 | 0.888889 | 0.111111 | 0.133333 | 0.913264 | 0.499491 | 1.669803 |
| LUAD | SBS37 | Unknown | 328 | 103 | 5 | 17 | 0.950725 | 0.953704 | 0.046296 | 0.049275 | 1.069958 | 0.432459 | 2.647211 |
| LUAD | SBS38 | UV | 286 | 94 | 14 | 59 | 0.828986 | 0.87037 | 0.12963 | 0.171014 | 0.934917 | 0.528888 | 1.652654 |
| LUAD | SBS39 | Unknown | 221 | 58 | 50 | 124 | 0.64058 | 0.537037 | 0.462963 | 0.35942 | 1.344978 | 0.909636 | 1.988671 |
| LUAD | SBS42 | Haloalkane | 290 | 85 | 23 | 55 | 0.84058 | 0.787037 | 0.212963 | 0.15942 | 1.524832 | 0.95564 | 2.433042 |
| LUAD | SBS84 | Actind cytidinedeaminase | 326 | 99 | 9 | 19 | 0.944928 | 0.916667 | 0.083333 | 0.055072 | 1.48422 | 0.739439 | 2.97916 |
| LUAD | SBS40 | Unknown | | | | | | | | | | | |
| LUAD | SBS41 | Unknown | | | | | | | | | | | |
| LUAD | SBS44 | Mismatchrepair | | | | | | | | | | | |
| LUAD | SBS85 | Actind cytidinedeaminase | | | | | | | | | | | |
| LUAD | SBS9 | PolETA Somatichypermutation | | | | | | | | | | | |
| LUSC | SBS1 | Endogenous clock-like age | 34 | 7 | 80 | 298 | 0.10241 | 0.08046 | 0.91954 | 0.89759 | 1.279408 | 0.588499 | 2.781455 |
| LUSC | SBS2 | Endogenous Apobec3a | 171 | 49 | 38 | 161 | 0.51506 | 0.563218 | 0.436782 | 0.48494 | 0.79799 | 0.519958 | 1.22469 |
| LUSC | SBS3 | Homologous repair | 308 | 85 | 2 | 24 | 0.927711 | 0.977011 | 0.022989 | 0.072289 | 0.448456 | 0.109569 | 1.83549 |
| LUSC | SBS4 | Tobacco | 67 | 25 | 62 | 265 | 0.201807 | 0.287356 | 0.712644 | 0.798193 | 0.694503 | 0.435822 | 1.106721 |
| LUSC | SBS6 | Mismatchrepair | 155 | 49 | 38 | 177 | 0.466867 | 0.563218 | 0.436782 | 0.533133 | 0.678035 | 0.441494 | 1.041309 |
| LUSC | SBS7a | UV | 275 | 73 | 14 | 57 | 0.828313 | 0.83908 | 0.16092 | 0.171687 | 0.792916 | 0.44546 | 1.411386 |
| LUSC | SBS7b | UV | 268 | 75 | 12 | 64 | 0.807229 | 0.862069 | 0.137931 | 0.192771 | 0.44868 | 0.242118 | 0.831471 |
| LUSC | SBS7c | UV | 291 | 75 | 12 | 41 | 0.876506 | 0.862069 | 0.137931 | 0.123494 | 0.963857 | 0.520855 | 1.783644 |
| LUSC | SBS7d | UV | 264 | 71 | 16 | 68 | 0.795181 | 0.816092 | 0.183908 | 0.204819 | 0.772996 | 0.445741 | 1.340516 |
| LUSC | SBS8 | Unknown | 292 | 78 | 9 | 40 | 0.879518 | 0.896552 | 0.103448 | 0.120482 | 0.657873 | 0.323748 | 1.336831 |
| LUSC | SBS10a | Polymerase epsilon hyperm [symbol] | 279 | 78 | 9 | 53 | 0.840361 | 0.896552 | 0.103448 | 0.159639 | 0.490029 | 0.244788 | 0.980966 |
| LUSC | SBS10b | Polymerase epsilon mutati [symbol] | 269 | 73 | 14 | 63 | 0.810241 | 0.83908 | 0.16092 | 0.189759 | 0.833354 | 0.463902 | 1.497038 |
| LUSC | SBS11 | Temozolo Chemotherapy | 308 | 77 | 10 | 24 | 0.927711 | 0.885057 | 0.114943 | 0.072289 | 1.173913 | 0.605772 | 2.274901 |
| LUSC | SBS12 | Liver cancer | 254 | 65 | 22 | 78 | 0.76506 | 0.747126 | 0.252874 | 0.23494 | 1.09692 | 0.670809 | 1.793707 |
| LUSC | SBS13 | Endogenous apobec | 283 | 77 | 10 | 49 | 0.85241 | 0.885057 | 0.114943 | 0.14759 | 0.730095 | 0.373204 | 1.428276 |
| LUSC | SBS14 | Mismatchrepair + PolEpsil | 294 | 82 | 5 | 38 | 0.885542 | 0.942529 | 0.057471 | 0.114458 | 0.474683 | 0.192233 | 1.172142 |
| LUSC | SBS15 | Mismatchrepair | 244 | 68 | 19 | 88 | 0.73494 | 0.781609 | 0.218391 | 0.26506 | 0.833796 | 0.497481 | 1.397471 |
| LUSC | SBS16 | Unknown | 225 | 65 | 22 | 107 | 0.677711 | 0.747126 | 0.252874 | 0.322289 | 0.679955 | 0.417193 | 1.108212 |
| LUSC | SBS17a | Unknown | 179 | 37 | 50 | 153 | 0.539157 | 0.425287 | 0.574713 | 0.460843 | 1.375722 | 0.885895 | 2.136384 |
| LUSC | SBS17b | Unknown | 218 | 61 | 26 | 114 | 0.656627 | 0.701149 | 0.298851 | 0.343373 | 0.756875 | 0.477026 | 1.200898 |
| LUSC | SBS18 | ReactiveOxy | 300 | 73 | 14 | 32 | 0.903614 | 0.83908 | 0.16092 | 0.096386 | 1.372819 | 0.761185 | 2.475918 |
| LUSC | SBS19 | Unknown | 299 | 74 | 13 | 33 | 0.900602 | 0.850575 | 0.149425 | 0.099398 | 1.297718 | 0.715956 | 2.3522 |
| LUSC | SBS21 | Mismatchrepair | 265 | 79 | 8 | 67 | 0.798193 | 0.908046 | 0.091954 | 0.201807 | 0.497314 | 0.239841 | 1.031191 |
| LUSC | SBS22 | Aristolochic acid | 265 | 72 | 15 | 67 | 0.798193 | 0.827586 | 0.172414 | 0.201807 | 0.773138 | 0.435676 | 1.371989 |
| LUSC | SBS23 | Unknown | 291 | 80 | 7 | 41 | 0.876506 | 0.91954 | 0.08046 | 0.123494 | 0.460011 | 0.208587 | 1.014492 |
| LUSC | SBS24 | Aflatoxin | 65 | 26 | 61 | 267 | 0.195783 | 0.298851 | 0.701149 | 0.804217 | 0.505534 | 0.313239 | 0.815875 |
| LUSC | SBS25 | Chemotherapy | 286 | 72 | 15 | 46 | 0.861446 | 0.827586 | 0.172414 | 0.138554 | 1.279828 | 0.720158 | 2.274445 |
| LUSC | SBS26 | Mismatchrepair | 261 | 76 | 11 | 71 | 0.786145 | 0.873563 | 0.126437 | 0.213855 | 0.618706 | 0.325941 | 1.174435 |
| LUSC | SBS28 | Unknown | 277 | 75 | 12 | 55 | 0.834337 | 0.862069 | 0.137931 | 0.165663 | 1.034856 | 0.557789 | 1.919949 |
| LUSC | SBS29 | Tobacco chewing in Hollst | 259 | 58 | 29 | 73 | 0.78012 | 0.666667 | 0.333333 | 0.21988 | 1.335585 | 0.851907 | 2.093874 |
| LUSC | SBS20 | Mismatchrepair + POLD1mut | 256 | 59 | 28 | 76 | 0.771084 | 0.678161 | 0.321839 | 0.228916 | 1.294239 | 0.821962 | 2.037874 |
| LUSC | SBS30 | BER repair-NTHL1mut | 300 | 75 | 12 | 32 | 0.903614 | 0.862069 | 0.137931 | 0.096386 | 1.281303 | 0.690256 | 2.378447 |
| LUSC | SBS31 | PLChemotherapy | 256 | 69 | 18 | 76 | 0.771084 | 0.793103 | 0.206897 | 0.228916 | 0.912378 | 0.541755 | 1.536549 |
| LUSC | SBS32 | AZA Chemotherapy | 291 | 85 | 2 | 41 | 0.876506 | 0.977011 | 0.022989 | 0.123494 | 0.189726 | 0.046266 | 0.778019 |
| LUSC | SBS33 | Unknown | 181 | 63 | 24 | 151 | 0.545181 | 0.724138 | 0.275862 | 0.454819 | 0.514801 | 0.320443 | 0.827044 |
| LUSC | SBS34 | Unknown | 293 | 71 | 16 | 39 | 0.88253 | 0.816092 | 0.183908 | 0.11747 | 1.272937 | 0.73645 | 2.200243 |
| LUSC | SBS35 | PLChemotherapy | 294 | 79 | 8 | 38 | 0.885542 | 0.908046 | 0.091954 | 0.114458 | 0.932935 | 0.44539 | 1.954172 |
| LUSC | SBS36 | BERrepair MUTY | 289 | 80 | 7 | 43 | 0.870482 | 0.91954 | 0.08046 | 0.129518 | 0.429341 | 0.193442 | 0.952916 |
| LUSC | SBS37 | Unknown | 320 | 82 | 5 | 12 | 0.963855 | 0.942529 | 0.057471 | 0.036145 | 1.709397 | 0.669463 | 4.364747 |
| LUSC | SBS38 | UV | 274 | 63 | 24 | 58 | 0.825301 | 0.724138 | 0.275862 | 0.174699 | 1.457552 | 0.902748 | 2.353324 |
| LUSC | SBS39 | Unknown | 49 | 16 | 71 | 283 | 0.14759 | 0.183908 | 0.816092 | 0.85241 | 0.529466 | 0.302905 | 0.925485 |
| LUSC | SBS42 | Haloalkane | 294 | 79 | 8 | 38 | 0.885542 | 0.908046 | 0.091954 | 0.114458 | 0.84702 | 0.406289 | 1.765846 |
| LUSC | SBS84 | Actind cytidinedeaminase | 306 | 80 | 7 | 26 | 0.921687 | 0.91954 | 0.08046 | 0.078313 | 0.93188 | 0.428123 | 2.028389 |
| LUSC | SBS85 | Actind cytidinedeaminase | 320 | 84 | 3 | 12 | 0.963855 | 0.965517 | 0.034483 | 0.036145 | 1.841739 | 0.568644 | 5.965074 |
| LUSC | SBS40 | Unknown | | | | | | | | | | | |
| LUSC | SBS41 | Unknown | | | | | | | | | | | |
| LUSC | SBS44 | Mismatchrepair | | | | | | | | | | | |
| LUSC | SBS9 | PolETA Somatichypermutation | | | | | | | | | | | |
| OV | SBS1 | Endogenous clock-like age | 114 | 98 | 69 | 58 | 0.662791 | 0.586826 | 0.413174 | 0.337209 | 1.489411 | 1.075751 | 2.062137 |
| OV | SBS2 | Endogenous Apobec3a | 158 | 144 | 23 | 14 | 0.918605 | 0.862275 | 0.137725 | 0.081395 | 1.222058 | 0.784071 | 1.904708 |
| OV | SBS3 | Homologous repair | 151 | 146 | 21 | 21 | 0.877907 | 0.874251 | 0.125749 | 0.122093 | 1.139907 | 0.719926 | 1.80489 |
| OV | SBS4 | Tobacco | 107 | 116 | 51 | 65 | 0.622093 | 0.694611 | 0.305389 | 0.377907 | 0.793011 | 0.567107 | 1.108901 |
| OV | SBS6 | Mismatchrepair | 140 | 133 | 34 | 32 | 0.813953 | 0.796407 | 0.203593 | 0.186047 | 0.769589 | 0.524927 | 1.128284 |
| OV | SBS7a | UV | 87 | 113 | 54 | 85 | 0.505814 | 0.676647 | 0.323353 | 0.494186 | 0.56687 | 0.407376 | 0.788809 |
| OV | SBS7b | UV | 117 | 122 | 45 | 55 | 0.680233 | 0.730539 | 0.269461 | 0.319767 | 0.785691 | 0.55564 | 1.110988 |
| OV | SBS7c | UV | 133 | 137 | 30 | 39 | 0.773256 | 0.820359 | 0.179641 | 0.226744 | 0.747078 | 0.500733 | 1.114617 |
| OV | SBS7d | UV | 142 | 138 | 29 | 30 | 0.825581 | 0.826347 | 0.173653 | 0.174419 | 1.113194 | 0.739564 | 1.675581 |
| OV | SBS8 | Unknown | 150 | 152 | 15 | 22 | 0.872093 | 0.91018 | 0.08982 | 0.127907 | 0.549396 | 0.320947 | 0.940456 |
| OV | SBS9 | PolETA Somatichypermutation | 156 | 148 | 19 | 16 | 0.906977 | 0.886228 | 0.113772 | 0.093023 | 0.787717 | 0.484758 | 1.280016 |
| OV | SBS10a | Polymerase epsilon hyperm [symbol] | 157 | 145 | 22 | 15 | 0.912791 | 0.868263 | 0.131737 | 0.087209 | 1.387545 | 0.881593 | 2.183867 |
| OV | SBS10b | Polymerase epsilon mutati [symbol] | 151 | 131 | 36 | 21 | 0.877907 | 0.784431 | 0.215569 | 0.122093 | 1.523096 | 1.044954 | 2.220023 |
| OV | SBS11 | Temozolo Chemotherapy | 148 | 139 | 28 | 24 | 0.860465 | 0.832335 | 0.167665 | 0.139535 | 1.216588 | 0.808026 | 1.831731 |
| OV | SBS12 | Liver cancer | 150 | 154 | 13 | 22 | 0.872093 | 0.922156 | 0.077844 | 0.127907 | 0.637161 | 0.360135 | 1.127284 |
| OV | SBS13 | Endogenous apobec | 94 | 98 | 69 | 78 | 0.546512 | 0.586826 | 0.413174 | 0.453488 | 0.691941 | 0.503972 | 0.950016 |
| OV | SBS14 | Mismatchrepair + PolEpsil | 154 | 149 | 18 | 18 | 0.895349 | 0.892216 | 0.107784 | 0.104651 | 1.265418 | 0.763538 | 2.097186 |
| OV | SBS15 | Mismatchrepair | 107 | 109 | 58 | 65 | 0.622093 | 0.652695 | 0.347305 | 0.377907 | 0.868107 | 0.628945 | 1.198211 |
| OV | SBS16 | Unknown | 126 | 123 | 44 | 46 | 0.732558 | 0.736527 | 0.263473 | 0.267442 | 1.388362 | 0.97953 | 1.96783 |
| OV | SBS17a | Unknown | 96 | 94 | 73 | 76 | 0.55814 | 0.562874 | 0.437126 | 0.44186 | 0.885088 | 0.650451 | 1.204367 |
| OV | SBS17b | Unknown | 128 | 139 | 28 | 44 | 0.744186 | 0.832335 | 0.167665 | 0.255814 | 0.578189 | 0.383462 | 0.8718 |
| OV | SBS18 | ReactiveOxy | 134 | 142 | 25 | 38 | 0.77907 | 0.850299 | 0.149701 | 0.22093 | 1.168964 | 0.761607 | 1.7942 |
| OV | SBS19 | Unknown | 151 | 143 | 24 | 21 | 0.877907 | 0.856287 | 0.143713 | 0.122093 | 0.839628 | 0.543587 | 1.296895 |
| OV | SBS21 | Mismatchrepair | 120 | 104 | 63 | 52 | 0.697674 | 0.622754 | 0.377246 | 0.302326 | 1.375226 | 1.00325 | 1.885119 |
| OV | SBS22 | Aristolochic acid | 87 | 79 | 88 | 85 | 0.505814 | 0.473054 | 0.526946 | 0.494186 | 1.22764 | 0.90199 | 1.670862 |
| OV | SBS23 | Unknown | 145 | 145 | 22 | 27 | 0.843023 | 0.868263 | 0.131737 | 0.156977 | 1.359488 | 0.865068 | 2.13649 |
| OV | SBS24 | Aflatoxin | 90 | 73 | 94 | 82 | 0.523256 | 0.437126 | 0.562874 | 0.476744 | 1.224458 | 0.897989 | 1.669617 |
| OV | SBS25 | Chemotherapy | 127 | 124 | 43 | 45 | 0.738372 | 0.742515 | 0.257485 | 0.261628 | 1.086582 | 0.764293 | 1.544776 |
| OV | SBS26 | Mismatchrepair | 138 | 123 | 44 | 34 | 0.802326 | 0.736527 | 0.263473 | 0.197674 | 1.141573 | 0.807986 | 1.612885 |
| OV | SBS28 | Unknown | 134 | 138 | 29 | 38 | 0.77907 | 0.826347 | 0.173653 | 0.22093 | 0.763044 | 0.508438 | 1.145146 |
| OV | SBS29 | Tobacco chewing in Hollst | 99 | 105 | 62 | 73 | 0.575581 | 0.628743 | 0.371257 | 0.424419 | 0.750038 | 0.545618 | 1.031045 |
| OV | SBS20 | Mismatchrepair + POLD1mut | 127 | 117 | 50 | 45 | 0.738372 | 0.700599 | 0.299401 | 0.261628 | 1.478086 | 1.055361 | 2.070134 |
| OV | SBS30 | BER repair-NTHL1mut | 110 | 100 | 67 | 62 | 0.639535 | 0.598802 | 0.401198 | 0.360465 | 1.368702 | 1.001678 | 1.870208 |
| OV | SBS31 | PLChemotherapy | 143 | 146 | 21 | 29 | 0.831395 | 0.874251 | 0.125749 | 0.168605 | 0.820759 | 0.51829 | 1.299748 |
| OV | SBS32 | AZA Chemotherapy | 148 | 151 | 16 | 24 | 0.860465 | 0.904192 | 0.095808 | 0.139535 | 0.622446 | 0.369844 | 1.047576 |
| OV | SBS33 | Unknown | 119 | 125 | 42 | 53 | 0.69186 | 0.748503 | 0.251497 | 0.30814 | 0.903095 | 0.634072 | 1.286259 |
| OV | SBS34 | Unknown | 155 | 149 | 18 | 17 | 0.901163 | 0.892216 | 0.107784 | 0.098837 | 0.738461 | 0.450235 | 1.2112 |
| OV | SBS35 | PLChemotherapy | 153 | 151 | 16 | 19 | 0.889535 | 0.904192 | 0.095808 | 0.110465 | 0.739759 | 0.438684 | 1.247465 |
| OV | SBS36 | BERrepair MUTY | 156 | 148 | 19 | 16 | 0.906977 | 0.886228 | 0.113772 | 0.093023 | 0.778159 | 0.479685 | 1.262354 |
| OV | SBS37 | Unknown | 123 | 103 | 64 | 49 | 0.715116 | 0.616766 | 0.383234 | 0.284884 | 1.36656 | 0.995802 | 1.87536 |
| OV | SBS38 | UV | 111 | 118 | 49 | 61 | 0.645349 | 0.706587 | 0.293413 | 0.354651 | 0.754112 | 0.539158 | 1.054763 |
| OV | SBS39 | Unknown | 56 | 75 | 92 | 116 | 0.325581 | 0.449102 | 0.550898 | 0.674419 | 0.554464 | 0.404336 | 0.760335 |
| OV | SBS42 | Haloalkane | 148 | 144 | 23 | 24 | 0.860465 | 0.862275 | 0.137725 | 0.139535 | 0.962731 | 0.609035 | 1.521837 |
| OV | SBS84 | Actind cytidinedeaminase | 124 | 134 | 33 | 48 | 0.72093 | 0.802395 | 0.197605 | 0.27907 | 0.735027 | 0.499287 | 1.082071 |
| OV | SBS85 | Actind cytidinedeaminase | 162 | 153 | 14 | 10 | 0.94186 | 0.916168 | 0.083832 | 0.05814 | 1.428881 | 0.820506 | 2.488343 |
| OV | SBS40 | Unknown | | | | | | | | | | | |
| OV | SBS41 | Unknown | | | | | | | | | | | |
| OV | SBS44 | Mismatchrepair | | | | | | | | | | | |
| PAAD | SBS1 | Endogenous clock-like age | 21 | 9 | 68 | 68 | 0.235955 | 0.116883 | 0.883117 | 0.764045 | 1.603868 | 0.784975 | 3.277038 |
| PAAD | SBS2 | Endogenous Apobec3a | 58 | 39 | 38 | 31 | 0.651685 | 0.506494 | 0.493506 | 0.348315 | 1.063906 | 0.665894 | 1.699814 |
| PAAD | SBS3 | Homologous repair | 88 | 77 | 0 | 1 | 0.988764 | 1 | 0 | 0.011236 | 4.41E−07 | 0 | |
| PAAD | SBS4 | Tobacco | 81 | 67 | 10 | 8 | 0.910112 | 0.87013 | 0.12987 | 0.089888 | 1.382913 | 0.702847 | 2.721003 |
| PAAD | SBS6 | Mismatchrepair | 64 | 47 | 30 | 25 | 0.719101 | 0.61039 | 0.38961 | 0.280899 | 1.253116 | 0.778242 | 2.017751 |
| PAAD | SBS7a | UV | 78 | 62 | 15 | 11 | 0.876404 | 0.805195 | 0.194805 | 0.123596 | 1.209366 | 0.674163 | 2.169455 |
| PAAD | SBS7b | UV | 75 | 60 | 17 | 14 | 0.842697 | 0.779221 | 0.220779 | 0.157303 | 1.199749 | 0.694626 | 2.072191 |
| PAAD | SBS7c | UV | 75 | 72 | 5 | 14 | 0.842697 | 0.935065 | 0.064935 | 0.157303 | 0.446106 | 0.178089 | 1.11748 |
| PAAD | SBS7d | UV | 65 | 63 | 14 | 24 | 0.730337 | 0.818182 | 0.181818 | 0.269663 | 0.716118 | 0.394436 | 1.300146 |
| PAAD | SBS8 | Unknown | 73 | 74 | 3 | 16 | 0.820225 | 0.961039 | 0.038961 | 0.179775 | 0.178745 | 0.051427 | 0.621259 |
| PAAD | SBS9 | PolETA Somatichypermutation | 88 | 76 | 1 | 1 | 0.988764 | 0.987013 | 0.012987 | 0.011236 | 2.053762 | 0.273648 | 15.41376 |
| PAAD | SBS10a | Polymerase epsilon hyperm [symbol] | 74 | 60 | 17 | 15 | 0.831461 | 0.779221 | 0.220779 | 0.168539 | 0.937699 | 0.540165 | 1.627799 |
| PAAD | SBS10b | Polymerase epsilon mutati [symbol] | 59 | 45 | 32 | 30 | 0.662921 | 0.584416 | 0.415584 | 0.337079 | 1.230313 | 0.759816 | 1.992154 |
| PAAD | SBS11 | Temozolo Chemotherapy | 77 | 70 | 7 | 12 | 0.865169 | 0.909091 | 0.090909 | 0.134831 | 0.703435 | 0.321589 | 1.538678 |
| PAAD | SBS12 | Liver cancer | 72 | 67 | 10 | 17 | 0.808989 | 0.87013 | 0.12987 | 0.191011 | 0.557962 | 0.28222 | 1.103116 |
| PAAD | SBS13 | Endogenous apobec | 52 | 36 | 41 | 37 | 0.58427 | 0.467532 | 0.532468 | 0.41573 | 1.181033 | 0.742451 | 1.878696 |
| PAAD | SBS14 | Mismatchrepair + PolEpsil | 73 | 68 | 9 | 16 | 0.820225 | 0.883117 | 0.116883 | 0.179775 | 0.761522 | 0.373568 | 1.552369 |
| PAAD | SBS15 | Mismatchrepair | 70 | 48 | 29 | 19 | 0.786517 | 0.623377 | 0.376623 | 0.213483 | 1.435183 | 0.893575 | 2.305068 |
| PAAD | SBS16 | Unknown | 84 | 64 | 13 | 5 | 0.94382 | 0.831169 | 0.168831 | 0.05618 | 1.413855 | 0.765758 | 2.610466 |
| PAAD | SBS17a | Unknown | 76 | 55 | 22 | 13 | 0.853933 | 0.714286 | 0.285714 | 0.146067 | 1.995268 | 1.172958 | 3.394066 |
| PAAD | SBS17b | Unknown | 79 | 60 | 17 | 10 | 0.88764 | 0.779221 | 0.220779 | 0.11236 | 1.464147 | 0.839251 | 2.554331 |
| PAAD | SBS18 | ReactiveOxy | 78 | 67 | 10 | 11 | 0.876404 | 0.87013 | 0.12987 | 0.123596 | 1.310043 | 0.665369 | 2.57934 |
| PAAD | SBS19 | Unknown | 69 | 63 | 14 | 20 | 0.775281 | 0.818182 | 0.181818 | 0.224719 | 0.983287 | 0.528394 | 1.829794 |
| PAAD | SBS21 | Mismatchrepair | 72 | 65 | 12 | 17 | 0.808989 | 0.844156 | 0.155844 | 0.191011 | 0.607175 | 0.326186 | 1.13022 |
| PAAD | SBS22 | Aristolochic acid | 78 | 67 | 10 | 11 | 0.876404 | 0.87013 | 0.12987 | 0.123596 | 0.734891 | 0.365575 | 1.477303 |
| PAAD | SBS23 | Unknown | 85 | 71 | 6 | 4 | 0.955056 | 0.922078 | 0.077922 | 0.044944 | 1.289827 | 0.529696 | 3.140771 |
| PAAD | SBS24 | Aflatoxin | 79 | 58 | 19 | 10 | 0.88764 | 0.753247 | 0.246753 | 0.11236 | 1.372473 | 0.80523 | 2.339308 |
| PAAD | SBS25 | Chemotherapy | 87 | 76 | 1 | 2 | 0.977528 | 0.987013 | 0.012987 | 0.022472 | 1.901406 | 0.255662 | 14.14109 |
| PAAD | SBS26 | Mismatchrepair | 82 | 66 | 11 | 7 | 0.921348 | 0.857143 | 0.142857 | 0.078652 | 1.715602 | 0.88974 | 3.308033 |
| PAAD | SBS28 | Unknown | 81 | 65 | 12 | 8 | 0.910112 | 0.844156 | 0.155844 | 0.089888 | 1.223074 | 0.652669 | 2.291987 |
| PAAD | SBS29 | Tobacco chewing in Hollst | 59 | 49 | 28 | 30 | 0.662921 | 0.636364 | 0.363636 | 0.337079 | 0.995468 | 0.618291 | 1.602735 |
| PAAD | SBS20 | Mismatchrepair + POLD1mut | 79 | 69 | 8 | 10 | 0.88764 | 0.896104 | 0.103896 | 0.11236 | 1.074822 | 0.510595 | 2.262541 |
| PAAD | SBS30 | BER repair-NTHL1mut | 73 | 54 | 23 | 16 | 0.820225 | 0.701299 | 0.298701 | 0.179775 | 1.172404 | 0.700396 | 1.962506 |
| PAAD | SBS31 | PLChemotherapy | 79 | 69 | 8 | 10 | 0.88764 | 0.896104 | 0.103896 | 0.11236 | 0.660311 | 0.301263 | 1.447273 |
| PAAD | SBS32 | AZA Chemotherapy | 77 | 71 | 6 | 12 | 0.865169 | 0.922078 | 0.077922 | 0.134831 | 0.595623 | 0.240788 | 1.473357 |
| PAAD | SBS33 | Unknown | 82 | 65 | 12 | 7 | 0.921348 | 0.844156 | 0.155844 | 0.078652 | 1.41726 | 0.738242 | 2.720822 |
| PAAD | SBS34 | Unknown | 79 | 65 | 12 | 10 | 0.88764 | 0.844156 | 0.155844 | 0.11236 | 1.536612 | 0.811448 | 2.909833 |
| PAAD | SBS35 | PLChemotherapy | 78 | 70 | 7 | 11 | 0.876404 | 0.909091 | 0.090909 | 0.123596 | 0.687796 | 0.312022 | 1.516121 |
| PAAD | SBS36 | BERrepair MUTY | 70 | 67 | 10 | 19 | 0.786517 | 0.87013 | 0.12987 | 0.213483 | 0.785028 | 0.391195 | 1.575351 |
| PAAD | SBS37 | Unknown | 80 | 68 | 9 | 9 | 0.898876 | 0.883117 | 0.116883 | 0.101124 | 0.852752 | 0.421758 | 1.724182 |
| PAAD | SBS38 | UV | 76 | 62 | 15 | 13 | 0.853933 | 0.805195 | 0.194805 | 0.146067 | 1.15544 | 0.64822 | 2.059549 |
| PAAD | SBS39 | Unknown | 66 | 62 | 15 | 23 | 0.741573 | 0.805195 | 0.194805 | 0.258427 | 0.577062 | 0.319624 | 1.041852 |
| PAAD | SBS39 | Unknown | 66 | 62 | 15 | 23 | 0.741573 | 0.805195 | 0.194805 | 0.258427 | 0.577062 | 0.319624 | 1.041852 |
| PAAD | SBS42 | Haloalkane | 82 | 65 | 12 | 7 | 0.921348 | 0.844156 | 0.155844 | 0.078652 | 2.009717 | 1.058165 | 3.816951 |
| PAAD | SBS84 | Actind cytidinedeaminase | 82 | 75 | 2 | 7 | 0.921348 | 0.974026 | 0.025974 | 0.078652 | 0.697663 | 0.168587 | 2.887142 |
| PAAD | SBS85 | Actind cytidinedeaminase | 80 | 69 | 8 | 9 | 0.898876 | 0.896104 | 0.103896 | 0.101124 | 0.815248 | 0.380929 | 1.744761 |
| PAAD | SBS40 | Unknown | | | | | | | | | | | |
| PAAD | SBS41 | Unknown | | | | | | | | | | | |
| PAAD | SBS44 | Mismatchrepair | | | | | | | | | | | |
| SARC | SBS1 | Endogenous clock-like age | 138 | 69 | 2 | 23 | 0.857143 | 0.971831 | 0.028169 | 0.142857 | 0.15965 | 0.038532 | 0.661487 |
| SARC | SBS2 | Endogenous Apobec3a | 135 | 54 | 17 | 26 | 0.838509 | 0.760563 | 0.239437 | 0.161491 | 1.184972 | 0.676195 | 2.076559 |
| SARC | SBS3 | Homologous repair | 143 | 60 | 11 | 18 | 0.888199 | 0.84507 | 0.15493 | 0.111801 | 0.992807 | 0.517246 | 1.905603 |
| SARC | SBS4 | Tobacco | 112 | 54 | 17 | 49 | 0.695652 | 0.760563 | 0.239437 | 0.304348 | 0.634815 | 0.364092 | 1.106838 |
| SARC | SBS6 | Mismatchrepair | 119 | 49 | 22 | 42 | 0.73913 | 0.690141 | 0.309859 | 0.26087 | 1.392156 | 0.834756 | 2.321756 |
| SARC | SBS7a | UV | 110 | 57 | 14 | 51 | 0.68323 | 0.802817 | 0.197183 | 0.31677 | 0.664181 | 0.369659 | 1.193361 |
| SARC | SBS7b | UV | 112 | 55 | 16 | 49 | 0.695652 | 0.774648 | 0.225352 | 0.304348 | 0.693083 | 0.394923 | 1.21635 |
| SARC | SBS7c | UV | 133 | 57 | 14 | 28 | 0.826087 | 0.802817 | 0.197183 | 0.173913 | 1.187036 | 0.65378 | 2.155242 |
| SARC | SBS7d | UV | 132 | 53 | 18 | 29 | 0.819876 | 0.746479 | 0.253521 | 0.180124 | 1.744033 | 0.997159 | 3.050318 |
| SARC | SBS8 | Unknown | 137 | 63 | 8 | 24 | 0.850932 | 0.887324 | 0.112676 | 0.149068 | 0.735581 | 0.350529 | 1.543609 |
| SARC | SBS9 | PolETA Somatichypermutation | 155 | 70 | 1 | 6 | 0.962733 | 0.985915 | 0.014085 | 0.037267 | 0.853992 | 0.117746 | 6.193871 |
| SARC | SBS10a | Polymerase epsilon hyperm [symbol] | 140 | 65 | 6 | 21 | 0.869565 | 0.915493 | 0.084507 | 0.130435 | 0.648344 | 0.280057 | 1.500948 |
| SARC | SBS10b | Polymerase epsilon mutati [symbol] | 121 | 41 | 30 | 40 | 0.751553 | 0.577465 | 0.422535 | 0.248447 | 1.626911 | 1.010595 | 2.61909 |
| SARC | SBS11 | Temozolo Chemotherapy | 134 | 66 | 5 | 27 | 0.832298 | 0.929577 | 0.070423 | 0.167702 | 0.396038 | 0.158834 | 0.987483 |
| SARC | SBS12 | Liver cancer | 141 | 66 | 5 | 20 | 0.875776 | 0.929577 | 0.070423 | 0.124224 | 0.587305 | 0.236119 | 1.460817 |
| SARC | SBS13 | Endogenous apobec | 140 | 58 | 13 | 21 | 0.869565 | 0.816901 | 0.183099 | 0.130435 | 1.419913 | 0.768776 | 2.622549 |
| SARC | SBS14 | Mismatchrepair + PolEpsil | 118 | 58 | 13 | 43 | 0.732919 | 0.816901 | 0.183099 | 0.267081 | 0.612102 | 0.333885 | 1.122149 |
| SARC | SBS15 | Mismatchrepair | 141 | 65 | 6 | 20 | 0.875776 | 0.915493 | 0.084507 | 0.124224 | 0.679326 | 0.293611 | 1.571754 |
| SARC | SBS16 | Unknown | 140 | 55 | 16 | 21 | 0.869565 | 0.774648 | 0.225352 | 0.130435 | 1.679573 | 0.960448 | 2.937136 |
| SARC | SBS17a | Unknown | 104 | 48 | 23 | 57 | 0.645963 | 0.676056 | 0.323944 | 0.354037 | 0.792763 | 0.478056 | 1.314643 |
| SARC | SBS17b | Unknown | 137 | 57 | 14 | 24 | 0.850932 | 0.802817 | 0.197183 | 0.149068 | 1.558552 | 0.860717 | 2.822165 |
| SARC | SBS18 | ReactiveOxy | 142 | 64 | 7 | 19 | 0.881988 | 0.901408 | 0.098592 | 0.118012 | 0.73969 | 0.337271 | 1.622262 |
| SARC | SBS19 | Unknown | 102 | 37 | 34 | 59 | 0.63354 | 0.521127 | 0.478873 | 0.36646 | 1.444351 | 0.90362 | 2.30866 |
| SARC | SBS21 | Mismatchrepair | 145 | 62 | 9 | 16 | 0.900621 | 0.873239 | 0.126761 | 0.099379 | 1.387044 | 0.688313 | 2.795082 |
| SARC | SBS22 | Aristolochic acid | 120 | 40 | 31 | 41 | 0.745342 | 0.56338 | 0.43662 | 0.254658 | 2.043352 | 1.275126 | 3.27441 |
| SARC | SBS23 | Unknown | 141 | 62 | 9 | 20 | 0.875776 | 0.873239 | 0.126761 | 0.124224 | 0.803231 | 0.397322 | 1.623821 |
| SARC | SBS24 | Aflatoxin | 98 | 36 | 35 | 63 | 0.608696 | 0.507042 | 0.492958 | 0.391304 | 1.443093 | 0.905187 | 2.300647 |
| SARC | SBS25 | Chemotherapy | 150 | 69 | 2 | 11 | 0.931677 | 0.971831 | 0.028169 | 0.068323 | 0.429445 | 0.104555 | 1.763892 |
| SARC | SBS26 | Mismatchrepair | 136 | 59 | 12 | 25 | 0.84472 | 0.830986 | 0.169014 | 0.15528 | 1.050773 | 0.563943 | 1.957866 |
| SARC | SBS28 | Unknown | 141 | 65 | 6 | 20 | 0.875776 | 0.915493 | 0.084507 | 0.124224 | 0.751723 | 0.323086 | 1.749029 |
| SARC | SBS29 | Tobacco chewing in Hollst | 124 | 54 | 17 | 37 | 0.770186 | 0.760563 | 0.239437 | 0.229814 | 1.185889 | 0.681574 | 2.063361 |
| SARC | SBS22 | Mismatchrepair + POLD1mut | 124 | 62 | 9 | 37 | 0.770186 | 0.873239 | 0.126761 | 0.229814 | 0.462591 | 0.229072 | 0.934164 |
| SARC | SBS30 | BER repair-NTHL1mut | 111 | 42 | 29 | 50 | 0.689441 | 0.591549 | 0.408451 | 0.310559 | 1.431514 | 0.885935 | 2.313073 |
| SARC | SBS31 | PLChemotherapy | 135 | 55 | 16 | 26 | 0.838509 | 0.774648 | 0.225352 | 0.161491 | 1.306709 | 0.734321 | 2.32526 |
| SARC | SBS32 | AZA Chemotherapy | 111 | 45 | 26 | 50 | 0.689441 | 0.633803 | 0.366197 | 0.310559 | 1.202873 | 0.738976 | 1.957982 |
| SARC | SBS33 | Unknown | 135 | 62 | 9 | 26 | 0.838509 | 0.873239 | 0.126761 | 0.161491 | 0.8116 | 0.401682 | 1.63984 |
| SARC | SBS34 | Unknown | 145 | 60 | 11 | 16 | 0.900621 | 0.84507 | 0.15493 | 0.099379 | 1.372633 | 0.718951 | 2.620652 |
| SARC | SBS35 | PLChemotherapy | 138 | 64 | 7 | 23 | 0.857143 | 0.901408 | 0.098592 | 0.142857 | 0.633165 | 0.288065 | 1.391694 |
| SARC | SBS36 | BERrepair MUTY | 141 | 62 | 9 | 20 | 0.875776 | 0.873239 | 0.126761 | 0.124224 | 0.780451 | 0.384495 | 1.584167 |
| SARC | SBS37 | Unknown | 136 | 56 | 15 | 25 | 0.84472 | 0.788732 | 0.211268 | 0.15528 | 1.355318 | 0.764497 | 2.40274 |
| SARC | SBS38 | UV | 123 | 46 | 25 | 38 | 0.763975 | 0.647887 | 0.352113 | 0.236025 | 1.650469 | 1.011447 | 2.693218 |
| SARC | SBS39 | Unknown | 66 | 17 | 54 | 95 | 0.409938 | 0.239437 | 0.760563 | 0.590062 | 1.901288 | 1.092687 | 3.308263 |
| SARC | SBS42 | Haloalkane | 140 | 57 | 14 | 21 | 0.869565 | 0.802817 | 0.197183 | 0.130435 | 1.627957 | 0.905034 | 2.928338 |
| SARC | SBS84 | Actind cytidinedeaminase | 145 | 57 | 14 | 16 | 0.900621 | 0.802817 | 0.197183 | 0.099379 | 1.810656 | 0.996734 | 3.289219 |
| SARC | SBS85 | Actind cytidinedeaminase | 133 | 64 | 7 | 28 | 0.826087 | 0.901408 | 0.098592 | 0.173913 | 0.673044 | 0.3028 | 1.495998 |
| SARC | SBS40 | Unknown | | | | | | | | | | | |
| SARC | SBS41 | Unknown | | | | | | | | | | | |
| SARC | SBS44 | Mismatchrepair | | | | | | | | | | | |
| SKCM | SBS1 | Endogenous clock-like age | 142 | 104 | 82 | 116 | 0.550388 | 0.55914 | 0.44086 | 0.449612 | 0.682458 | 0.505569 | 0.921237 |
| SKCM | SBS2 | Endogenous Apobec3a | 236 | 160 | 26 | 22 | 0.914729 | 0.860215 | 0.139785 | 0.085271 | 1.492814 | 0.975604 | 2.284221 |
| SKCM | SBS3 | Homologous repair | 254 | 181 | 5 | 4 | 0.984496 | 0.973118 | 0.026882 | 0.015504 | 1.323059 | 0.53088 | 3.297328 |
| SKCM | SBS4 | Tobacco | 245 | 169 | 17 | 13 | 0.949612 | 0.908602 | 0.091398 | 0.050388 | 1.947576 | 1.168027 | 3.247402 |
| SKCM | SBS6 | Mismatchrepair | 168 | 128 | 58 | 90 | 0.651163 | 0.688172 | 0.311828 | 0.348837 | 0.775711 | 0.562587 | 1.069573 |
| SKCM | SBS7a | UV | 67 | 72 | 114 | 191 | 0.25969 | 0.387097 | 0.612903 | 0.74031 | 0.526015 | 0.385888 | 0.717026 |
| SKCM | SBS7b | UV | 51 | 51 | 135 | 207 | 0.197674 | 0.274194 | 0.725806 | 0.802326 | 0.450604 | 0.318166 | 0.638169 |
| SKCM | SBS7c | UV | 135 | 104 | 82 | 123 | 0.523256 | 0.55914 | 0.44086 | 0.476744 | 0.660867 | 0.488083 | 0.894817 |
| SKCM | SBS7d | UV | 178 | 104 | 82 | 80 | 0.689922 | 0.55914 | 0.44086 | 0.310078 | 1.449075 | 1.07895 | 1.946168 |
| SKCM | SBS8 | Unknown | 258 | 185 | 1 | 0 | 1 | 0.994624 | 0.005376 | 0 | 5.686784 | 0.690582 | 46.82935 |
| SKCM | SBS9 | PolETA Somatichypermutation | 257 | 185 | 1 | 1 | 0.996124 | 0.994624 | 0.005376 | 0.003876 | 0.488467 | 0.064537 | 3.697121 |
| SKCM | SBS10a | Polymerase epsilon hyperm [symbol] | 255 | 183 | 3 | 3 | 0.988372 | 0.983871 | 0.016129 | 0.011628 | 2.052614 | 0.650623 | 6.47567 |
| SKCM | SBS10b | Polymerase epsilon mutati [symbol] | 111 | 97 | 89 | 147 | 0.430233 | 0.521505 | 0.478495 | 0.569767 | 0.611344 | 0.451711 | 0.82739 |
| SKCM | SBS11 | Temozolo Chemotherapy | 226 | 160 | 26 | 32 | 0.875969 | 0.860215 | 0.139785 | 0.124031 | 0.96235 | 0.632033 | 1.4653 |
| SKCM | SBS12 | Liver cancer | 253 | 177 | 9 | 5 | 0.98062 | 0.951613 | 0.048387 | 0.01938 | 1.362379 | 0.690655 | 2.687418 |
| SKCM | SBS13 | Endogenous apobec | 200 | 127 | 59 | 58 | 0.775194 | 0.682796 | 0.317204 | 0.224806 | 1.625223 | 1.176426 | 2.245233 |
| SKCM | SBS14 | Mismatchrepair + PolEpsil | 250 | 180 | 6 | 8 | 0.968992 | 0.967742 | 0.032258 | 0.031008 | 0.608634 | 0.268454 | 1.379885 |
| SKCM | SBS15 | Mismatchrepair | 230 | 165 | 21 | 28 | 0.891473 | 0.887097 | 0.112903 | 0.108527 | 1.191855 | 0.743525 | 1.910518 |
| SKCM | SBS16 | Unknown | 256 | 178 | 8 | 2 | 0.992248 | 0.956989 | 0.043011 | 0.007752 | 1.351077 | 0.660446 | 2.763902 |
| SKCM | SBS17a | Unknown | 134 | 108 | 78 | 124 | 0.51938 | 0.580645 | 0.419355 | 0.48062 | 0.614901 | 0.455796 | 0.829546 |
| SKCM | SBS17b | Unknown | 183 | 114 | 72 | 75 | 0.709302 | 0.612903 | 0.387097 | 0.290698 | 1.239193 | 0.917113 | 1.674382 |
| SKCM | SBS18 | ReactiveOxy | 254 | 180 | 6 | 4 | 0.984496 | 0.967742 | 0.032258 | 0.015504 | 0.724301 | 0.316012 | 1.660103 |
| SKCM | SBS19 | Unknown | 228 | 167 | 19 | 30 | 0.883721 | 0.897849 | 0.102151 | 0.116279 | 0.75981 | 0.468753 | 1.231587 |
| SKCM | SBS21 | Mismatchrepair | 208 | 150 | 36 | 50 | 0.806202 | 0.806452 | 0.193548 | 0.193798 | 0.779914 | 0.535877 | 1.135086 |
| SKCM | SBS22 | Aristolochic acid | 203 | 144 | 42 | 55 | 0.786822 | 0.774194 | 0.225806 | 0.213178 | 1.11454 | 0.78552 | 1.581373 |
| SKCM | SBS23 | Unknown | 200 | 140 | 46 | 58 | 0.775194 | 0.752688 | 0.247312 | 0.224806 | 1.140802 | 0.815032 | 1.596784 |
| SKCM | SBS24 | Aflatoxin | 218 | 161 | 25 | 40 | 0.844961 | 0.865591 | 0.134409 | 0.155039 | 1.191448 | 0.771528 | 1.839919 |
| SKCM | SBS25 | Chemotherapy | 258 | 185 | 1 | 0 | 1 | 0.994624 | 0.005376 | 0 | 13.37649 | 1.770312 | 101.0728 |
| SKCM | SBS26 | Mismatchrepair | 211 | 148 | 38 | 47 | 0.817829 | 0.795699 | 0.204301 | 0.182171 | 0.868621 | 0.60511 | 1.246885 |
| SKCM | SBS28 | Unknown | 251 | 183 | 3 | 7 | 0.972868 | 0.983871 | 0.016129 | 0.027132 | 0.891201 | 0.283173 | 2.804781 |
| SKCM | SBS29 | Tobacco chewing in Hollst | 254 | 184 | 2 | 4 | 0.984496 | 0.989247 | 0.010753 | 0.015504 | 0.578551 | 0.129795 | 2.578855 |
| SKCM | SBS20 | Mismatchrepair + POLD1mut | 224 | 169 | 17 | 34 | 0.868217 | 0.908602 | 0.091398 | 0.131783 | 0.826792 | 0.497866 | 1.37303 |
| SKCM | SBS30 | BER repair-NTHL1mut | 206 | 139 | 47 | 52 | 0.79845 | 0.747312 | 0.252688 | 0.20155 | 1.262853 | 0.901787 | 1.768484 |
| SKCM | SBS31 | PLChemotherapy | 235 | 161 | 25 | 23 | 0.910853 | 0.865591 | 0.134409 | 0.089147 | 1.701139 | 1.105879 | 2.61681 |
| SKCM | SBS32 | AZA Chemotherapy | 250 | 176 | 10 | 8 | 0.968992 | 0.946237 | 0.053763 | 0.031008 | 1.511448 | 0.792042 | 2.884286 |
| SKCM | SBS33 | Unknown | 218 | 165 | 21 | 40 | 0.844961 | 0.887097 | 0.112903 | 0.155039 | 0.812415 | 0.511906 | 1.289335 |
| SKCM | SBS34 | Unknown | 249 | 172 | 14 | 9 | 0.965116 | 0.924731 | 0.075269 | 0.034884 | 1.940127 | 1.109363 | 3.393022 |
| SKCM | SBS35 | PLChemotherapy | 219 | 157 | 29 | 39 | 0.848837 | 0.844086 | 0.155914 | 0.151163 | 1.205954 | 0.800898 | 1.815867 |
| SKCM | SBS36 | BERrepair MUTY | 250 | 180 | 6 | 8 | 0.968992 | 0.967742 | 0.032258 | 0.031008 | 0.937069 | 0.412246 | 2.130036 |
| SKCM | SBS37 | Unknown | 250 | 177 | 9 | 8 | 0.968992 | 0.951613 | 0.048387 | 0.031008 | 1.366084 | 0.691927 | 2.697083 |
| SKCM | SBS38 | UV | 227 | 171 | 15 | 31 | 0.879845 | 0.919355 | 0.080645 | 0.120155 | 0.676967 | 0.393365 | 1.165036 |
| SKCM | SBS39 | Unknown | 212 | 159 | 27 | 46 | 0.821705 | 0.854839 | 0.145161 | 0.178295 | 0.566338 | 0.372614 | 0.860779 |
| SKCM | SBS42 | Haloalkane | 221 | 157 | 29 | 37 | 0.856589 | 0.844086 | 0.155914 | 0.143411 | 1.191907 | 0.796057 | 1.784601 |
| SKCM | SBS84 | Actind cytidinedeaminase | 238 | 168 | 18 | 20 | 0.922481 | 0.903226 | 0.096774 | 0.077519 | 1.174978 | 0.717141 | 1.925107 |
| SKCM | SBS85 | Actind cytidinedeaminase | 253 | 181 | 5 | 5 | 0.98062 | 0.973118 | 0.026882 | 0.01938 | 1.574025 | 0.640147 | 3.870287 |
| SKCM | SBS40 | Unknown | | | | | | | | | | | |
| SKCM | SBS40 | Unknown | | | | | | | | | | | |
| SKCM | SBS41 | Unknown | | | | | | | | | | | |
| SKCM | SBS44 | Mismatchrepair | | | | | | | | | | | |
| STAD | SBS1 | Endogenous clock-like age | 158 | 73 | 25 | 121 | 0.566308 | 0.744898 | 0.255102 | 0.433692 | 0.531045 | 0.333366 | 0.845943 |
| STAD | SBS2 | Endogenous Apobec3a | 236 | 88 | 10 | 43 | 0.845878 | 0.897959 | 0.102041 | 0.154122 | 0.516728 | 0.263919 | 1.011703 |
| STAD | SBS3 | Homologous repair | 268 | 91 | 7 | 11 | 0.960573 | 0.928571 | 0.071429 | 0.039427 | 1.311819 | 0.600535 | 2.865561 |
| STAD | SBS4 | Tobacco | 232 | 85 | 13 | 47 | 0.831541 | 0.867347 | 0.132653 | 0.168459 | 0.672619 | 0.371116 | 1.219069 |
| STAD | SBS6 | Mismatchrepair | 192 | 82 | 16 | 87 | 0.688172 | 0.836735 | 0.163265 | 0.311828 | 0.45336 | 0.262001 | 0.784482 |
| STAD | SBS7a | UV | 195 | 51 | 47 | 84 | 0.698925 | 0.520408 | 0.479592 | 0.301075 | 1.690301 | 1.124845 | 2.540011 |
| STAD | SBS7b | UV | 169 | 71 | 27 | 110 | 0.605735 | 0.72449 | 0.27551 | 0.394265 | 0.569586 | 0.360672 | 0.899511 |
| STAD | SBS7c | UV | 245 | 81 | 17 | 34 | 0.878136 | 0.826531 | 0.173469 | 0.121864 | 1.339128 | 0.788672 | 2.273778 |
| STAD | SBS7d | UV | 245 | 81 | 17 | 34 | 0.878136 | 0.826531 | 0.173469 | 0.121864 | 1.763232 | 1.039056 | 2.992126 |
| STAD | SBS8 | Unknown | 221 | 67 | 31 | 58 | 0.792115 | 0.683673 | 0.316327 | 0.207885 | 1.64977 | 1.056313 | 2.576643 |
| STAD | SBS9 | PolETA Somatichypermutation | 270 | 95 | 3 | 9 | 0.967742 | 0.969388 | 0.030612 | 0.032258 | 0.902313 | 0.277407 | 2.93492 |
| STAD | SBS10a | Polymerase epsilon hyperm [symbol] | 216 | 69 | 29 | 63 | 0.774194 | 0.704082 | 0.295918 | 0.225806 | 1.449412 | 0.926367 | 2.26778 |
| STAD | SBS10b | Polymerase epsilon mutati [symbol] | 196 | 86 | 12 | 83 | 0.702509 | 0.877551 | 0.122449 | 0.297491 | 0.358713 | 0.194138 | 0.662801 |
| STAD | SBS11 | Temozolo Chemotherapy | 262 | 88 | 10 | 17 | 0.939068 | 0.897959 | 0.102041 | 0.060932 | 1.696239 | 0.873695 | 3.293173 |
| STAD | SBS12 | Liver cancer | 235 | 75 | 23 | 44 | 0.842294 | 0.765306 | 0.234694 | 0.157706 | 1.62552 | 1.007189 | 2.623457 |
| STAD | SBS13 | Endogenous apobec | 198 | 76 | 22 | 81 | 0.709677 | 0.77551 | 0.22449 | 0.290323 | 0.651343 | 0.399501 | 1.061945 |
| STAD | SBS14 | Mismatchrepair + PolEpsil | 236 | 86 | 12 | 43 | 0.845878 | 0.877551 | 0.122449 | 0.154122 | 0.6576 | 0.352705 | 1.22606 |
| STAD | SBS15 | Mismatchrepair | 240 | 92 | 6 | 39 | 0.860215 | 0.938776 | 0.061224 | 0.139785 | 0.390017 | 0.169171 | 0.899166 |
| STAD | SBS16 | Unknown | 237 | 93 | 5 | 42 | 0.849462 | 0.94898 | 0.05102 | 0.150538 | 0.348821 | 0.140857 | 0.863826 |
| STAD | SBS17 | Unknown | 204 | 64 | 34 | 75 | 0.731183 | 0.653061 | 0.346939 | 0.268817 | 1.142538 | 0.740985 | 1.761702 |
| STAD | SBS17 | Unknown | 237 | 79 | 19 | 42 | 0.849462 | 0.806122 | 0.193878 | 0.150538 | 1.33957 | 0.799462 | 2.244568 |
| STAD | SBS18 | ReactiveOxy | 193 | 66 | 32 | 86 | 0.691756 | 0.673469 | 0.326531 | 0.308244 | 1.158349 | 0.751285 | 1.78597 |
| STAD | SBS19 | Unknown | 238 | 86 | 12 | 41 | 0.853047 | 0.877551 | 0.122449 | 0.146953 | 0.826889 | 0.44238 | 1.545604 |
| STAD | SBS21 | Mismatchrepair | 241 | 90 | 8 | 38 | 0.863799 | 0.918367 | 0.081633 | 0.136201 | 0.473979 | 0.22867 | 0.982446 |
| STAD | SBS22 | Aristolochic acid | 183 | 59 | 39 | 96 | 0.655914 | 0.602041 | 0.397959 | 0.344086 | 1.478127 | 0.979722 | 2.230083 |
| STAD | SBS23 | Unknown | 262 | 96 | 2 | 17 | 0.939068 | 0.979592 | 0.020408 | 0.060932 | 0.520296 | 0.127169 | 2.128723 |
| STAD | SBS24 | Aflatoxin | 251 | 87 | 11 | 28 | 0.899642 | 0.887755 | 0.112245 | 0.100358 | 1.214573 | 0.638974 | 2.308684 |
| STAD | SBS25 | Chemotherapy | 246 | 90 | 8 | 33 | 0.88172 | 0.918367 | 0.081633 | 0.11828 | 0.718158 | 0.342922 | 1.50399 |
| STAD | SBS26 | Mismatchrepair | 226 | 86 | 12 | 53 | 0.810036 | 0.877551 | 0.122449 | 0.189964 | 0.516085 | 0.277004 | 0.961514 |
| STAD | SBS28 | Unknown | 247 | 89 | 9 | 32 | 0.885305 | 0.908163 | 0.091837 | 0.114695 | 0.735689 | 0.36679 | 1.475605 |
| STAD | SBS29 | Tobacco chewing in Hollst | 169 | 74 | 24 | 110 | 0.605735 | 0.755102 | 0.244898 | 0.394265 | 0.552913 | 0.346481 | 0.882335 |
| STAD | SBS20 | Mismatchrepair + POLD1mut | 219 | 82 | 16 | 60 | 0.784946 | 0.836735 | 0.163265 | 0.215054 | 0.60074 | 0.342842 | 1.052637 |
| STAD | SBS30 | BER repair-NTHL1mut | 245 | 83 | 15 | 34 | 0.878136 | 0.846939 | 0.153061 | 0.121864 | 1.129391 | 0.645952 | 1.974644 |
| STAD | SBS31 | PLChemotherapy | 213 | 80 | 18 | 66 | 0.763441 | 0.816327 | 0.183673 | 0.236559 | 0.723888 | 0.422355 | 1.240695 |
| STAD | SBS32 | AZA Chemotherapy | 247 | 90 | 8 | 32 | 0.885305 | 0.918367 | 0.081633 | 0.114695 | 0.717479 | 0.345356 | 1.490566 |
| STAD | SBS33 | Unknown | 170 | 68 | 30 | 109 | 0.609319 | 0.693878 | 0.306122 | 0.390681 | 0.599262 | 0.385692 | 0.931092 |
| STAD | SBS34 | Unknown | 232 | 86 | 12 | 47 | 0.831541 | 0.877551 | 0.122449 | 0.168459 | 0.678022 | 0.368468 | 1.247635 |
| STAD | SBS35 | PLChemotherapy | 221 | 84 | 14 | 58 | 0.792115 | 0.857143 | 0.142857 | 0.207885 | 0.712483 | 0.401196 | 1.265296 |
| STAD | SBS36 | BERrepair MUTY | 244 | 82 | 16 | 35 | 0.874552 | 0.836735 | 0.163265 | 0.125448 | 1.190653 | 0.68153 | 2.080106 |
| STAD | SBS37 | Unknown | 236 | 84 | 14 | 43 | 0.845878 | 0.857143 | 0.142857 | 0.154122 | 0.829077 | 0.46712 | 1.471502 |
| STAD | SBS38 | UV | 221 | 74 | 24 | 58 | 0.792115 | 0.755102 | 0.244898 | 0.207885 | 1.27652 | 0.79526 | 2.049019 |
| STAD | SBS39 | Unknown | 103 | 30 | 68 | 176 | 0.369176 | 0.306122 | 0.693878 | 0.630824 | 1.37563 | 0.88816 | 2.13065 |
| STAD | SBS42 | Haloalkane | 269 | 94 | 4 | 10 | 0.964158 | 0.959184 | 0.040816 | 0.035842 | 1.126325 | 0.409118 | 3.100832 |
| STAD | SBS84 | Actind cytidinedeaminase | 255 | 90 | 8 | 24 | 0.913978 | 0.918367 | 0.081633 | 0.086022 | 1.134929 | 0.541858 | 2.377122 |
| STAD | SBS85 | Actind cytidinedeaminase | 255 | 88 | 10 | 24 | 0.913978 | 0.897959 | 0.102041 | 0.086022 | 1.264047 | 0.653662 | 2.444405 |
| STAD | SBS40 | Unknown | | | | | | | | | | | |
| STAD | SBS41 | Unknown | | | | | | | | | | | |
| STAD | SBS44 | Mismatchrepair | | | | | | | | | | | |
| UCEC | SBS1 | Endogenous clock-like age | 230 | 43 | 14 | 234 | 0.49569 | 0.754386 | 0.245614 | 0.50431 | 0.303877 | 0.16276 | 0.567346 |
| UCEC | SBS2 | Endogenous Apobec3a | 304 | 30 | 27 | 160 | 0.655172 | 0.526316 | 0.473684 | 0.344828 | 1.500275 | 0.871621 | 2.582342 |
| UCEC | SBS3 | Homologous repair | 450 | 54 | 3 | 14 | 0.969828 | 0.947368 | 0.052632 | 0.030172 | 1.468458 | 0.452606 | 4.764342 |
| UCEC | SBS4 | Tobacco | 378 | 49 | 8 | 86 | 0.814655 | 0.859649 | 0.140351 | 0.185345 | 0.807646 | 0.381574 | 1.709479 |
| UCEC | SBS6 | Mismatchrepair | 395 | 56 | 1 | 69 | 0.851293 | 0.982456 | 0.017544 | 0.148707 | 0.064567 | 0.008904 | 0.468204 |
| UCEC | SBS7a | UV | 302 | 34 | 23 | 162 | 0.650862 | 0.596491 | 0.403509 | 0.349138 | 1.231566 | 0.715176 | 2.120812 |
| UCEC | SBS7c | UV | 403 | 54 | 3 | 61 | 0.868534 | 0.947368 | 0.052632 | 0.131466 | 0.306673 | 0.095188 | 0.988031 |
| UCEC | SBS7d | UV | 392 | 50 | 7 | 72 | 0.844828 | 0.877193 | 0.122807 | 0.155172 | 0.877847 | 0.395833 | 1.946819 |
| UCEC | SBS8 | Unknown | 390 | 43 | 14 | 74 | 0.840517 | 0.754386 | 0.245614 | 0.159483 | 1.750593 | 0.941599 | 3.254649 |
| UCEC | SBS9 | PolETA Somatichypermutation | 443 | 57 | 0 | 21 | 0.954741 | 1 | 0 | 0.045259 | 1.08E−06 | 0 | |
| UCEC | SBS10 | Polymerase epsilon hyperm [symbol] | 399 | 56 | 1 | 65 | 0.859914 | 0.982456 | 0.017544 | 0.140086 | 0.063907 | 0.008739 | 0.467363 |
| UCEC | SBS10 | Polymerase epsilon mutati [symbol] | 386 | 55 | 2 | 78 | 0.831897 | 0.964912 | 0.035088 | 0.168103 | 0.130551 | 0.031422 | 0.542416 |
| UCEC | SBS11 | Temozolo Chemotherapy | 400 | 52 | 5 | 64 | 0.862069 | 0.912281 | 0.087719 | 0.137931 | 0.511451 | 0.204055 | 1.281925 |
| UCEC | SBS12 | Liver cancer | 397 | 45 | 12 | 67 | 0.855603 | 0.789474 | 0.210526 | 0.144397 | 1.546494 | 0.81199 | 2.94541 |
| UCEC | SBS13 | Endogenous apobec | 322 | 29 | 28 | 142 | 0.693966 | 0.508772 | 0.491228 | 0.306034 | 1.761385 | 1.024621 | 3.027926 |
| UCEC | SBS14 | Mismatchrepair + PolEpsil | 371 | 54 | 3 | 93 | 0.799569 | 0.947368 | 0.052632 | 0.200431 | 0.128418 | 0.039269 | 0.419955 |
| UCEC | SBS15 | Mismatchrepair | 370 | 53 | 4 | 94 | 0.797414 | 0.929825 | 0.070175 | 0.202586 | 0.225888 | 0.080229 | 0.636001 |
| UCEC | SBS16 | Unknown | 325 | 30 | 27 | 139 | 0.700431 | 0.526316 | 0.473684 | 0.299569 | 2.174137 | 1.278438 | 3.697381 |
| UCEC | SBS17a | Unknown | 313 | 50 | 7 | 151 | 0.674569 | 0.877193 | 0.122807 | 0.325431 | 0.285819 | 0.127618 | 0.640131 |
| UCEC | SBS17b | Unknown | 397 | 54 | 3 | 67 | 0.855603 | 0.947368 | 0.052632 | 0.144397 | 0.218838 | 0.067359 | 0.71097 |
| UCEC | SBS18 | ReactiveOxy | 398 | 43 | 14 | 66 | 0.857759 | 0.754386 | 0.245614 | 0.142241 | 1.98249 | 1.083737 | 3.626587 |
| UCEC | SBS19 | Unknown | 355 | 49 | 8 | 109 | 0.765086 | 0.859649 | 0.140351 | 0.234914 | 0.717542 | 0.337949 | 1.523505 |
| UCEC | SBS21 | Mismatchrepair | 366 | 48 | 9 | 98 | 0.788793 | 0.842105 | 0.157895 | 0.211207 | 0.492341 | 0.237949 | 1.018704 |
| UCEC | SBS22 | Aristolochic acid | 409 | 46 | 11 | 55 | 0.881466 | 0.807018 | 0.192982 | 0.118534 | 1.973855 | 1.021067 | 3.815715 |
| UCEC | SBS23 | Unknown | 411 | 48 | 9 | 53 | 0.885776 | 0.842105 | 0.157895 | 0.114224 | 1.078653 | 0.528435 | 2.20177 |
| UCEC | SBS24 | Aflatoxin | 332 | 34 | 23 | 132 | 0.715517 | 0.596491 | 0.403509 | 0.284483 | 1.635475 | 0.949361 | 2.817451 |
| UCEC | SBS25 | Chemotherapy | 437 | 55 | 2 | 27 | 0.94181 | 0.964912 | 0.035088 | 0.05819 | 0.85212 | 0.206278 | 3.520058 |
| UCEC | SBS26 | Mismatchrepair | 298 | 46 | 11 | 166 | 0.642241 | 0.807018 | 0.192982 | 0.357759 | 0.424395 | 0.214953 | 0.83791 |
| UCEC | SBS28 | Unknown | 373 | 52 | 5 | 91 | 0.803879 | 0.912281 | 0.087719 | 0.196121 | 0.43521 | 0.172525 | 1.097854 |
| UCEC | SBS29 | Tobacco chewing in Hollst | 301 | 31 | 26 | 163 | 0.648707 | 0.54386 | 0.45614 | 0.351293 | 1.822548 | 1.066883 | 3.113447 |
| UCEC | SBS20 | Mismatchrepair + POLD1mut | 403 | 55 | 2 | 61 | 0.868534 | 0.964912 | 0.035088 | 0.131466 | 0.17879 | 0.043454 | 0.735631 |
| UCEC | SBS30 | BER repair-NTHL1mut | 419 | 46 | 11 | 45 | 0.903017 | 0.807018 | 0.192982 | 0.096983 | 1.927281 | 0.995245 | 3.732161 |
| UCEC | SBS31 | PLChemotherapy | 394 | 49 | 8 | 70 | 0.849138 | 0.859649 | 0.140351 | 0.150862 | 0.828878 | 0.391941 | 1.752913 |
| UCEC | SBS32 | AZA Chemotherapy | 412 | 53 | 4 | 52 | 0.887931 | 0.929825 | 0.070175 | 0.112069 | 0.490073 | 0.175761 | 1.366465 |
| UCEC | SBS33 | Unknown | 300 | 51 | 6 | 164 | 0.646552 | 0.894737 | 0.105263 | 0.353448 | 0.150823 | 0.062906 | 0.36161 |
| UCEC | SBS34 | Unknown | 354 | 36 | 21 | 110 | 0.762931 | 0.631579 | 0.368421 | 0.237069 | 1.938963 | 1.130088 | 3.326801 |
| UCEC | SBS35 | PLChemotherapy | 402 | 50 | 7 | 62 | 0.866379 | 0.877193 | 0.122807 | 0.133621 | 0.973323 | 0.440499 | 2.150646 |
| UCEC | SBS36 | BERrepair MUTY | 402 | 50 | 7 | 62 | 0.866379 | 0.877193 | 0.122807 | 0.133621 | 0.931541 | 0.422134 | 2.055672 |
| UCEC | SBS37 | Unknown | 412 | 54 | 3 | 52 | 0.887931 | 0.947368 | 0.052632 | 0.112069 | 0.427947 | 0.132367 | 1.38357 |
| UCEC | SBS38 | UV | 399 | 55 | 2 | 65 | 0.859914 | 0.964912 | 0.035088 | 0.140086 | 0.204082 | 0.049504 | 0.841339 |
| UCEC | SBS39 | Unknown | 284 | 19 | 38 | 180 | 0.612069 | 0.333333 | 0.666667 | 0.387931 | 2.710644 | 1.540951 | 4.768219 |
| UCEC | SBS42 | Haloalkane | 390 | 42 | 15 | 74 | 0.840517 | 0.736842 | 0.263158 | 0.159483 | 1.44319 | 0.799695 | 2.60449 |
| UCEC | SBS84 | Actind cytidinedeaminase | 417 | 49 | 8 | 47 | 0.898707 | 0.859649 | 0.140351 | 0.101293 | 1.174637 | 0.555513 | 2.483782 |
| UCEC | SBS85 | Actind cytidinedeaminase | 435 | 54 | 3 | 29 | 0.9375 | 0.947368 | 0.052632 | 0.0625 | 0.714982 | 0.223157 | 2.290762 |
| UCEC | SBS40 | Unknown | | | | | | | | | | | |
| UCEC | SBS41 | Unknown | | | | | | | | | | | |
| UCEC | SBS44 | Mismatchrepair | | | | | | | | | | | |
| UCEC | SBS7b | UV | | | | | | | | | | | |

[symbol]

indicates data missing or illegible when filed

### Abbreviations

BER—Base Excision Repair
AID—Activation-induced cytidine deaminase
*All hazard ratios adjusted for diagnosis age, sex (except gender-specific cancers), and cancer stage. The latter was omitted when the signature was associated with stage, as stage was then considered to potentially be on the causal pathway between exposure (signature) and outcome. Immortal person-time adjustments were incorporated.

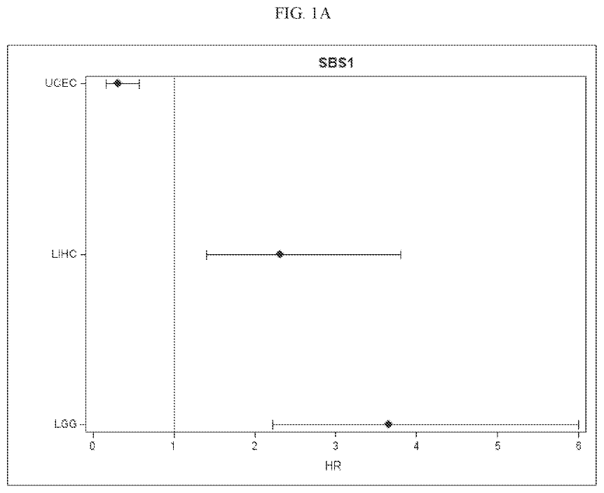

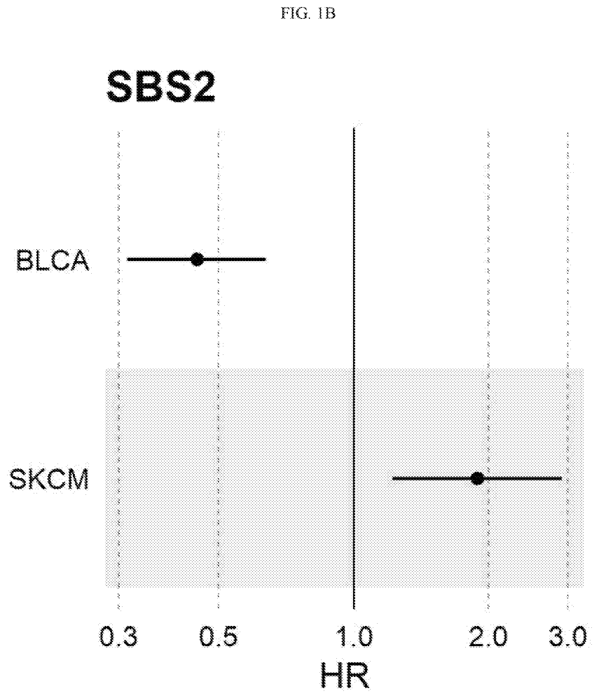

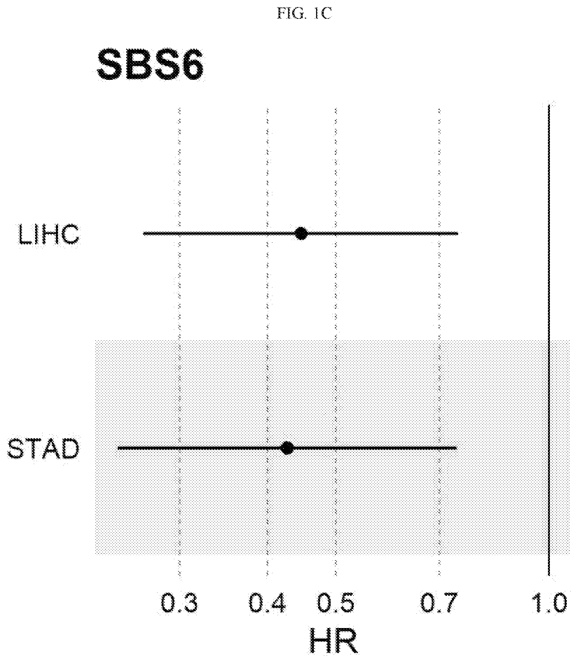

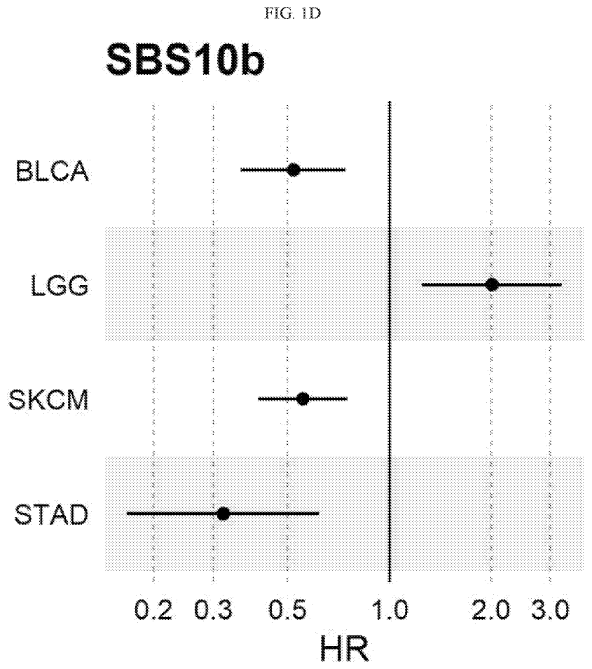

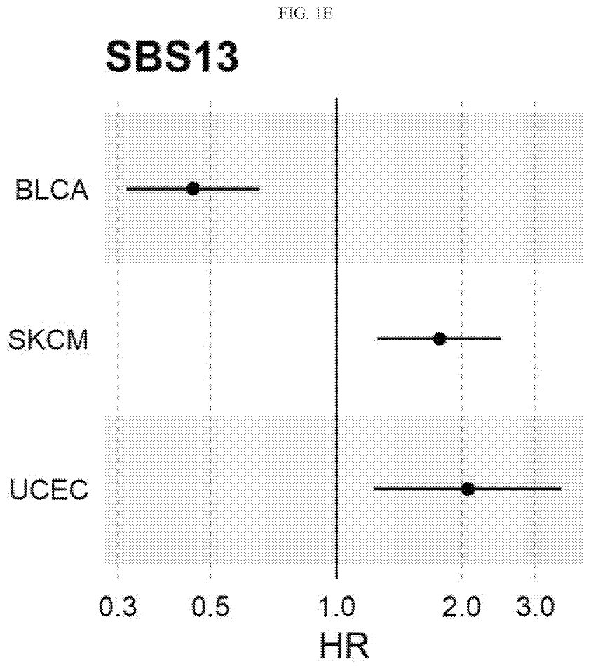

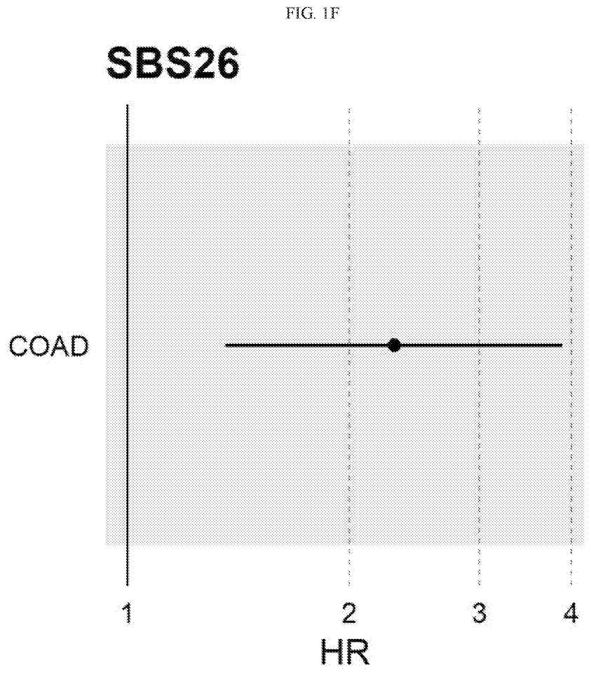

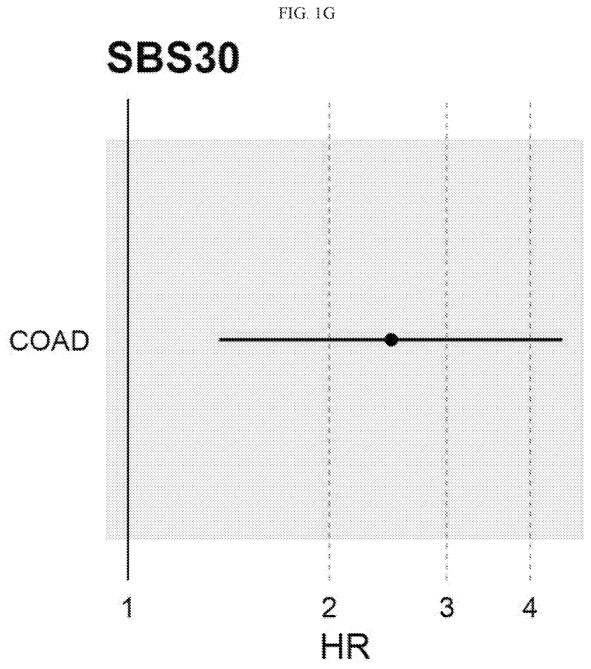

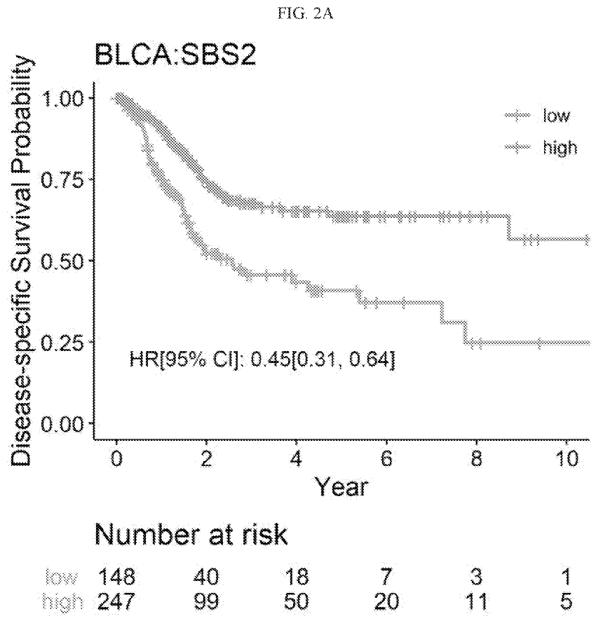

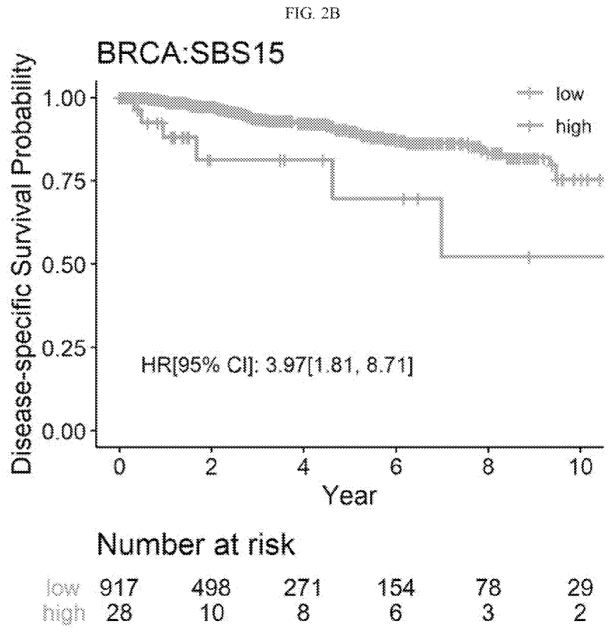

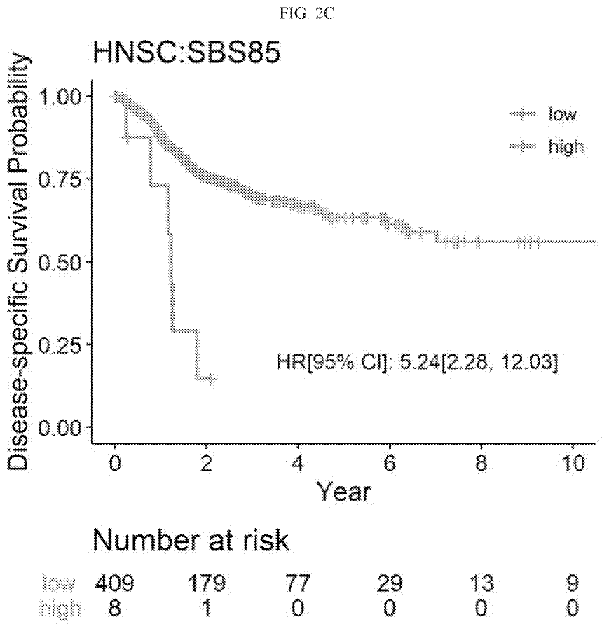

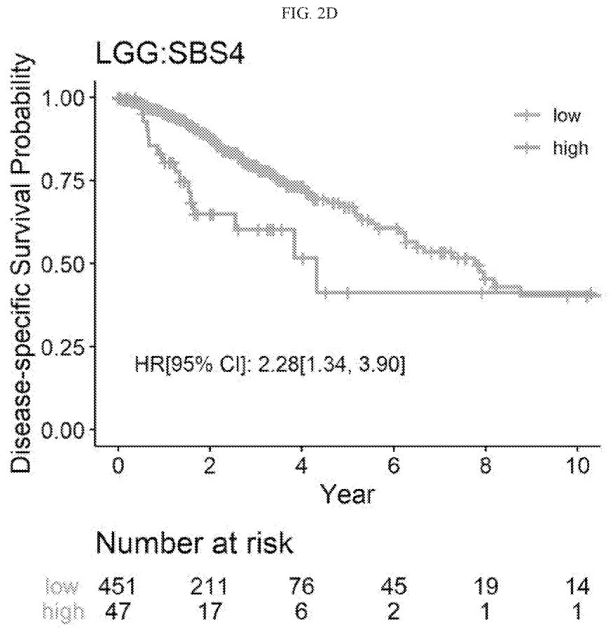

Among endogenous mutation measures (FIGS. 1 and 2), the most common etiology of the mutational signatures identified was a clock-like signature associated with aging (SBS1), implicated in survival of 6 cancers, including Colon adenocarcinoma (COAD), LGG, Liver hepatocellular carcinoma (LIHC), OV, Stomach adenocarcinoma (STAD), and Uterine Corpus Endometrial Carcinoma (UCEC) (Table 2 and FIG. 1). The effect of the aging signature on survival was mixed, in that risk of cancer-specific mortality was reduced among participants with COAD, STAD and UCEC cancers, but elevated up to 5.5-fold in other cancers. Two signatures (SBS2 and SBS13) of deregulated APOBEC-related cytosine deamination activity were both associated with reduced mortality in BLCA patients, but in those with LGG, LIHC, and Skin Cutaneous Melanoma (SKCM) cancers, risk of disease-specific mortality was elevated up to 6-fold. Defects in DNA repair mechanisms that may have contributed to altered survival include several signatures of mismatch repair (SBS6, 15, 20, 26) in BRCA, COAD, LIHC, and STAD tumors, in which LIHC and STAD patients with SBS6 experienced a reduced mortality risk, but risks were elevated otherwise. A base excision repair signature (SBS30) was related to 2.5 and 6-fold increased mortality, respectively, in COAD and LGG. Polymerase epsilon, which acts in both replication and DNA recombination, also facilitates completion of base excision and nucleotide excision repair, thus signatures of defective polymerase epsilon (SBS10a, SBS10b) in BLCA, BRCA, LGG, SKCM, or STAD survival could be attributed to these several cellular mechanisms. In addition to the above, a further endogenous process implicated in DSS is activation-induced cytidine deaminase in 3.5 and 5-fold increased risks of disease-specific mortality in LIHC and Head and Neck squamous cell carcinoma (HNSC).

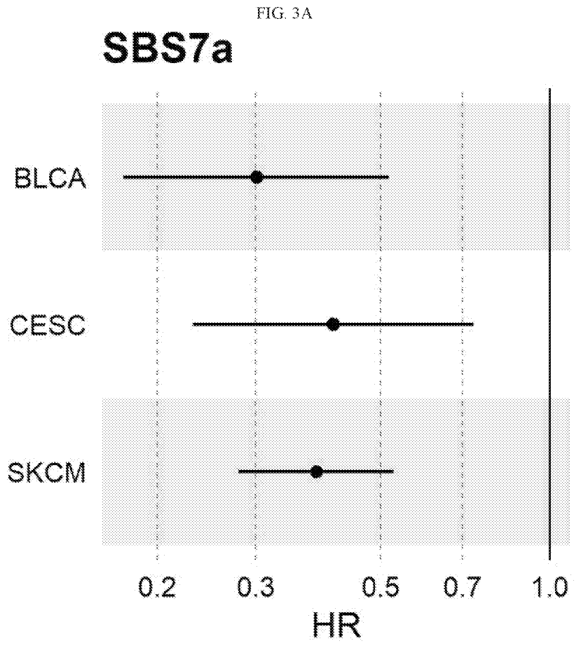

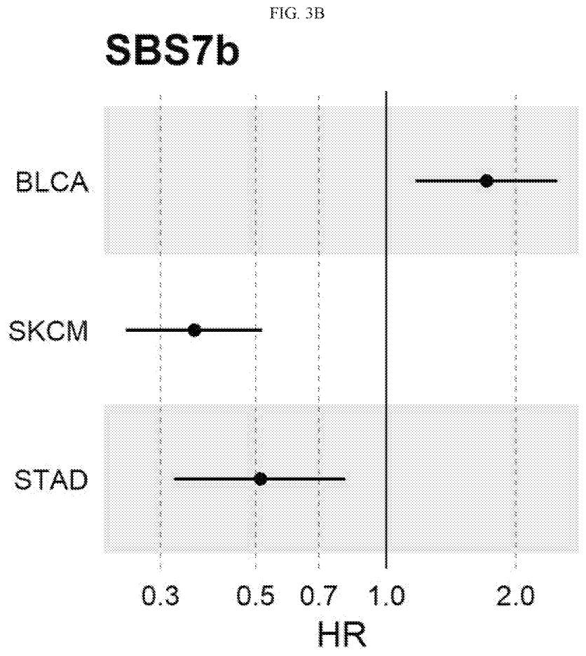

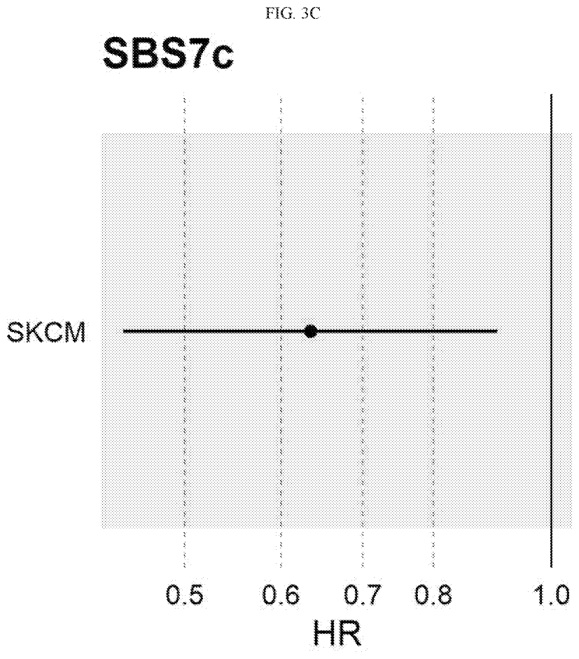

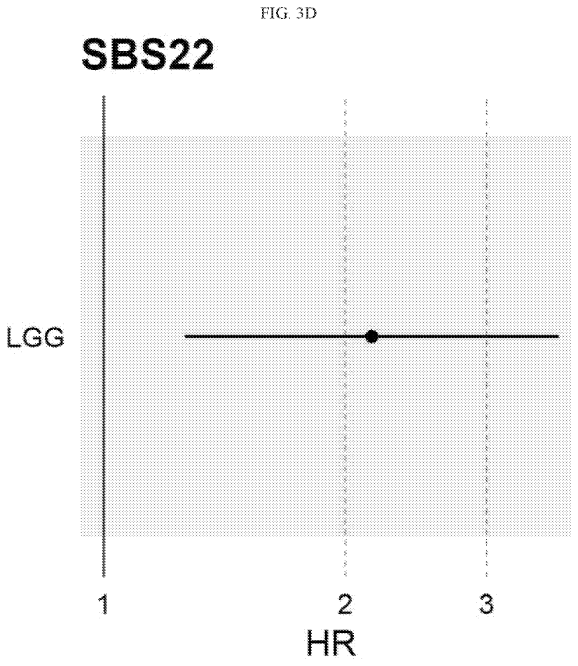

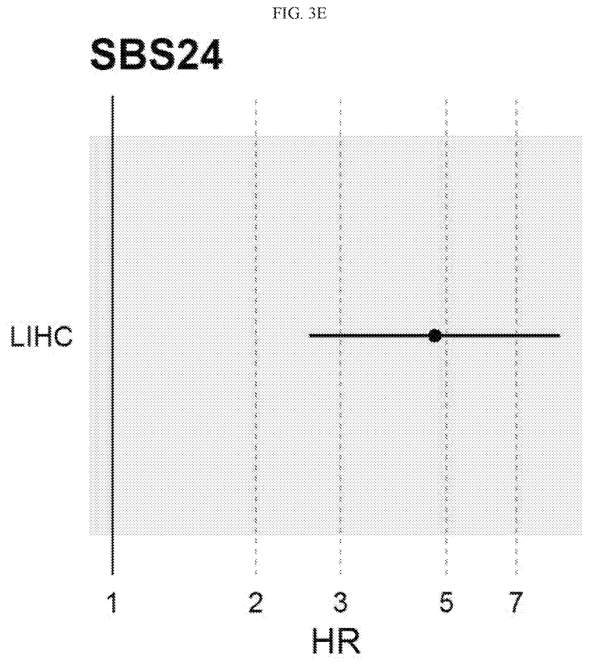

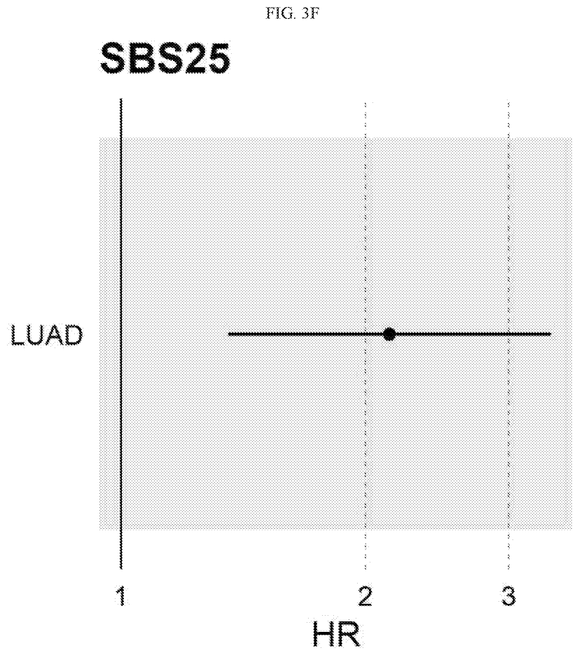

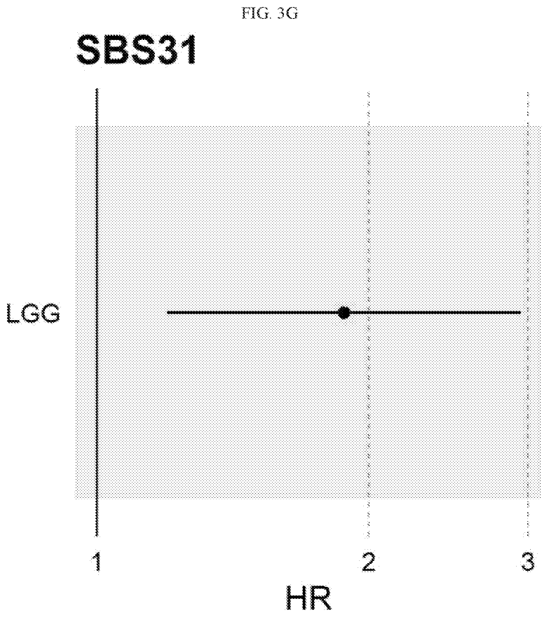

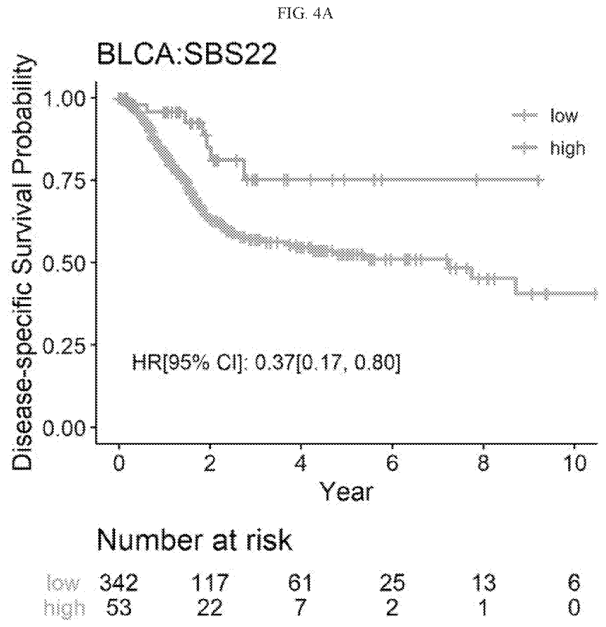

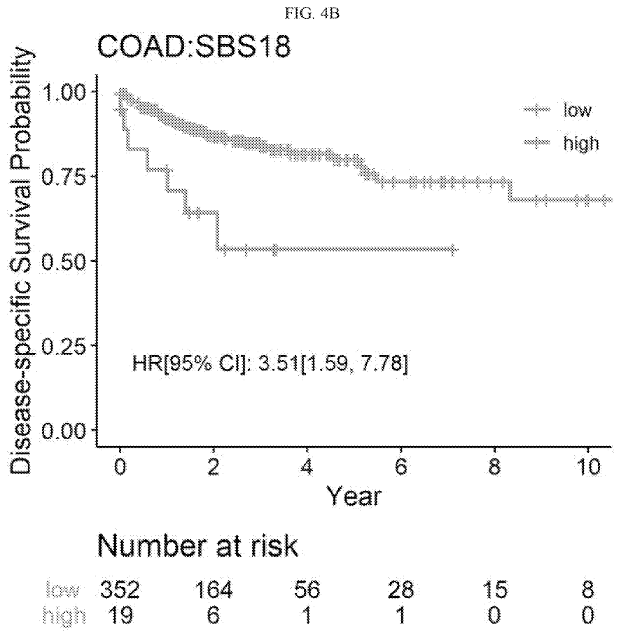

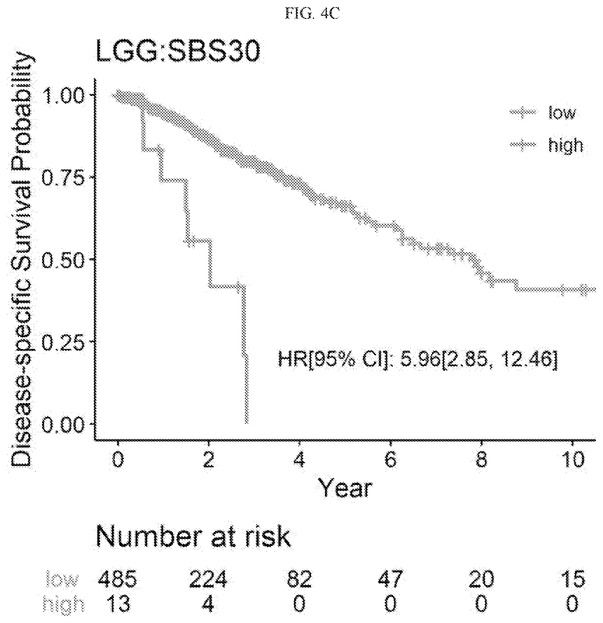

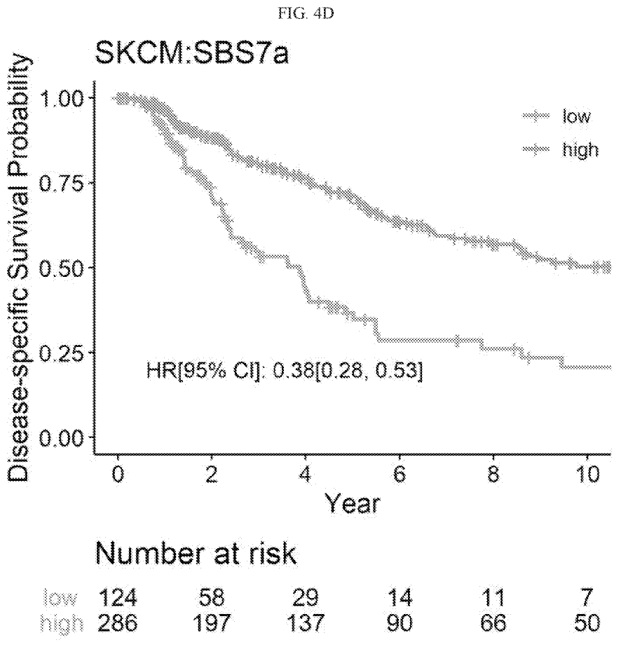

Among signatures of exogenous mutagenic agents (FIGS. 3 and 4), that associated with aristolochic acid, an herbal medicine component (SBS22), was related to reduced mortality in BLCA, but to 2 to 3.8-fold increased mortality in COAD, LGG, and SARC (Table 2). Ultraviolet (UV) exposure, captured by the SBS7a, 7b, 7c, and SBS38 signatures, differed in relationship to mortality by type of cancer and signature, with BLCA patients experiencing both reduced and increased risks, LIHC patients only increased risks, while all risks were reduced in SKCM patients. Those with signatures of tobacco exposure (Hollstein et al., 2017) (SBS4, SBS29) had increased risks of cancer mortality in BRCA (4-fold), LGG (2.2 to 2.8-fold), and SKCM (3-fold). COAD, LGG, LUSC, and SKCM patients bearing a signature of previous chemotherapy treatment (SBS11, SBS25, SBS31) had 4 to 5-fold elevated risks of mortality due to their respective cancers, while chemotherapy-related mortality was elevated 2.5-fold in BLCA. CESC and COAD-specific mortality were increased 3-8-fold among those whose cancers demonstrated the mutational signatures of haloalkanes (SBS42), an organic solvent exposure incurred occupationally (Mimaki et al., 2016). Aflatoxin mutagenesis (SBS24) was associated with a 3-fold increased risk of LGG-specific mortality and a greater than 13-fold increased risk of mortality among those with LIHC. Among COAD patients, those with a signature of reactive oxygen species (SBS18), considered a metabolite of carcinogenic exposures but also generated endogenously, had a 3.5-fold increased risk of disease-specific mortality.

To evaluate one possible mechanism whereby mutational signatures might influence survival, the relationship between signature and stage within each cancer (Table 2) was assessed. Of the 46 evaluable relationships, 10 were also associated with stage at diagnosis when assessed using continuous, discrete, or binary survival cutpoint measures. However, many with strong relationships with DSS (continuous or discrete, and binary cutoff) were not (COAD and SBS26 (mismatch repair), LIHC and SBS1 (Endogenous clock-like age), SKCM and SBS7b (UV), and SKCM and SBS10b (Polymerase Epsilon) are among those).

TABLE 3A
Relative risk for diagnosis with stage 3 or 4 disease, relative to stage 1,
according to mutational signatures related to disease-specific survival.

| | | | | | Maxstat | | |
|---|---|---|---|---|---|---|---|
| | | | Continuous | Discrete | Relative | | |
| Cancer* | Signature | Signature Description | (p-value**) | (p-value**) | Risk*** | Lower CI | Upper CI |
| BRCA | SBS12 | Liver-cancer related | 0.0325 | 0.61 | 1.3438 | 1.0396 | 1.7368 |
| | SBS22 | Aristolochic acid | 0.0213 | 0.14 | 0.6237 | 0.4321 | 0.9004 |
| CESC | _all_{—} | Stage missing | | | | | |
| COAD | SBS1 | Endogenous clock-like age | 0.016 | 0.0155 | 0.6250 | 0.4866 | 0.8026 |
| | SBS6 | Mismatch Repair | 0.0055 | 0.0075 | 0.6076 | 0.4438 | 0.8318 |
| | SBS14 | Mismatchrepair + PolEpsilon | 0.0063 | 0.0021 | 0.4786 | 0.2924 | 0.7836 |
| | SBS15 | Mismatch repair | 0.0034 | 0.0185 | 0.4181 | 0.237 | 0.7376 |
| | SBS20 | Mismatchrepair + POLD1mut | 0.0021 | 0.0041 | 0.6123 | 0.4247 | 0.8826 |
| | SBS33 | Unknown | 0.0012 | 0.0018 | 0.7148 | 0.5332 | 0.9583 |
| | SBS39 | Unknown | 0.2244 | 0.9573 | 1.3751 | 1.038 | 1.8218 |
| HNSC | SBS2 | Endogenous Apobec3a | 0.1445 | 0.3949 | 1.1640 | 1.0163 | 1.3332 |
| | SBS4 | Tobacco | 0.1959 | 0.732 | 1.1444 | 1.0195 | 1.2845 |
| | SBS24 | Aflatoxin | 0.2453 | 0.375 | 1.1306 | 1.0242 | 1.248 |
| | SBS29 | Tobacco | 0.1974 | 0.2593 | 1.1500 | 1.0409 | 1.2705 |
| | SBS31 | PLChemotherapy | 0.2613 | 0.5023 | 1.1273 | 1.0007 | 1.2699 |
| LGG | _all_{—} | Stage missing | | | | | |
| LIHC | SBS24 | Aflatoxin | 0.0715 | 0.0474 | 1.7451 | 1.122 | 2.714 |
| | SBS25 | Chemotherapy | 0.7238 | 0.793 | 1.5335 | 1.0309 | 2.2814 |
| | SBS84 | Actind cytidine deaminase | 0.0088 | 0.2167 | 1.7365 | 1.1284 | 2.6724 |
| LUAD | SBS25 | Chemotherapy | 0.3480 | 0.437 | 1.5546 | 1.0058 | 2.4030 |
| | SBS29 | Tobacco | 0.0068 | 0.0882 | 1.5384 | 1.0089 | 2.3458 |
| LUSC | SBS1 | Endogenous clock-like age | 0.2526 | 0.5205 | 3.9657 | 1.0116 | 15.5467 |
| | SBS23 | Unknown | 0.3356 | 0.5686 | 2.2129 | 1.3928 | 3.5159 |
| | SBS25 | Chemotherapy | 0.0411 | 0.0949 | 1.6952 | 1.0646 | 2.6992 |
| OV | _all_{—} | Stage missing | | | | | |
| PAAD | SBS32 | AZA Chemotherapy | 0.1111 | 0.8842 | 4.2668 | 1.2007 | 15.163 |
| SARC | _all_{—} | Stage missing | | | | | |
| SKCM | SBS4 | Tobacco | 0.0039 | 0.9926 | 1.6503 | 1.271 | 2.1429 |
| | SBS7c | Ultraviolet | 0.3486 | 0.8503 | 0.7935 | 0.6377 | 0.9874 |
| | SBS10b | Polymerase epsilon | 0.7141 | 0.6232 | 0.8091 | 0.656 | 0.998 |
| | | mutation | | | | | |
| | SBS36 | Base excision repair MUTY | 0.3444 | 0.9259 | 1.5259 | 1.0355 | 2.2485 |
| | SBS38 | Ultraviolet | 0.0334 | 0.0264 | 0.5749 | 0.3437 | 0.9618 |
| STAD | SBS1 | Endogenous clock-like age | 0.0106 | 0.7954 | 0.8651 | 0.6997 | 1.0697 |
| | SBS7c | Ultraviolet | 0.0154 | 0.7877 | 1.2383 | 0.9872 | 1.5531 |
| | SBS16 | Unknown | 0.0308 | 0.6479 | 0.6958 | 0.4827 | 1.0000 |
| | SBS19 | Unknown | 0.0072 | 0.1246 | 1.0700 | 0.8730 | 1.3115 |
| | SBS20 | Mismatchrepair + POLD1mut | 0.0845 | 0.0143 | 0.8688 | 0.6594 | 1.1447 |
| | SBS26 | Mismatchrepair | 0.0303 | 0.6375 | 0.8767 | 0.6264 | 1.2190 |
| | SBS29 | Tobacco | 0.0500 | 0.1886 | 0.9649 | 0.7883 | 1.1810 |
| | SBS34 | Unknown | 0.0089 | 0.7431 | 1.1945 | 0.9546 | 1.4946 |
| | SBS39 | Unknown | 0.0280 | 0.4674 | 1.1622 | 0.9439 | 1.4309 |
| UCEC | _all_{—} | Stage missing | | | | | |

All relationships adjusted for age and gender, except gender omitted for BRCA, CESC, OV, and UCEC.
ND—Not determined due to model non-convergence
*BLCA omitted due to only n = 2 cancers diagnosed at Stage I
**Relative risk calculated utilizing the cutpoint determined using maximally selected rank statistics

TABLE 3B
Relative risk for diagnosis with stage 3 or 4 disease, relative to stage 1 or
2, according to mutational signatures related to disease-specific survival.

| | | | | | Maxstat | | |
|---|---|---|---|---|---|---|---|
| | | | Continuous | Discrete | Relative | | |
| Cancer* | Signature | Signature Description | (p-value**) | (p-value**) | Risk*** | Lower CI | Upper CI |
| BRCA | SBS12 | Liver-cancer related | 0.0325 | 0.61 | 1.3438 | 1.0396 | 1.7368 |
| | SBS22 | Aristolochic acid | 0.0213 | 0.14 | 0.6237 | 0.4321 | 0.9004 |
| CESC | _all_{—} | Stage missing | | | | | |
| COAD | SBS1 | Endogenous clock-like age | 0.016 | 0.0155 | 0.6250 | 0.4866 | 0.8026 |
| | SBS6 | Mismatch Repair | 0.0055 | 0.0075 | 0.6076 | 0.4438 | 0.8318 |
| | SBS14 | Mismatchrepair + PolEpsilon | 0.0063 | 0.0021 | 0.4786 | 0.2924 | 0.7836 |
| | SBS15 | Mismatch repair | 0.0034 | 0.0185 | 0.4181 | 0.237 | 0.7376 |
| | SBS20 | Mismatchrepair + POLD1mut | 0.0021 | 0.0041 | 0.6123 | 0.4247 | 0.8826 |
| | SBS33 | Unknown | 0.0012 | 0.0018 | 0.7148 | 0.5332 | 0.9583 |
| | SBS39 | Unknown | 0.2244 | 0.9573 | 1.3751 | 1.038 | 1.8218 |
| HNSC | SBS2 | Endogenous Apobec3a | 0.1445 | 0.3949 | 1.1640 | 1.0163 | 1.3332 |
| | SBS4 | Tobacco | 0.1959 | 0.732 | 1.1444 | 1.0195 | 1.2845 |
| | SBS24 | Aflatoxin | 0.2453 | 0.375 | 1.1306 | 1.0242 | 1.248 |
| | SBS29 | Tobacco | 0.1974 | 0.2593 | 1.1500 | 1.0409 | 1.2705 |
| | SBS31 | PLChemotherapy | 0.2613 | 0.5023 | 1.1273 | 1.0007 | 1.2699 |
| LGG | _all_{—} | Stage missing | | | | | |
| LIHC | SBS24 | Aflatoxin | 0.0715 | 0.0474 | 1.7451 | 1.122 | 2.714 |
| | SBS25 | Chemotherapy | 0.7238 | 0.793 | 1.5335 | 1.0309 | 2.2814 |
| | SBS84 | Actind cytidine deaminase | 0.0088 | 0.2167 | 1.7365 | 1.1284 | 2.6724 |
| LUAD | SBS25 | Chemotherapy | 0.3480 | 0.437 | 1.5546 | 1.0058 | 2.4030 |
| | SBS29 | Tobacco | 0.0068 | 0.0882 | 1.5384 | 1.0089 | 2.3458 |
| LUSC | SBS1 | Endogenous clock-like age | 0.2526 | 0.5205 | 3.9657 | 1.0116 | 15.5467 |
| | SBS23 | Unknown | 0.3356 | 0.5686 | 2.2129 | 1.3928 | 3.5159 |
| | SBS25 | Chemotherapy | 0.0411 | 0.0949 | 1.6952 | 1.0646 | 2.6992 |
| OV | _all_{—} | Stage missing | | | | | |
| PAAD | SBS32 | AZA Chemotherapy | 0.1111 | 0.8842 | 4.2668 | 1.2007 | 15.163 |
| SARC | _all_{—} | Stage missing | | | | | |
| SKCM | SBS4 | Tobacco | 0.0039 | 0.9926 | 1.6503 | 1.271 | 2.1429 |
| | SBS7c | Ultraviolet | 0.3486 | 0.8503 | 0.7935 | 0.6377 | 0.9874 |
| | SBS10b | Polymerase epsilon | 0.7141 | 0.6232 | 0.8091 | 0.656 | 0.998 |
| | | mutation | | | | | |
| | SBS36 | Base excision repair MUTY | 0.3444 | 0.9259 | 1.5259 | 1.0355 | 2.2485 |
| | SBS38 | Ultraviolet | 0.0334 | 0.0264 | 0.5749 | 0.3437 | 0.9618 |
| STAD | SBS1 | Endogenous clock-like age | 0.0106 | 0.7954 | 0.8651 | 0.6997 | 1.0697 |
| | SBS7c | Ultraviolet | 0.0154 | 0.7877 | 1.2383 | 0.9872 | 1.5531 |
| | SBS16 | Unknown | 0.0308 | 0.6479 | 0.6958 | 0.4827 | 1.0000 |
| | SBS19 | Unknown | 0.0072 | 0.1246 | 1.0700 | 0.8730 | 1.3115 |
| | SBS20 | Mismatchrepair + POLD1mut | 0.0845 | 0.0143 | 0.8688 | 0.6594 | 1.1447 |
| | SBS26 | Mismatchrepair | 0.0303 | 0.6375 | 0.8767 | 0.6264 | 1.2190 |
| | SBS29 | Tobacco | 0.0500 | 0.1886 | 0.9649 | 0.7883 | 1.1810 |
| | SBS34 | Unknown | 0.0089 | 0.7431 | 1.1945 | 0.9546 | 1.4946 |
| | SBS39 | Unknown | 0.0280 | 0.4674 | 1.1622 | 0.9439 | 1.4309 |
| UCEC | _all_{—} | Stage missing | | | | | |

Included are those signatures deemed significant at p-value < false discovery rate threshold
All relationships adjusted for age and sex.
*BLCA omitted due to only n = 2 cancers diagnosed at Stage I
**p-values for trend in unit of mutational signature in relation to stage 3 and 4 combined, vs. stage 1 and 2 combined
***Relative risk calculated utilizing the cutpoint determined using maximally selected rank statistics

### Contribution of Mutational Signatures and TMB to DSS

The concordance index (c-index) calculated for a Cox proportional hazards model for DSS that included age at diagnosis, sex and tumor stage (baseline model), was compared to that calculated for models that also included mutational signatures, and recalculated again with addition of tumor mutational burden (TMB). For included tumors, mutational signatures added significantly to the c-index, indicating that the signatures contributed to prediction of DSS, except for 3 cancers in which no signatures met the false discovery rate (FDR) requirement (Table 4). For 10 cancers, TMB had also contributed to survival discrimination in the baseline model analysis (exceptions were CESC, HNSC, LGG, LUSC, PAAD, and SARC). When TMB was then added to models that incorporated clinical factors and signatures, TMB did not add further prognostic discrimination, as measured by the c-index. For many cancers, the final c-index exceeded that attained in published clinical models that included many factors not available in TCGA, suggesting that inclusiont of those clinical factors alone will not account for the higher discrimination of mutational signatures in DSS.

TABLE 4
Survival models including clinical factors only, and effect of addition of mutational signatures
and tumor mutational burden (TMB), as measured by the concordance Index (c-index).

| | | | | | Likelihood | | | |
|---|---|---|---|---|---|---|---|---|
| | | | | Model | Ratio p- | | | |
| | | | | Concordance | value - | Model | Likelihood | TMB |
| | | Concordance | | Clinical | Addition | Concordance | Ratio p- | association |
| | | Clinical | Signatures (p < | Factors | of | Clinical + | value - | with DSS |
| | Clinical | Factors | False Discovery | (col B) + | Signatures | Signatures | Addition | (adjusted |
| | Factors | (col B) | Rate (FDR) | Signatures | (Model | (col E) + | of TMB | for clinical |
| Cancers | available: | only | Threshold) | (col D) | col E) | TMB | (col G) | only) |
| BLCA | Stage\hat{ }, Age, | 0.64 | SBS1, 2, 7a, 10b, | 0.76 | <.0001 | 0.76 | 0.92 | p < .05 |
| | Sex, Grade | | 13, 22, 23, 39 | | | | | |
| | Stage, Age, | | | | | | | |
| BRCA | Sex | 0.76 | SBS10b, 12, 20, 22, 30 | 0.8 | 0.0001 | 0.8 | 0.06 | p < .05 |
| CESC | Age, Grade | 0.57 | SBS7a, 34, 42 | 0.67 | <.0001 | 0.67 | 0.63 | No |
| | Stage, Age, | | | | | | | |
| COAD | Sex | 0.75 | SBS9, 17b, 25, 26, 36 | 0.84 | <.0001 | 0.84 | 0.6 | p < .05 |
| | Stage, Age, | | | | | | | |
| HNSC | Sex, Grade | 0.58 | SBS9, 85 | 0.59 | 0.004 | 0.6 | 0.08 | p < .05 |
| | Age, Sex, Grade | | | | | | | |
| LGG | Stage, Age, | 0.79 | SBS1, 8, 12, 25 | 0.83 | <.0001 | 0.83 | 0.14 | No |
| LIHC | Sex, Grade | 0.72 | SBS1, 4, 6, 7a, 7c, 15, | 0.8 | <.0001 | 0.8 | 0.12 | p < .05 |
| | | | 19, 21, 24, 39, 85 | | | | | |
| LUAD | | | No p < FDR | | | | | p < .05 |
| LUSC | | | No p < FDR | | | | | No |
| OV | Age, Grade | 0.61 | SBS7a, 39 | 0.63 | <.0001 | 0.63 | 0.22 | p < .05 |
| | Stage, Age, | | | | | | | |
| PAAD | Sex, Grade | 0.60 | SBS8, 44 | 0.65 | <.0001 | 0.65 | 0.37 | No |
| SARC | Age, Sex | | No p < FDR | | | | | No |
| | Stage, Age, | | SBS1, 4, 7a, 7b, 7c, | | | | | |
| | | | 7d, 10b, 13, 17a, 25, | | | | | |
| SKCM | Sex | 0.56 | 31, 34, 39 | 0.67 | <.0001 | 0.67 | 0.887 | p < .05 |
| | Stage, Age, | | | | | | | |
| STAD | Sex, Grade | 0.64 | SBS10b | 0.67 | 0.0003 | 0.67 | 0.37 | p < .05 |
| UCEC | Age, Grade | 0.71 | SBS1, 6, 10a, 10b, 13, | 0.84 | <.0001 | 0.84 | 0.29 | p < .05 |
| | | | 14, 15, 16, 17a, 17b, | | | | | |
| | | | 18, 20, 22, 26, 29, 33, | | | | | |
| | | | 34, 38, 39 | | | | | |

Signatures included if p-value<false discovery threshold

### Abbreviations

FDR—False Discovery Rate
TMB—Tumor Mutational Burden

TABLE 5
Modification of the effect of radiotherapy on
cancer survival by mutational signatures, among
those with Stage II or later disease.

| Signature | | | | | | |
|---|---|---|---|---|---|---|
| Present | | NonEvents | Events | Hazard | | p-value |
| Yes | Rad | n | n | Ratio | 95% CI | interaction |
| SBS7c: | | | | | | .045 |
| Yes | Yes | 15 | 7 | 1.20 | 0.53-2.72 | |
| No | Yes | 597 | 148 | 0.78 | 0.60-1.00 | |
| Yes | No | 128 | 40 | 0.61 | 0.41-0.90 | |
| SBS24 | | | | | | .031 |
| Yes | Yes | 183 | 66 | 1.11 | 0.78-1.58 | |
| No | Yes | 429 | 89 | 0.70 | 0.52-0.95 | |
| Yes | No | 618 | 240 | 1.07 | 0.85-1.36 | |
| SBS29 | | | | | | .0038 |
| Yes | Yes | 66 | 32 | 1.43 | 0.92-2.21 | |
| No | Yes | 546 | 123 | 0.70 | 0.53-0.93 | |
| Yes | No | 275 | 86 | 0.88 | 0.63-1.22 | |
| SBS37 | | | | | | .05 |
| Yes | Yes | 52 | 8 | 0.44 | 0.16-1.18 | |
| No | Yes | 560 | 147 | 0.87 | 0.67-1.12 | |
| Yes | No | 164 | 69 | 1.27 | 0.92-1.76 | |
| SBS42 | | | | | | .028 |
| Yes | Yes | 75 | 23 | 1.30 | 0.77-2.19 | |
| No | Yes | 537 | 132 | 0.76 | 0.58-0.99 | |
| Yes | No | 264 | 111 | 1.00 | 0.76-1.31 | |

Adjusted for age, gender, type of cancer, and immortal person-time. Also restricted to TCGA participants who lived 365 days or longer, and to Stage 2 pathologic stage or later.

The joint effects of five signatures (SBS7c, SBS24, SBS29, SBS37, SBS42) and radiotherapy differed from that expected on a multiplicative scale. In patients with four signatures (SBS7c, SBS24, SBS29, SBS42), radiotherapy did not decrease risk of mortality. In patients with signature SBS37, radiotherapy decreased risk of mortality more than expected. These results suggest that the presence of particular signatures in the genetic background of the tumor may be predictive, that is, influence response to therapy, as well as prognostic, providing information about overall survival.

### Discussion

The understanding of contributions to cancer survival by specific mutagen exposure and unrepaired damage to DNA is minimal. Mutational signatures have not previously been comprehensively evaluated in relationship to clinical outcomes (stage, survival), thus the present findings provide evidence for clinical utility for such signatures. The findings suggest that signatures that are risk factors for incidence, such as mismatch repair, will not necessarily act similarly in the survival setting, and may differ in relation to disease-specific survival in important ways. Mutational signatures of carcinogen damage, and of endogenous processes such as DNA repair, were strongly associated with cancer survival. Such damage, and lack of repair, points to underlying cellular events that may drive tumor growth and proliferation, lack of responsiveness to cell cycle/apoptotic signals, and other mechanisms associated with poor prognosis. The results also indicate that tumor mutational burden may be decomposed into constituents that more strongly predict survival than the overall TMB measure. Signatures not previously linked to survival outcomes may suggest new pathogenic mechanisms. Taken together, the findings open new prospects on the understanding of survival determinants and may eventually present opportunities to provide more precisely-tailored care.

Often, risk factors for survival differ from those for incident cancer. In addition, as the same risk factor can be associated with increased risk of one cancer but reduced risk of another, it was unlikely for that to differ in the survival context. Thus, it was not unexpected that mutational signatures associated with disease incidence would have disparate relationships to disease-specific survival, in identity, magnitude, or direction. DNA repair and UV exposure are notable examples in this study: individuals with greater UV exposure have an increased melanoma incidence in the literature¹⁹, but decreased melanoma mortality in this study as well as others (Berwick et al., 2005; Heenan et al., 1991; Rosso et al., 2008). Cancer DSS is likely to involve mechanisms directly facilitating metastasis, including intravasation of blood vessels and establishment of a metastatic niche at a distant organ site, which are clearly distinct from cancer incidence processes (Valastyan & Weinberg, 2011). Thus, as metastasis is the most common cause of disease-specific mortality, endogenous and exogenous mutational processes involved in cancer initiation are expected to act somewhat differently to influence survival.

While endogenous signatures such as those of clock-like aging and APOBEC have only rarely been investigated in association with DSS (Chen et al., 2017), the prognosis associated with endogenous DNA repair defects has been more extensively studied. Deficiencies in DNA repair, such as those in mismatch and homologous repair, inherited as mutations in genes such as MLH1, PMS2, and BRCA1/2, cluster in families and predispose to increased cancer incidence. However, their relationship with cancer survival is more complex. In a number of studies, patients with Lynch Syndrome I/II, associated with inherited mismatch repair (MMR) mutations, have had improved cancer prognosis (Gryfe et al., 2000), possibly due to enhanced response to therapy (Sinicrope et al., 2011). Lack of MMR and concomitant reduced DNA repair may lead to increased cancer cell death in chemotherapy treatment, facilitating increased survival. Mismatch repair deficiencies also favor improved survival in immune checkpoint inhibitor therapy (Le et al., 2015). In contrast, BRCA 1/2 patients, whose mutations confer homologous repair deficiencies, have had similar cancer survival as unaffected individuals in many (Goodwin et al., 2012; Lee et al., 1999; Verhoog et al., 1999; Verhoog et al., 1998; Copson et al., 2018) but not all (Castro et al., 2013; Schmidt et al., 2017) studies. The present results suggest that signatures of increased MMR (some of which may be due to inherited genetic alterations, some somatically acquired) are not uniformly associated with prognosis. In LIHC and STAD, individuals with MMR signatures have a reduced risk of disease-specific mortality, relative to unaffected individuals. In other tumors (BRCA, COAD), MMR deficiency signatures are associated with increased mortality. Lack of information on treatment, specifically chemotherapy, does not allow stratification to further explore mechanistic understanding. The present findings also suggest that COAD and LGG patients with a base excision repair deficiency signature have increased disease-specific mortality. As inheritance of contributing factors is rare; it is likely that most endogenous signatures above were somatically acquired.

Effects of signatures of exogenous mutagens in many cases mirror those from human studies of carcinogen exposure, lending validity to prognostic risk factor findings, and vice-versa. As one illustration, UV light exposure has been associated with decreased risk of bladder cancer incidence (Lin et al., 2012) and mortality (Chen et al., 2010a) as well as reduced cervical cancer mortality (Chen et al., 2010a). Individuals with greater exposure to UV light have an increased incidence of melanoma (Lin et al., 2012) but often have reduced melanoma mortality (Berwicke t al., 2005; Heenan et al., 1991; Rosso et al., 2008), although evidence from ecological studies has been less consistent (Garland et al., 2003; Lachiewicz et al., 2008; Jemal et al., 2000). The three signatures of UV related to melanoma mortality in this study (SBS7a, 7b, 7c) were each associated with diminished risk. Enhanced Vitamin D serum levels have been implicated as a mechanism in the reduced mortality (Newton-Bishop et al., 2009; Hardie et al., 2020). For LIHC, however, patients bearing either of two signatures of UV-related DNA damage in tumors (SBS7a, SBS38) had an elevated mortality risk, which has not been identified in the literature. Individuals carrying tobacco-related DNA-damage signatures (Hollstein et al., 2017) in tumors had an increased mortality risk in BRCA, LGG, and SKCM, each with some support from findings from prognostic risk factor studies (Hardie et al., 2020; Abdel-Rahman & Cheung, 2018; Passarelli et al., 2016; Hou et al., 2016; McLaughlin et al., 1995; Newton-Bishop et al., 2015). While smoking both has (Hardie et al., 2020; Newton-Bishop et al., 2015) and has not (DeLancey et al., 2011; Givson et al., 2020) been related to increased melanoma mortality, in this study individuals with smoking-associated signatures were diagnosed with later melanoma stage, and a discrete measure of smoking-related damage exhibited a significant trend with DSS. The tobacco-related associations noted in this study omit a few previously reported in the literature, including those excluded due to failure to exceed the FDR threshold. Aristolochic acid, an herbal medicine additive (Poon et al., 2013), has been implicated in urinary tract but not specifically bladder cancer prognosis (Wang et al., 2019). An initial relationship with bladder cancer, however, may have come to light due to mutational signatures (Poon et al., 2015), illustrating the potential richness of reciprocal exchanges between mutational signature findings and risk factor investigations. Among other exogenous mutagen relationships identified, aflatoxin has been associated with liver (Chen et al., 2013) and non-liver cancer mortality (Hayes et al., 1984), while chemotherapy signatures indicate an earlier, advanced stage primary tumor, which has an established relationship with prognosis (Wang et al., 2020; Jegu et al., 2015).

While the findings of increased DSS in some tumors but reduced in others for the same carcinogen or signature may initially seem at odds, it is important to note that UV and smoking, for example, have similar relationships with disease incidence: UV exposure appears to reduce risk of many tumors (Lin et al., 2012; Boscoe & Schymura, 2006), but not melanoma (Lin et al., 2012), and smoking has been associated with reduced incidence of both endometrial cancer (Lindemann et al., 2008; Brinton et al., 1993) and Parkinson's disease (Checkoway et al., 2002; Chen et al., 2010b). Thus, opposing directions of signature-mortality relationships by cancer is not unexpected, but requires further validation. In all instances cited above, the associations identified in the literature are weaker than those in this study, suggesting that cutpoints identified by maximally selected rank statistics will possibly be subject to “regression to the mean”-like declines with further investigation, or that self-report of smoking, UV, or other carcinogenic exposures are subject to non-differential misclassification and other measurement error, or both. Signatures detected in this study are also those not repaired by physiologic mechanisms, implying a role for DNA repair and for threshold effects. In sum, these findings suggest that biologically-based measures of carcinogen exposure in tumors, when fully validated, will augment and clarify relationships in the literature, as well as provide hypotheses for investigation.

Individuals with high tumor mutational burden (TMB) have had a mixed prognosis in previous studies, often tumor type- and treatment-dependent (Shao et al., 2020; Samstein et al., 2019; Rivierre et al., 2020). Higher TMB is associated with improved overall survival in those who received immune checkpoint inhibitors (ICI) (Altan et al., 2017) therapy, which received FDA approval after recruitment for TCGA. Defects in DNA mismatch repair in particular have been correlated with TMB (Chalmers et al., 2017). In at least one analysis, TMB has been only weakly associated with survival after inclusion of other contributing factors, including mutations in DNA repair genes (Aoude et al., 2020). SBS signatures may serve, in part, as a surrogate but potentially stronger measure of TMB because they are a summary measure of pathogenic mutations, gene methylation, and other potentially contributing events (Chen et al., 2017; Chalmers et al., 2017; Aoude et al., 2020; Chen et al., 2020c). The present analysis suggests that the relationship between individual signatures and DSS varies uniquely for each tumor, however, thus it is unlikely that TMB is the underlying foundation of all relationships between signatures and DSS. The present multivariable analysis results also indicate that SBS signatures are contributing to cancer survival independently of TMB, while TMB is rarely contributing to DSS independently of signatures, setting the stage for further investigation of their distinct role.

Signatures of many carcinogens have not been identified, and conversely, the etiology of many recognized mutational signatures is as yet unclear. Thus, 24 of 80 signatures (30%) related strongly to DSS are of unknown origin. In addition, despite restricting cancers to those with >50 events and cutpoints to cell sizes>5, some signatures are based on small cells, and may be false positive findings. Analyses of continuous, discrete, and stage-specific measures were conducted in part to provide a broader basis for evaluation of these findings. The FDR was also utilized, and reported findings limited to only those that exceeded the FDR threshold. The large sample sizes overall, follow-up for several years, and availability of numerous signatures with potential prognostic utility are among the study strengths.

Mutational signatures contain substantial promise for enhancing mechanistic understanding, outcome discrimination, and for confirmation of prognostic risk factor findings, as well as for generation of novel hypotheses. SBS signatures require validation in tumor sequencing studies before undergoing further consideration for clinical use.

### Abbreviations

ACC Adrenocortical carcinoma
BLCA Bladder Urothelial Carcinoma
LGG Brain Lower Grade Glioma
BRCA Breast invasive carcinoma
CESC Cervical squamous cell carcinoma and
endocervical adenocarcinoma
CHOL Cholangiocarcinoma
COAD Colon adenocarcinoma
ESCA Esophageal carcinoma
GBM Glioblastoma multiforme
HNSC Head and Neck squamous cell carcinoma
KICH Kidney Chromophobe
KIRC Kidney renal clear cell carcinoma
KIRP Kidney renal papillary cell carcinoma
LIHC Liver hepatocellular carcinoma
LUAD Lung adenocarcinoma
LUSC Lung squamous cell carcinoma
DLBC Lymphoid Neoplasm Diffuse Large B-cell b
Lymphoma
MESO Mesothelioma
OV Ovarian serous cystadenocarcinoma
PAAD Pancreatic adenocarcinoma
PCPG Pheochromocytoma and Paraganglioma
PRAD Prostate adenocarcinoma
READ Rectum adenocarcinoma
SARC Sarcoma
SKCM Skin Cutaneous Melanoma
STAD Stomach adenocarcinoma
TGCT Testicular Germ Cell Tumors
THYM Thymoma
THCA Thyroid carcinoma
UCS Uterine Carcinosarcoma
UCEC Uterine Corpus Endometrial Carcinoma

### Signature Definitions

SBS1=Endogenous clock-like age
SBS2=Endogenous Apobec3a
SBS3=Homologous repair
SBS4=Tobacco
SBS5=Endogenous clock-like age
SBS6=Mismatchrepair
SBS7a=UV
SBS7b=UV
SBS7c=UV
SBS7d=UV
SBS8=Unknown
SBS9=PolETA Somatichypermutation
SBS10a=Polymerase epsilon hypermutations
SBS10b=Polymerase epsilon mutations
SBS11=Temozolo Chemotherapy
SBS12=Liver cancer
SBS13=Endogenous apobec
SBS14=Mismatchrepair+PolEpsil
SBS15=Mismatchrepair
SBS16=Unknown
SBS17a=Unknown
SBS17b=Unknown
SBS18=ReactiveOxy
SBS19=Unknown
SBS20=Mismatchrepair+POLD1mut
SBS21=Mismatchrepair
SBS22=Aristolochic acid
SBS23=Unknown
SBS24=Aflatoxin
SBS25=Chemotherapy
SBS26=Mismatchrepair
SBS28=Unknown
SBS29=Tobacco chewing in Hollstein
SBS30=BER repair-NTHL1 mut
SBS31=PLChemotherapy
SBS32=AZA Chemotherapy
SBS33=Unknown
SBS34=Unknown
SBS35=PLChemotherapy
SBS36=BERrepair MUTY
SBS37=Unknown
SBS38=UV
SBS39=Unknown
SBS40=Unknown
SBS41=Unknown
SBS42=Haloalkane
SBS44=Mismatchrepair
SBS84=Actind cytidinedeaminase
SBS85=Actind cytidinedeaminase
SBS86=Chemotherapy

### Example 2

It was determined whether single base substitution signatures, covering a range of environmental agent and endogenous exposures, as well as defective DNA repair pathways, were associated with cancer survival. Algorithms for mutational signature assessment were used to elucidate cancer-specific outcomes.

### Methods

Tumor exome sequencing data were derived from The Cancer Genome Atlas (TCGA). Description of cancer patient recruitment, follow-up, and ascertainment of disease outcomes has been published. TCGA somatic mutation data of 10,179 patients (reference genome GRCh38) from 33 cancer types were downloaded from the Genomic Data Commons. Eligible cancers were those with at least n=50 disease-specific survival (DSS) events, and only TCGA participants with DSS outcomes that were deemed appropriately defined¹⁰ were retained. Stage 0 cancers were also omitted, and those missing stage were excluded from stage-specific analyses. The probability matrix for 49 established COSMIC reference mutational signatures (v3) was downloaded from Synapse Documentation (https://www.synapse.org/#ISynapse:syn11738319

### Signature Identification

The association of mutational signatures with endogenous processes or exogenous mutagens has been described. A catalog of 96 three-nucleotide motifs that surround the mutational focus (one upstream nucleotide+mutation site+one downstream nucleotide site) was prepared, and frequency tables of this motif catalog derived for each involved patient. A computational function from R package MutationalPatterns was used to fit the patient mutational motif frequency tables to the reference mutational signatures while requiring the coefficients, i.e., signature-to-patient contribution strengths, be non-negative values. The estimated coefficients came out in the form of a 96-by-10,179 matrix of non-negative values.

### Statistical Analysis

Disease-specific survival (DSS) was the outcome of interest. Thus, disease-specific mortality was counted as an event, and deaths from other causes (competing risks) and loss-to-follow up were censored. Time to event (cancer, other cause of death, or end of follow-up period) was calculated by TCGA as days from cancer diagnosis. To correct for disease-free immortal person-time, which is present in the time from diagnosis to recruitment, disease-specific survival time was re-calculated. As the exact surgery dates of TCGA patients are unknown, a three-month interval was assumed for patients to sustain from initial diagnosis to the recruitment date and such a three-month-period was subtracted from each patient's disease-free survival. Mutational signatures were modeled in relationship to survival as continuous or discrete (excluding zero) measures, and also using a single cutpoint determined by maximally selected rank statistics, employing a restriction that the cutpoint include cell sizes of 5 or greater. The three clinical factors commonly available for all organ sites in TCGA data, age at diagnosis, sex, and stage, were included in all analyses. Cox proportional hazards models for DSS, estimating hazard ratios (HR) and 95% confidence intervals (CI), were fit. The proportional hazards assumption was verified by Schoenfeld residuals. Correction for immortal person-time was included, and all events that occurred prior to study consent excluded. To further establish the nature of the relationship between mutational signatures and DSS, it was also determined whether stage at diagnosis differed according to signature, comparing Stage I to Stage III/IV disease, using the measures and adjustment factors employed in Cox regression, but quantifying relative risks (RR) and 95% CI.

Models that included only diagnosis age, sex, and disease stage (baseline model) were compared to a model that also included all mutational signatures significantly associated with survival, and also to a third model that incorporated in addition a measure of tumor mutational burden (TMB). Then a concordance statistic (c-statistic, commonly known as c-index) was calculated to compare improvement in model fit with sequential incorporation of these measures. The difference in c-index between the baseline and other models was assessed. Tumor mutational burden was quantified to evaluate nonsynonomous somatic mutations (references here), and included as a continuous variable. A two-tailed p-value of <0.05, after adjustment for the false discovery rate (FDR) of 0.10, was considered significant.

### Results

Of solid tumors, 14 organ sites, constituting 15 distinct pathological entities or cancer types, and 6292 patients were included in the analysis, after exclusion of those with less than 50 DSS events (Supplementary Table 1). Included cancer types ranged from n=163 for pancreatic adenocarcinoma (PAAD) to n=917 for breast carcinoma (BRCA)) (Table 1; see above). Mean age of included patients ranged from 43.2 years [Low Grade Glioma (LGG)] to 68.0 [Bladder Carcinosarcoma (BLCA)]. Signatures of 49 distinct mutational patterns of single base substitution (SBS) were examined in relationship to DSS¹².

Signatures Associated with Stage III/IV Disease

Among the nine cancers with stage information, all had signatures associated with later stage (III/IV) vs earlier (I/II), either as continuous or discrete measures, or using a single cutpoint (Table 2; see above). Risk of later stage disease was generally lower among those with the mismatch repair and ultraviolet exposure signatures. Later stage disease was more common among those with signatures of tobacco exposure and previous chemotherapy.

Signatures Associated with DSS

The number of signatures related to altered survival per tumor, after consideration of FDR ranged from none {(Lung adenocarcinoma (LUAD) Lung squamous cell (LUSC), and Sarcoma (SARC)} to 13 [Skin Cutaneous Melanoma (SKCM, Table 2)]. While results derived using continuous or discrete measures often supported those obtained using other means, only those relationships identified using a single cutpoint and evaluated using hazard ratios are described below.

Among endogenous mutation measures (FIGS. 1 and 2), the most common etiology of the mutational signatures identified was a clock-like signature associated with aging (SBS1), implicated in survival of 6 cancers, including Colon Adenocarcinoma (COAD), LGG, Liver hepatocellular carcinoma (LIHC), Ovarian Serous Cystadenocarcinoma (OV), Stomach Adenocarcinoma (STAD), and Uterine Corpus Endometrial Carcinoma (UCEC) (FIG. 1). The effect of the aging signature on survival was mixed, in that risk of cancer-specific mortality was reduced among participants with BLCA, and UCEC cancers (HR=0.50, HR=0.30, respectively), but elevated up to 3.65-fold in other cancers. Two signatures (SBS2 and SBS13) of deregulated APOBEC-related cytosine deamination activity were both associated with reduced mortality in BLCA patients (HR=0.44, HR=0.46, respectively), but in BRCA, risk of disease-specific mortality was elevated, using continuous or discrete measures. Defects in DNA repair mechanisms that may have contributed to altered survival include several signatures of mismatch repair (SBS14, 20, 26) in BRCA, COAD, and UCEC tumors, in which BRCA and COAD patients had up to 3.2-fold increased mortality risk, but mortality in UCEC was substantially reduced (HR=0.13). A base excision repair signature (SBS30) was related to 1.5, 1.74, and 2.6 fold increased mortality, respectively, in BLCA, LGG, and BRCA. Polymerase epsilon, which acts in both replication and DNA recombination, also facilitates completion of base excision and nucleotide excision repair, thus signatures of defective polymerase epsilon (SBS10a, SBS10b) in BLCA, BRCA, SKCM, or STAD survival could be attributed to these several cellular mechanisms. In addition to the above, a further endogenous process implicated in DSS is activation-induced cytidine deaminase in 3.5 and 5-fold increased risks of disease-specific mortality in LIHC and Head and Neck Squamous Cell Carcinoma (HNSC).

Among signatures of exogenous mutagenic agents (FIGS. 3 and 4), that associated with aristolochic acid, an herbal medicine component (SBS22), was related to reduced mortality in BLCA (HR=0.33) (Table 2). Ultraviolet (UV) exposure, as captured by the SBS7a, 7b, 7c signatures, was related to reduced mortality in BLCA, OV and SKCM cancers (HR=0.35-0.56; Table 2). Those with signatures of tobacco exposure¹⁷ (SBS4, SBS29) had increased risks of cancer mortality in BRCA (4-fold), LGG (2.2 to 2.8-fold), and SKCM (3-fold). COAD, LGG, LUSC, and SKCM patients bearing a signature of previous chemotherapy treatment (SBS11, SBS25, SBS31) had 4 to 5-fold elevated risks of mortality due to their respective cancers, while chemotherapy-related mortality was elevated 2.5-fold in BLCA. CESC and COAD-specific mortality were increased 3-8-fold among those whose cancers demonstrated the mutational signatures of haloalkanes (SBS42), an organic solvent exposure incurred occupationally¹⁸. Aflatoxin mutagenesis (SBS24) was associated with a 3-fold increased risk of LGG-specific mortality and a greater than 13-fold increased risk of mortality among those with LIHC. Among COAD patients, those with a signature of reactive oxygen species (SBS18), considered a metabolite of carcinogenic exposures but also generated endogenously, had a 3.5-fold increased risk of disease-specific mortality.

To evaluate one possible mechanism whereby mutational signatures might influence survival, we assessed the relationship between signature and stage within each cancer. Of the n=46 evaluable relationships, 10 were also associated with stage at diagnosis when assessed using continuous, discrete, or the binary survival cutpoint measures. However, many with strong relationships with DSS (continuous or discrete, and binary cutoff) were not associated with stage at diagnosis (COAD and SBS26 (mismatch repair), LIHC and SBS1 (Endogenous clock-like age), SKCM and SBS7b (UV), and SKCM and SBS10b (Polymerase Epsilon) are among those).

### Contribution of Mutational Signatures and TMB to DSS

The concordance index (c-index) calculated for a Cox proportional hazards model for DSS that included age at diagnosis, sex and tumor stage (baseline model) was compared to that calculated for models that also included mutational signatures, and recalculated again with the addition of tumor mutational burden (TMB). For all 15 included tumors, mutational signatures added additional prognostic discrimination to the c-index, indicating that the signatures contributed to prediction of DSS (Table 4). For 9 cancers, TMB had also contributed to survival discrimination in the baseline model analysis (exceptions were CESC, HNSC, LGG, LUSC, PAAD, and SARC). When added to models that incorporated clinical factors and signatures, TMB did not add further prognostic discrimination, as measured by the c-index (Table 4) For many cancers, the final c-index exceeded that attained in clinical models that included many factors not available in TCGA, suggesting that inclusion of those clinical factors alone will not account for the higher discrimination of DSS.

### Discussion

An understanding of contributions to cancer survival by specific mutagen exposure and unrepaired damage to DNA is minimal. Mutational signatures have not previously been comprehensively evaluated in relationship to clinical outcomes (stage, survival), thus our findings provide evidence for clinical utility for such signatures. The findings suggest that signatures that are risk factors for incidence, such as mismatch repair, will not necessarily act similarly in the survival setting, and may differ in relation to disease-specific survival in important ways. It was found that mutational signatures of carcinogen damage, and of endogenous processes such as DNA repair, were strongly associated with cancer survival. Such damage, and lack of repair, points to underlying cellular events that may drive tumor growth and proliferation, lack of responsiveness to cell cycle/apoptotic signals, and other mechanisms associated with poor prognosis. The results also indicate that tumor mutational burden may be decomposed into constituents that more strongly predict survival than the overall TMB measure. Signatures not previously linked to survival outcomes may suggest new pathogenic mechanisms. Taken together, the findings open new prospects on our understanding of survival determinants and may eventually present opportunities to provide more precisely-tailored care.

Often, risk factors for survival differ from those for incident cancer. In addition, as the same risk factor can be associated with increased risk of one cancer but reduced risk of another, it was unlikely for that to differ in the survival context. Thus, it was not unexpected that mutational signatures associated with disease incidence would have disparate relationships to disease-specific survival, in identity, magnitude, or direction. DNA repair and UV exposure are notable examples in this study: individuals with greater UV exposure have an increased melanoma incidence in the literature, but decreased melanoma mortality in this study as well as others. Cancer DSS is likely to involve mechanisms directly facilitating metastasis, including intravasation of blood vessels and establishment of a metastatic niche at a distant organ site, which are clearly distinct from cancer incidence processes. Thus, as metastasis is the most common cause of disease-specific mortality, endogenous and exogenous mutational processes involved in cancer initiation are expected to act somewhat differently to influence survival.

While endogenous signatures such as those of clock-like aging and APOBEC have only rarely been investigated in association with DSS, the prognosis associated with endogenous DNA repair defects has been more extensively studied. Deficiencies in DNA repair, such as those in mismatch and homologous repair, inherited as mutations in genes such as MLH1, PMS2, and BRCA1/2, cluster in families and predispose to increased cancer incidence. However, their relationship with cancer survival is more complex. In a number of studies, patients with Lynch Syndrome I/II, associated with inherited mismatch repair (MMR) mutations, have had improved cancer prognosis, possibly due to enhanced response to therapy. Lack of MMR and concomitant reduced DNA repair may lead to increased cancer cell death in chemotherapy treatment, facilitating increased survival. Mismatch repair deficiencies also favor improved survival in immune checkpoint inhibitor therapy. In contrast, BRCA 1/2 patients, whose mutations confer homologous repair deficiencies, have had similar cancer survival as unaffected individuals in many but not all studies. The present results suggest that signatures of increased MMR (some of which may be due to inherited genetic alterations, some somatically acquired) are not uniformly associated with prognosis. In LIHC and STAD, individuals with MMR signatures have a reduced risk of disease-specific mortality, relative to unaffected individuals. In other tumors (BRCA, COAD), MMR deficiency signatures are associated with increased mortality. Lack of information on treatment, specifically chemotherapy, does not allow stratification to further explore mechanistic understanding. Our findings also suggest that COAD and LGG patients with a base excision repair deficiency signature have increased disease-specific mortality. As inheritance of contributing factors is rare; it is likely that most endogenous signatures above were somatically acquired.

Effects of signatures of exogenous mutagens in many cases mirror those from human studies of carcinogen exposure, lending validity to prognostic risk factor findings, and vice-versa. As one illustration, UV light exposure has been associated with decreased risk of bladder cancer incidence and mortality as well as reduced cervical cancer mortality. Individuals with greater exposure to UV light have an increased incidence of melanoma but often have reduced melanoma mortality, although evidence from ecological studies has been less consistent. The three signatures of UV related to melanoma mortality in this study (SBS7a, 7b, 7c) were each associated with diminished risk. Enhanced Vitamin D serum levels have been implicated as a mechanism in the reduced mortality. For LIHC, however, patients bearing either of two signatures of UV-related DNA damage in tumors (SBS7a, SBS38) had an elevated mortality risk, which has not been identified in the literature. Individuals carrying tobacco-related DNA-damage signatures in tumors had an increased mortality risk in BRCA, LGG, and SKCM, each with some support from findings from prognostic risk factor studies. While smoking both has and has not been related to increased melanoma mortality, in this study individuals with smoking-associated signatures were diagnosed with later melanoma stage, and a discrete measure of smoking-related damage exhibited a significant trend with DSS. The tobacco-related associations noted in this study omit a few previously reported in the literature, including those excluded due to failure to exceed the FDR threshold. Aristolochic acid, an herbal medicine additive, has been implicated in urinary tract but not specifically bladder cancer prognosis. An initial relationship with bladder cancer, however, may have come to light due to mutational signatures, illustrating the potential richness of reciprocal exchanges between mutational signature findings and risk factor investigations. Among other exogenous mutagen relationships identified, aflatoxin has been associated with liver and non-liver cancer mortality, while chemotherapy signatures indicate an earlier, advanced stage primary tumor, which has an established relationship with prognosis.

While the findings of increased DSS in some tumors but reduced in others for the same carcinogen or signature may initially seem at odds, it is important to note that UV and smoking, for example, have similar relationships with disease incidence: UV exposure appears to reduce risk of many tumors but not melanoma, and smoking has been associated with reduced incidence of both endometrial cancer and Parkinson's disease. Thus, opposing directions of signature-mortality relationships by cancer is not unexpected, but requires further validation. In all instances cited above, the associations identified in the literature are weaker than those in this study, suggesting that cutpoints identified by maximally selected rank statistics will possibly be subject to “regression to the mean”-like declines with further investigation, or that self-report of smoking, UV, or other carcinogenic exposures are subject to non-differential misclassification and other measurement error, or both. Signatures detected in this study are also those not repaired by physiologic mechanisms, implying a role for DNA repair and for threshold effects. In sum, these findings suggest that biologically based measures of carcinogen exposure in tumors, when fully validated, will augment and clarify relationships in the literature, as well as provide novel hypotheses for further investigation.

Individuals with high tumor mutational burden (TMB) have had a mixed prognosis in previous studies, often tumor type- and treatment-dependent. Higher TMB is associated with improved overall survival in those who received immune checkpoint inhibitor (ICI) therapy, which received FDA approval after recruitment for TCGA. Defects in DNA mismatch repair in particular have been correlated with TMB. In at least one analysis, TMB was only weakly associated with survival after inclusion of other contributing factors, including mutations in DNA repair genes. SBS signatures may serve, in part, as a surrogate but potentially stronger measure of TMB because they are a summary measure of pathogenic mutations, gene methylation, and other potentially contributing events. The present analysis suggests that the relationship between individual signatures and DSS varies uniquely for each tumor; however, it is thus unlikely that TMB is the underlying foundation of all relationships between signatures and DSS. The multivariable analysis results also indicate that SBS signatures are contributing to cancer survival independently of TMB, while TMB is rarely contributing to DSS independently of signatures, setting the stage for further investigation of their distinct roles.

Signatures of many carcinogens have not been identified, and conversely, the etiology of many recognized mutational signatures is as yet unclear. Thus, 24 of 80 signatures (30%) related strongly to DSS are of unknown origin. In addition, despite restricting cancers to those with n>50 events and cutpoints to cell sizes≥5, some signatures are based on small cells, and may be false positive findings. We conducted analyses of continuous, discrete, and stage-specific measures in part to provide a broader basis for evaluation of these findings. The FDR was utilized, and limited reported findings to only those that exceeded the FDR threshold. The large sample sizes overall, follow-up for several years, and availability of numerous signatures with potential prognostic utility are among the study strengths. Mutational signatures contain considerable promise for enhancing mechanistic understanding, for outcome discrimination, and for confirmation of prognostic risk factor findings, as well as for generation of novel hypotheses.

### Example 3

### Exemplary Uses of Signatures in Clinical Medicine

Example A. Keytruda (generic name Pembrolizumab) is a monoclonal antibody that blocks the PD-1/PD-L1 pathway, and is FDA-approved for treatment in children or adults with unresectable or metastatic solid tumors (with the exception of central nervous system), that have progressed following treatment, and that have no satisfactory therapy options. For patients in that context, an additional requirement for therapy eligibility is tumor mutational burden (TMB), as determined by the FDA-approved test F1CDx. In the study described in Example 1, the mutational signatures for all 16 solid tumors exceed TMB in prediction of disease-specific survival (DSS) using the concordance index, regardless of whether TMB is measured on a continuous scale or modeled as a dichotomous variable. Thus, specific signatures for each cancer, or several in combination, may be more useful to determine treatment eligibility for Keytruda than the existing FDA-approved TMB test.

Tumor mutational burden is currently tested using the F1CDx™ assay from Foundation Medicine or via next-generation sequencing panels from select commercial reference laboratories. FDA-approved assessment for TMB may be readily adaptable to assessment for the mutational signatures disclosed herein. Patients with higher TMB have had a more favorable response to PD1/PD-L1 blockade in numerous cancers in the literature (Goodman 2017, McGrail 2021) thus the mutational signatures identified herein may allow additional FDA-approved indications for Keytruda. The mutational signatures in this invention may be particularly relevant to Keytruda use in Melanoma, one of the cancers include in this invention. Melanoma is an example where the deconvolution of TMB into component mutational signatures may refine and hone therapy benefit, as the signatures related to reduced mortality risk might or might not be useful to define individuals who may not benefit from Immune checkpoint therapy. Thus, use of mutational signatures may be particularly useful in determining benefit from Keytruda in brain tumors, bladder cancers and head and neck cancers.

Example B. In several studies, TMB is a predictor of response to not only Keytruda, but also Opdivo (Nivolumab) in non-small cell lung cancer (NSCLC). In the study in Example 1, mutational signatures did not pass a threshold for false discovery rate for subsets of lung cancer (LUAD and LUSC) that make up NSCLC. However, statistical power was restricted due to the use of subsets by TCGA, and mutational signatures disclosed herein may be useful to determine benefit from these therapies in individuals with NSCLC.

Example C. The immune checkpoint inhibitors cemiplimab, atezolimab, avelumab, and durvalumab are not currently approved for use in conjunction with a TMB assay but by their mechanism, may very well prove to be most beneficial in those with high TMB. Thus, the use of the signatures disclosed herein may well help determine clinical benefit with these treatments as well.

### Example 4

Individuals carrying tobacco-related DNA-damage signatures (Hollstein et al., 2017) in tumors had an increased mortality risk in breast (BRCA), low grade glioma (LGG), and melanoma (SKCM), each with some support from findings from prognostic risk factor studies (Hardie et al., 2020; Abdel-Rahman & Cheung, 2018; Passarelli et al., 2018; Hou et al., 2016; McLaughlin et al., 1995; Newton-Bishop et al., 2015). While smoking both has (Hardie et al., 2020; Newton-Bishop et al., 2015) and has not (DeLancey et al., 2011; Gibson et al., 2020) been related to increased melanoma mortality, in this study individuals with smoking-associated signatures were diagnosed with later melanoma stage. Tobacco-related signatures were not associated with risk of disease-specific survival in the two lung cancer subgroups included (Lung adenocarcinoma and lung squamous cell carcinoma).

Individuals with greater exposure to UV light have an increased incidence of melanoma (Lin et al., 2012) but often have reduced melanoma mortality (Berwick et al., et al., 2005; Heenan et al., 1991), although evidence from ecological studies has been less consistent (Garland et al., 2003; Lachiewicz et al., 2008; Jemal et al., 2000). We found reduced melanoma mortality in association with several UV signatures but not all in our study.

Mismatch repair (MMR) deficiency is generally associated with increased risk of cancer incidence but reduced risk of mortality, in part due to improved response to chemotherapy. Our results suggest that signatures of increased MMR (some of which may be due to inherited genetic alterations, some somatically acquired) are not uniformly associated with improved prognosis. In LIHC and STAD, individuals with MMR signatures have a reduced risk of disease-specific mortality, relative to unaffected individuals. In other tumors (breast (BRCA), colon (COAD)), MMR deficiency signatures are associated with increased mortality.

### REFERENCES

Abdel-Rahman & Cheung, Medical Oncol., 35:68 (2018).
Alexandrov et al., Nature, 500:415 (2013).
Alexandrov et al., Nature, 578:94 (2020).
Alexandrov et sl., Cur. Op. Gen. & Dev., 24:52 (2014).
Altan et al., J. Clin. Oncol.: Off. J. Am. Soc. Clin. Onc., 23:5202 (2017).
Aoude et al., Scientific Rep., 10:17687 (2020).
Benjamini et al., Behav. Brain Res., 125:279 (2001).
Berwick et al., J. Nat. Cancer Inst., 97:195 (2005).
Blokzijl et al., Genome Med., 10:33 (2018).
Boscoe & Schymura, BMC Cancer, 6:264 (2006).
Brinton et al., Am. J. Epid., 137:281 (1993).
Castro et al., J. Clin. Oncol.: Off. J. Am. Soc. Clin. Onc., 31:1748 (2013).
Catalogue of Somatic Mutations in Cancer. http://cancer.sanger.ac.uk/cosmic.
Chalmers et al., Genome Med., 9:34 (2017).
Checkoway et al., Am. J. Epid., 155:732 (2002).
Chen et al., Cancer Causes & Control: CCC, 21:1701 (2010a).
Chen et al., Cancer Prev. Res., 6:1038 (2013).
Chen et al., Nature Comm., 8:465 (2017).
Chen et al., Neurology, 74:878 (2010b).
Chen et al., Oncoimmunoloqy, 9:1788252 (2020).
Copson et al., Lancet Oncol., 19:169 (2018).
DeLancey et al., Cancer Causes & Control: CCC, 22:937 (2011).
Garland et al., Annals Epid., 13:395 (2003).
Gerstung et al., Nature, 578:122 (2020).
Gibson et al., Br. J. Dermatol., 182:1136 (2020).
Goodwin et al., J. Clin. Oncol.: Off. J. Am. Soc. Clin. Onc., 30:19 (2012).
Gryfe et al., New Eng. J. Med., 342:69 (2000).
Hardie et al., J. Invest. Dermatol., 140:327 (2020).
Harrell et al., Stat. Med., 15:361 (1996).
Hayes et al., Food Chem. Toxicol., 22:39 (1984).
Heenan et al., ______, 68:2079 (1991).
Hollstein et al., Oncogene, 36:158 (2017).
Hothorn, Comp. Stat. Data Anal., 43:127 (2003).
Hou et al., Neuro-Oncoloqv, 18:105 (2016).
Jegu et al., Oral Oncol., 51:457 (2015).
Jemal et al., J. Nat. Cancer Inst., 92:811 (2000).
Koh et al., Genome Biol., 21:37 (2020).
Lachiewicz et al., Arch. Derma., 144:515 (2008).
Le et al., New Eng. J. Med., 372:2509 (2015).
Lee et al., J. Nat. Cancer Inst., 91:259 (1999).
Lin et al., Int. J. Cancer, 131:E1015 (2012).
Lindemann et al., Br. J. Cancer, 9:1582 (2008).
Liu et al., Cell, 173:400 (2018).
McLaughlin et al., Intern. J. Cancer, 60:190 (1995).
Mimaki et al., Carcinogenesis, 37:817 (2016).
Newton-Bishop et al., Intern. J. Cancer, 136:2890 (2015).
Newton-Bishop et al., J. Clin. Oncol.: Off. J. Am. Soc. Clin. Onc., 27:5439 (2009).
Passarelli et al., J. Clin. Oncol.: Off. J. Am. Soc. Clin. Onc., 34:1315 (2016).
Poon et al., Genome Med., 7:38 (2015).
Poon et al., Sci. Transl. Med., 5:197 (2013).
Ribic et al., New Eng. J. Med., 349:247 (2003).
Riviere et al., Mol. Cancer Ther., 19:2139 (2020).
Rosso et al., Eur. J. Cancer, 44:1275 (2008).
Samstein et al., Nature Genetics, 51:202 (2019).
Schmidt et al., J. Nat. Cancer Inst., 109: (2017).
Shao et al., JAMA Netw. Open, 3e2025109 (2020).
Sinicrope et al., J. Nat. Cancer Inst., 103:863 (2011).
Staaf et al., Nature Med., 25:1526 (2019).
The Cancer Genome Atlas. http://cancergenome.nih.gov/.
Valastyan & Weinberg, Cell, 147:275 (2011).
Verhoog et al., J. Clin. Oncol.: Off. J. Am. Soc. Clin. Onc., 17:3396 (1999).
Verhoog et al., Lancet, 351:316 (1998).
Wang et al., BMC Urol., 19:24 (2019).
Wang et al., Medicine, 99:e22191 (2020).
Weinstein et al., Nat. Gen., 45:1113 (2013).
U.S. Pat. No. 10,776,718
Alexandrov et al., Nature, https://doi.org/10.1038/s41586-020-1943-3 (2020).

All publications, patents and patent applications are incorporated herein by reference. While in the foregoing specification, this invention has been described in relation to certain embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method to detect mutational signatures correlated with disease-specific survival, comprising:
detecting in a tumor sample from a cancer patient the presence of one or more mutational signatures comprising one or more of the mutational signatures in Table 2, wherein the presence of the one or more signatures in Table 2 in the sample relative to a corresponding sample without the one or more signatures in Table 2 is indicative of disease-specific survival in the patient.

2. A method to determine disease-specific survival in a cancer patient, comprising:
obtaining a tumor sample from a patient with breast cancer (BRCA), bladder cancer (BLCA), colon adenocarcinoma (COAD), brain lower grade glioma (LGG), liver hepatocellular carcinoma (LIHC), ovarian serous cystadenocarcinoma (OV), stomach adenocarcinoma (STAD), uterine corpus endometrial carcinoma (UCEC), skin cutaneous melanoma (SKC), cervical squamous cell carcinoma and endocervical adenocarcinoma (CESC), head and neck squamous cell carcinoma (HNSC), lung squamous cell carcinoma (LUSC) or pancreatic adenocarcinoma (PAAD); and
determining if the tumor sample has one or more mutational signatures comprising one or more of the mutational signatures in Table 2, wherein the presence of the one or more signatures in Table 2 in the sample relative to a corresponding sample without the one or more signatures in Table 2 is indicative of disease-specific survival in the patient.

3. The method of claim 1 or 2 wherein the one or more signatures are selected from SBS1, SBS2, SBS6, SBS10a, SBS10b, SBS13, SBS15, SBS20, SBS26, or SBS30, or any combination thereof.

4. The method of claim 1, 2 or 3 wherein the cancer is COAD, LGG, LIHC, OV, STAD, or UCEC.

5. The method of claim 1, 2 or 3 wherein the cancer is BRCA, COAD, LIHC, or STAD.

6. The method of claim 1, 2 or 3 wherein the cancer is BLCA, BRCA, LGG, SKC or STAD.

7. The method of claim 1, 2 or 3 wherein the cancer is LIHC or HNSC.

8. The method of any one of claims 1 to 7 wherein the sample is from a stage I cancer.

9. The method of any one of claims 1 to 7 wherein the sample is from a stage II/III/IV cancer.

10. The method of any one of claims 1 to 9 wherein one of the mutational signatures in Table 2 is detected.

11. The method of any one of claims 1 to 9 wherein two or more of the mutational signatures in Table 2 are detected.

12. The method of any one of claims 1 to 9 wherein up to thirteen of the mutational signatures in Table 2 are detected.

13. The method of any one of claims 1 to 12 wherein the presence of SBS1 is detected.

14. The method of any one of claims 1 to 13 wherein the presence of SBS2 or SBS13, or both, is detected.

15. The method of any one of claims 1 to 14 wherein the presence of SBS6, 15, 20, or 26, or any combination, is detected.

16. The method of any one of claims 1 to 15 wherein the presence of SBS30 is detected.

17. The method of any one of claims 1 to 16 wherein the presence of SBS10a or SBS10b, or both, is detected.

18. The method of any one of claims 1 to 17 wherein the presence of the one or more mutational signatures is indicative of increased survival.

19. The method of any one of claims 1 to 17 wherein the presence of the one or more mutational signatures is indicative of decreased survival.

20. The method of any one of claims 1 to 19 wherein the one or more mutational signatures are detected using a nucleic acid amplification reaction.

21. The method of any one of claims 1 to 20 wherein the one or more mutational signatures are detected using a probe.

22. The method of any one of claims 1 to 21 wherein the one or more mutational signatures are detected using sequencing.

23. The method of claim 22 wherein the sequencing is specific for the one or more mutational signatures.

24. The method of any one of claims 1 to 23 wherein the presence of the one or more mutational signatures is indicative of response to therapy.

25. The method of any one of claims 1 to 23 wherein the presence of the one or more mutational signatures is indicative of a need for therapy

26. The method of claim 24 or 25 wherein the therapy is radiotherapy.

27. The method of claim 24 or 25 wherein the therapy is chemotherapy.

28. The method of claim 24 or 25 wherein the therapy is immunotherapy.

29. The method of claim 28 wherein the therapy is antibody therapy.

30. A method to detect mutational signatures correlated with disease-specific survival, disease-free interval, progression-free interval, progression-free survival or overall survival, comprising:
detecting in a tumor sample from a cancer patient the presence of one or more mutational signatures comprising one or more of the mutational signatures in Table 2, or any combination thereof, wherein the presence of the one or more signatures in Table 2 in the sample relative to a corresponding sample without the one or more signatures in Table 2, is indicative of disease-specific survival, disease-free interval, progression-free interval, progression-free survival or overall survival in the patient.

31. The method of claim 30 wherein the presence of the one or more mutational signatures is indicative of response to therapy.

32. The method of claim 30 wherein the presence of the one or more mutational signatures is indicative of a need for therapy

33. The method of claim 31 or 32 wherein the therapy is radiotherapy.

34. The method of claim 31 or 32 wherein the therapy is chemotherapy.

35. The method of claim 31 or 32 wherein the therapy is immunotherapy.

36. The method of claim 34 wherein the therapy is antibody therapy.

37. A kit comprising one or more primers or one or more probes specific for detecting the one or more of the mutational signatures in Table 2.

38. A microarray comprising one or more probes specific for detecting the one or more of the mutational signatures in Table 2.

39. A method to treat cancer in a human, comprising: administering an anti-cancer therapy to a human having a tumor comprising one or more of the mutational signatures in Table 2 that is/are indicative of decreased disease-specific survival, shorter disease-free interval, shorter progression-free interval, shorter progression-free survival or decreased overall survival.

40. The method of claim 39 wherein the human has breast cancer (BRCA), bladder cancer (BLCA), colon adenocarcinoma (COAD), brain lower grade glioma (LGG), liver hepatocellular carcinoma (LIHC), ovarian serous cystadenocarcinoma (OV), stomach adenocarcinoma (STAD), uterine corpus endometrial carcinoma (UCEC), skin cutaneous melanoma (SKC), cervical squamous cell carcinoma and endocervical adenocarcinoma (CESC), head and neck squamous cell carcinoma (HNSC), lung squamous cell carcinoma (LUSC) or pancreatic adenocarcinoma (PAAD).

41. The method of claim 39 wherein the one or more signatures are selected from SBS1, SBS2, SBS6, SBS10a, SBS10b, SBS13, SBS15, SBS20, SBS26, or SBS30, or any combination thereof.

42. The method of claim 39, 40 or 41 wherein the therapy is radiotherapy.

43. The method of claim 39, 40 or 41 wherein the therapy is chemotherapy.

44. The method of claim 39, 40 or 41 wherein the therapy is immunotherapy.

45. The method of claim 44 wherein the therapy is antibody therapy.

46. The method of any one of claims 39 to 40 wherein the presences of the one or more mutational signatures is detected using a probe, sequencing or nucleic acid amplification, or a combination thereof.
